(19) United States
(12) Patent Application Publication
(10) Pub. No.: US 2022/0387618 A1
(43) Pub. Date: Dec. 8, 2022

(54) METHODS OF TREATING HER2 POSITIVE BREAST CANCER WITH TUCATINIB IN COMBINATION WITH AN ANTI-HER2 ANTIBODY-DRUG CONJUGATE

(71) Applicant: Seagen Inc., Bothell, WA (US)

(72) Inventors: Luke Walker, Bothell, WA (US); Jorge Ramos, Bothell, WA (US); Aulma Parker, Bothell, WA (US); Anita Kulukian, Bothell, WA (US)

(21) Appl. No.: 17/775,492

(22) PCT Filed: Nov. 13, 2020
(86) PCT No.: PCT/US2020/060431

§ 371 Date: May 9, 2022

Related U.S. Application Data
(60) Provisional application No. 62/935,989, filed on Nov. 15, 2019.
(60) Provisional application No. 62/945,321, filed on Dec. 9, 2019.
(60) Provisional application No. 63/071,800, filed on Aug. 28, 2020.

(51) Int. Cl. A61K 47/68; A61K 31/517; A61K 45/06; A61P 35/00; A61P 35/04

(52) U.S. Cl. CPC A61K 47/6855; A61K 31/517; A61K 45/06; A61K 47/6803; A61P 35/00; A61P 35/04

(57) ABSTRACT

The present disclosure relates to a method for treating or ameliorating the effects of a HER2 positive breast cancer in a subject by administration of a combination of tucatinib and an anti-HER2 antibody-drug conjugate (e.g., ado-trastuzumab emtansine or trastuzumab deruxtecan). In some embodiments, the methods provided herein are useful for treating or ameliorating the effects of a brain metastasis in a subject having a HER2 positive breast cancer by administration of a combination of tucatinib and an anti-HER2 antibody-drug conjugate (e.g., ado-trastuzumab emtansine or trastuzumab deruxtecan).

### CROSS-REFERENCE TO RELATED APPLICATIONS

This application claims the benefit of U.S. Provisional Application No. 62/935,989, filed on Nov. 15, 2019, 62/945,321, filed on Dec. 9, 2019, and 63/071,800, filed on Aug. 28, 2020. The disclosure of the prior applications is hereby incorporated by reference in their entireties.

### BACKGROUND

Breast cancer is the most common form of cancer in women worldwide, and the second leading cause of cancer-related death in the United States. Approximately 20% of breast cancers overexpress the human epidermal growth factor receptor 2 (HER2). HER2 is a transmembrane tyrosine kinase receptor that mediates cell growth, differentiation, and survival. Tumors that overexpress HER2 are more aggressive and historically have been associated with poorer overall survival (OS) compared to HER2 negative cancers. Cancers that are characterized by the overexpression of HER2 (referred to as HER2 positive cancers) are often correlated with poor prognosis and/or are resistant to many standard therapies.

The introduction of HER2-targeted therapy using either antibody-based therapy or a small molecule tyrosine kinase inhibitor (TKI) has led to improvements in disease-free survival (DFS), progression-free survival (PFS), and OS in both the adjuvant and metastatic settings. Trastuzumab, a humanized anti-HER2 antibody, remains the backbone of treatment in the adjuvant and first-line metastatic settings, usually in combination with a taxane. Anti-HER2 therapy in combination with cytotoxic chemotherapy allows for concurrent treatment with agents having two different mechanisms of action, leading to greater efficacy than with either agent alone.

Despite the improvements in outcomes for early stage HER2+ breast cancer, up to a quarter of all patients treated with anti-HER2 therapy in the adjuvant setting relapse. The development of HER2 targeted therapies such as pertuzumab, trastuzumab deruxtecan (DS-8201a), and T-DM1 (ado-trastuzumab emtansine or trastuzumab emtansine) for metastatic HER2+ breast cancer has led to a meaningful prolongation in the median survival of these patients; however, essentially all patients in the metastatic setting ultimately progress. Treatment failures may result from primary or acquired resistance to HER2 blockade. There is evidence that dual targeting of HER2, either through combination of two different HER2-targeted antibodies or through use of an antibody-based therapy such as trastuzumab and a TKI, can lead to further improvements in efficacy in metastatic disease. In particular, combination of a small molecule TKI with an antibody-based therapy may be effective, as it may help overcome resistance to antibody-mediated inhibition through utilization of an alternative mechanism of receptor inhibition. Lapatinib, a dual epidermal growth factor receptor (EGFR)/HER2 oral TKI, has been shown to have increased activity in combination with trastuzumab compared to lapatinib alone, even when given to patients who have previously progressed on prior trastuzumab-based therapy. Use of lapatinib, however, has been limited by the anti-EGFR/human epidermal growth factor receptor 1 (HER1) activity of the drug, which results in toxicities such as rash, diarrhea, and fatigue.

The current standard of care for patients with HER2+ metastatic disease consists of treatment with pertuzumab plus trastuzumab and a taxane as first-line treatment for metastatic disease, followed by T-DM1 in second line. Treatment options for patients who progress after treatment with both pertuzumab and T-DM1 remain relatively limited. Patients are generally treated with a continuation of anti-HER2 therapy (in the form of trastuzumab or lapatinib) in combination with cytotoxic chemotherapy, such as capecitabine. Combined HER2 therapy with trastuzumab and lapatinib can also be considered.

The treatment and prevention of brain metastases represents an unmet medical need in the post-trastuzumab era. Recent data suggest that the incidence of first relapse occurring in the brain is increasing in patients who have received trastuzumab-based adjuvant therapy, and approximately 30-50% of HER2+ patients with metastatic disease will develop brain metastases. The increasing prevalence of brain metastases in HER2+ breast cancer patients may be due to several factors. First, HER2+ breast cancer appears to display tropism for the brain. Second, with better control of non-CNS disease, patients may be living longer allowing brain metastases to become more of a critical clinical issue. Finally, the brain may represent a sanctuary site for HER2+ disease as large molecules, such as trastuzumab, do not penetrate the blood-brain barrier. Treatment options for brain metastases are limited. There is no specific systemic treatment regimen approved for brain metastases, and treatment currently relies heavily on the use of local therapies such as whole brain radiation therapy (WBRT), stereotactic radiation (SRS), or surgery. Patients may also receive chemotherapy alone, or capecitabine and either lapatinib or trastuzumab, although brain response rates are generally modest. The development of HER2-targeted systemic therapies with clinical benefit in both brain and non-CNS sites of disease could lead to improved clinical outcomes, both by improving overall PFS and OS as well as by avoiding or delaying the use of radiation therapy and its associated toxicities, including neurocognitive impairment.

Accordingly, there is a need for new therapies that are effective for the treatment of patients with HER2-positive breast cancer (e.g., patients with unresectable, locally advanced, or metastatic HER2-positive breast cancer, including patients with brain metastases).

All references cited herein, including patent applications, patent publications, and scientific literature, are herein incorporated by reference in their entirety, as if each individual reference were specifically and individually indicated to be incorporated by reference.

### SUMMARY

Provided herein are methods for treating or ameliorating a HER2 positive breast cancer in a subject in need thereof, the methods comprising administering to the subject a therapeutically effective amount of a combination therapy comprising tucatinib and an anti-HER2 antibody-drug conjugate.

Also provided herein are methods for treating of treating or ameliorating cancer in a subject in need thereof, the method comprising: (a) identifying the subject as having a HER2 positive breast cancer; and (b) administering to the subject a therapeutically effective amount of a combination therapy comprising tucatinib and an anti-HER2 antibody-drug conjugate.

Provided herein are methods for treating or ameliorating a HER2 positive breast cancer in a subject in need thereof, the methods comprising administering to the subject an effective amount of a combination therapy comprising tucatinib and an anti-HER2 antibody-drug conjugate, wherein following administration of the combination therapy, the subject exhibits progression-free survival of at least 7.5 months following administration of the combination therapy. For example, wherein the subject exhibits progression-free survival of at least eight months, at least nine months, or at least ten months following administration of the combination therapy.

In some embodiments, the methods comprise administering to the subject an effective amount of a combination therapy comprising tucatinib and ado-trastuzumab emtansine, wherein following administration of the combination therapy, the subject exhibits progression-free survival of at least 7.5 months following administration of the combination therapy. For example, wherein the subject exhibits progression-free survival of at least eight months, at least nine months, or at least ten months following administration of the combination therapy.

Also provided herein are methods for treating or ameliorating a HER2 positive breast cancer in a subject in need thereof, methods comprising administering to the subject an effective amount of a combination therapy comprising tucatinib and an anti-HER2 antibody-drug conjugate, wherein following administration of the combination therapy, the subject exhibits an overall survival of at least eighteen months following administration of the combination therapy. For example, wherein the subject exhibits an overall survival of at least nineteen months, at least twenty-two months, at least twenty-six months, or at least thirty months following administration of the combination therapy.

In some embodiments, the methods comprise administering to the subject an effective amount of a combination therapy comprising tucatinib and ado-trastuzumab emtansine, wherein following administration of the combination therapy, the subject exhibits an overall survival of at least eighteen months following administration of the combination therapy. For example, wherein the subject exhibits an overall survival of at least nineteen months, at least twenty-two months, at least twenty-six months, or at least thirty months following administration of the combination therapy.

In some of the embodiments as provided herein, the subject has a brain metastasis.

Accordingly, provided herein are methods of treating or ameliorating brain metastasis in a subject having HER2 positive breast cancer, the methods comprising administering to the subject an effective amount of a combination therapy comprising tucatinib and an anti-HER2 antibody-drug conjugate. In some embodiments, the time to additional intervention (e.g., radiation, surgery, or a combination thereof) for treatment of the brain metastasis in the subject has been increased. In some embodiments, the need for additional intervention (e.g., radiation, surgery, or a combination thereof) for treatment of the brain metastasis in the subject has been prevented. In some embodiments, regression of an existing brain metastasis in the subject has been promoted. In some embodiments, the size of an existing brain metastasis in the subject has been reduced.

In some embodiments, the methods comprise administering to the subject an effective amount of a combination therapy comprising tucatinib and ado-trastuzumab emtansine. In some embodiments, the time to additional intervention (e.g., radiation, surgery, or a combination thereof) for treatment of the brain metastasis in the subject has been increased. In some embodiments, the need for additional intervention (e.g., radiation, surgery, or a combination thereof) for treatment of the brain metastasis in the subject has been prevented. In some embodiments, regression of an existing brain metastasis in the subject has been promoted. In some embodiments, the size of an existing brain metastasis in the subject has been reduced.

This disclosure also provides methods for treating or ameliorating a HER2 positive breast cancer in a subject in need thereof, wherein the subject has brain metastasis, the methods comprising administering to the subject an effective amount of a combination therapy comprising tucatinib and an anti-HER2 antibody-drug conjugate, wherein following administration of the combination therapy, the subject exhibits progression-free survival of at least 6 months following administration of the combination therapy. For example, the subject can exhibit progression-free survival of at least seven months or at least nine months following administration of the combination therapy.

In some embodiments, the methods comprise administering to the subject an effective amount of a combination therapy comprising tucatinib and ado-trastuzumab emtansine, wherein following administration of the combination therapy, the subject exhibits progression-free survival of at least 6 months following administration of the combination therapy. For example, the subject can exhibit progression-free survival of at least seven months or at least nine months following administration of the combination therapy.

Methods for treating or ameliorating a HER2 positive breast cancer in a subject in need thereof are also provided, the methods comprising administering to the subject an effective amount of a combination therapy comprising tucatinib and an anti-HER2 antibody-drug conjugate, wherein the subject exhibits a greater than 40% reduction in the risk of disease progression or death as compared to a subject administered the anti-HER2 antibody-drug conjugate alone. For example, the subject can exhibit a greater than 45% reduction in the risk of disease progression or death as compared to a subject administered the anti-HER2 antibody-drug conjugate alone.

In some embodiments, the methods comprise administering to the subject an effective amount of a combination therapy comprising tucatinib and ado-trastuzumab emtansine, wherein the subject exhibits a greater than 40% reduction in the risk of disease progression or death as compared to a subject administered ado-trastuzumab emtansine alone. For example, the subject can exhibit a greater than 45% reduction in the risk of disease progression or death as compared to a subject administered ado-trastuzumab emtansine alone.

Provided herein are methods for treating or ameliorating a HER2 positive breast cancer in a subject in need thereof, the methods comprising administering to the subject an effective amount of a combination therapy comprising tucatinib and an anti-HER2 antibody-drug conjugate, wherein the subject exhibits a greater than 30% reduction in the risk of death as compared to a subject administered the anti-HER2 antibody-drug conjugate alone.

In some embodiments, the methods comprise administering to the subject an effective amount of a combination therapy comprising tucatinib and ado-trastuzumab emtansine, wherein the subject exhibits a greater than 30% reduction in the risk of death as compared to a subject administered ado-trastuzumab emtansine alone.

Also provided herein are methods for treating or ameliorating a HER2 positive breast cancer in a subject in need thereof, wherein the subject has a brain metastasis, the methods comprising administering to the subject an effective amount of a combination therapy comprising tucatinib and an anti-HER2 antibody-drug conjugate, wherein the subject exhibits a greater than 50% reduction in the risk of disease progression or death as compared to a subject administered the anti-HER2 antibody-drug conjugate alone.

In some embodiments, the methods comprise administering to the subject an effective amount of a combination therapy comprising tucatinib and ado-trastuzumab emtansine, wherein the subject exhibits a greater than 50% reduction in the risk of disease progression or death as compared to a subject administered ado-trastuzumab emtansine alone.

Further provided herein are methods for treating or ameliorating a HER2 positive breast cancer in a subject in need thereof, the methods comprising administering to the subject an effective amount of a combination therapy comprising tucatinib and an anti-HER2 antibody-drug conjugate, wherein following administration of the combination therapy for nine months, the subject has an estimated progression-free survival rate of greater than 40%. For example, wherein the subject has an estimated progression-free survival rate of greater than 45% following administration of the combination therapy for nine months.

In some embodiments, the methods comprise administering to the subject an effective amount of a combination therapy comprising tucatinib and ado-trastuzumab emtansine, wherein following administration of the combination therapy for nine months, the subject has an estimated progression-free survival rate of greater than 40%. For example, wherein the subject has an estimated progression-free survival rate of greater than 45% following administration of the combination therapy for nine months.

This disclosure also provides methods for treating or ameliorating a HER2 positive breast cancer in a subject in need thereof, the methods comprising administering to the subject an effective amount of a combination therapy comprising tucatinib and an anti-HER2 antibody-drug conjugate, wherein following administration of the combination therapy for twelve months, the subject has an estimated progression-free survival rate of greater than 25%. For example, wherein the subject has an estimated progression-free survival rate of greater than 30% following administration of the combination therapy for twelve months.

In some embodiments, the methods comprise administering to the subject an effective amount of a combination therapy comprising tucatinib and ado-trastuzumab emtansine, wherein following administration of the combination therapy for twelve months, the subject has an estimated progression-free survival rate of greater than 25%. For example, wherein the subject has an estimated progression-free survival rate of greater than 30% following administration of the combination therapy for twelve months.

Methods for treating or ameliorating a HER2 positive breast cancer in a subject in need thereof are provided herein, the methods comprising administering to the subject an effective amount of a combination therapy comprising tucatinib and an anti-HER2 antibody-drug conjugate, wherein following administration of the combination therapy for fifteen months, the subject has an estimated progression-free survival rate of greater than 20%. For example, wherein the subject has an estimated progression-free survival rate of greater than 25% following administration of the combination therapy for fifteen months.

In some embodiments, the methods comprise administering to the subject an effective amount of a combination therapy comprising tucatinib and ado-trastuzumab emtansine, wherein following administration of the combination therapy for fifteen months, the subject has an estimated progression-free survival rate of greater than 20%. For example, wherein the subject has an estimated progression-free survival rate of greater than 25% following administration of the combination therapy for fifteen months.

Provided herein are methods for treating or ameliorating a HER2 positive breast cancer in a subject in need thereof, the methods comprising administering to the subject an effective amount of a combination therapy comprising tucatinib and an anti-HER2 antibody-drug conjugate, wherein following administration of the combination therapy for twenty-four months, the subject has an estimated overall survival rate of greater than 35%. For example, wherein the subject has an estimated overall survival rate of greater than 40% following administration of the combination therapy for twenty-four months.

In some embodiments, the methods comprise administering to the subject an effective amount of a combination therapy comprising tucatinib and ado-trastuzumab emtansine, wherein following administration of the combination therapy for twenty-four months, the subject has an estimated overall survival rate of greater than 35%. For example, wherein the subject has an estimated overall survival rate of greater than 40% following administration of the combination therapy for twenty-four months.

Also provided herein are methods for treating or ameliorating a HER2 positive breast cancer in a subject in need thereof, the methods comprising administering to the subject an effective amount of a combination therapy comprising tucatinib and an anti-HER2 antibody-drug conjugate, wherein following administration of the combination therapy for thirty months, the subject has an estimated overall survival rate of greater than 30%. For example, wherein the subject has an estimated overall survival rate of greater than 40% following administration of the combination therapy for thirty months.

In some embodiments, the methods comprise administering to the subject an effective amount of a combination therapy comprising tucatinib and ado-trastuzumab emtansine, wherein following administration of the combination therapy for thirty months, the subject has an estimated overall survival rate of greater than 30%. For example, wherein the subject has an estimated overall survival rate of greater than 40% following administration of the combination therapy for thirty months.

Further provided herein are methods for treating or ameliorating a HER2 positive breast cancer in a subject in need thereof, wherein the subject has a brain metastasis, the methods comprising administering to the subject an effective amount of a combination therapy comprising tucatinib and an anti-HER2 antibody-drug conjugate, wherein following administration of the combination therapy for nine months, the subject has an estimated progression-free survival rate of greater than 30%. For example, wherein the subject has an estimated progression-free survival rate of greater than 40% following administration of the combination therapy for nine months.

In some embodiments, the methods comprise administering to the subject an effective amount of a combination therapy comprising tucatinib and ado-trastuzumab emtansine, wherein following administration of the combination therapy for nine months, the subject has an estimated progression-free survival rate of greater than 30%. For example, wherein the subject has an estimated progression-free survival rate of greater than 40% following administration of the combination therapy for nine months.

This disclosure also provides methods for treating or ameliorating a HER2 positive breast cancer in a subject in need thereof, wherein the subject has a brain metastasis, the methods comprising administering to the subject an effective amount of a combination therapy comprising tucatinib and an anti-HER2 antibody-drug conjugate, wherein following administration of the combination therapy for twelve months, the subject has an estimated progression-free survival rate of greater than 15%. For example, wherein following administration of the combination therapy for twelve months, the subject has an estimated progression-free survival rate of greater than 20%.

In some embodiments, the methods comprise administering to the subject an effective amount of a combination therapy comprising tucatinib and ado-trastuzumab emtansine, wherein following administration of the combination therapy for twelve months, the subject has an estimated progression-free survival rate of greater than 15%. For example, wherein following administration of the combination therapy for twelve months, the subject has an estimated progression-free survival rate of greater than 20%.

Methods for treating or ameliorating a HER2 positive breast cancer in a subject in need thereof are provided herein, the methods comprising administering to the subject an effective amount of a combination therapy comprising tucatinib and an anti-HER2 antibody-drug conjugate and an effective amount of an anti-diarrheal agent. Also provided herein are method of reducing the severity or incidents of diarrhea, or preventing diarrhea in a subject having a HER2 positive breast cancer and being treated with an effective amount of a combination therapy comprising tucatinib and an anti-HER2 antibody-drug conjugate, the method comprising administering an effective amount of an anti-diarrheal agent prophylactically. This disclosure also provides methods of reducing the likelihood of a subject developing diarrhea, wherein the subject has a HER2 positive breast cancer and is being treated with an effective amount of a combination therapy comprising tucatinib and an anti-HER2 antibody-drug conjugate, the method comprising administering an effective amount of an anti-diarrheal agent prophylactically. In some such methods, the anti-HER2 antibody drug conjugate is ado-trastuzumab emtansine. In some such methods, the combination therapy and the anti-diarrheal agent can be administered concurrently. In some such methods, the anti-diarrheal agent is administered prior to administration of the combination therapy. In some embodiments, the subject is exhibiting symptoms of diarrhea. In other embodiments, the subject is not exhibiting symptoms of diarrhea.

In some embodiments, the tucatinib is administered twice daily. In some embodiments, the HER2 positive breast cancer is unresectable or metastatic. In some embodiments, the subject was previously treated with two or more anti-HER2-based regimens. In some embodiments, the subject has not been previously treated with an anti-HER2 and/or an anti-EGFR tyrosine kinase inhibitor. In some embodiments, the wherein the anti-HER2/EGFR tyrosine kinase inhibitor is selected from the group consisting of tucatinib, lapatinib, neratinib, or afatinib. In some embodiments, the subject has not been previously treated with an anti-HER2 antibody-drug conjugate. In some embodiments, the anti-HER2 antibody-drug conjugate is selected from the group consisting of ado-trastuzumab, or trastuzumab deruxtecan. In some embodiments, the subject has not been previously treated with an anthracycline. In some embodiments, the anthracycline is selected from the group consisting of doxorubicin, epirubicin, mitoxantrone, idarubicin, liposomal doxorubicin, and combinations thereof.

Also provided herein are methods for treating a HER2 positive breast cancer in a subject that has exhibited an adverse event after starting treatment with a combination therapy comprising tucatinib and anti-HER2 antibody-drug conjugate at an initial dosage level, comprising administering to the subject at least one component of the combination therapy at a reduced dosage level.

In some embodiments, the tucatinib is administered to the subject at an initial dose of about 150 mg to about 650 mg. In some embodiments, the tucatinib is administered to the subject at an initial dose of about 300 mg. In some embodiments, the tucatinib is administered to the subject at a reduced dose of about 125 mg to about 275 mg. In some embodiments, the tucatinib is administered to the subject at a reduced dose of about 250 mg. In some embodiments, the tucatinib is administered to the subject at a reduced dose of about 200 mg. In some embodiments, the tucatinib is administered to the subject at a reduced dose of about 150. In some embodiments, the anti-HER2 antibody-drug conjugate is administered to the subject at an initial dose of about 3 mg/kg to about 7 mg/kg.

In some embodiments, the anti-HER2 antibody-drug conjugate is trastuzumab deruxtecan. In some embodiments, the anti-HER2 antibody-drug conjugate is ado-trastuzumab emtansine.

In some embodiments, the trastuzumab deruxtecan is administered to the subject at an initial dose of about 5.4 mg/kg. In some embodiments, the trastuzumab deruxtecan is administered to the subject at a reduced dose of about 4.4 mg/kg. In some embodiments, the trastuzumab deruxtecan is administered to the subject at a reduced dose of about 3.2 mg/kg.

In some embodiments, the ado-trastuzumab emtansine is administered to the subject at an initial dose of about 3.6 mg/kg. In some embodiments, the ado-trastuzumab emtansine is administered to the subject at a reduced dose of about 3 mg/kg. In some embodiments, the ado-trastuzumab emtansine is administered to the subject at a reduced dose of about 2.4 mg/kg.

In some embodiments, of any of the methods described herein, the ado-trastuzumab emtansine is administered to the subject at a reduced dose of about 2.4 mg/kg.

In some embodiments, of any of the methods described herein, the anti-HER2 antibody-drug conjugate is ado-trastuzumab emtansine.

In some embodiments, of any of the methods described herein, the anti-HER2 antibody-drug conjugate is trastuzumab deruxtecan.

In some embodiments, of any of the methods described herein, the trastuzumab deruxtecan is administered to the subject at a dose of about 3 mg/kg to about 7 mg/kg.

In some embodiments, of any of the methods described herein, the trastuzumab deruxtecan is administered to the subject at a dose of about 5.4 mg/kg.

In some embodiments, of any of the methods described herein, the trastuzumab deruxtecan is administered to the subject once per 21 day treatment cycle.

In some embodiments, of any of the methods described herein, the ado-trastuzumab emtansine is administered to the subject at a dose of about 3 mg/kg to about 7 mg/kg.

In some embodiments, of any of the methods described herein, the ado-trastuzumab emtansine is administered to the subject at a dose of about 3.6 mg/kg.

In some embodiments, of any of the methods described herein, the ado-trastuzumab emtansine is administered to the subject once per 21 day treatment cycle.

In some embodiments, of any of the methods described herein, wherein the administration of the tucatinib, or a salt or solvate thereof, increases the overall amount of HER2 in a solid tumor.

In some embodiments, of any of the methods described herein, wherein the overall amount of HER2 in the solid tumor is determined by western blot analysis.

In some embodiments, of any of the methods described herein, wherein the administration of the tucatinib, or a salt or solvate thereof, increases the amount of membrane-bound HER2 in the solid tumor.

In some embodiments, of any of the methods described herein, wherein the amount of membrane-bound HER2 in the solid tumor is determined by quantitative fluorescence activated cell sorting (qFACS).

In some embodiments, of any of the methods described herein, the administration of the tucatinib, or a salt or solvate thereof, increases a dwell time of HER2 at the cell surface.

In some embodiments, of any of the methods described herein, the administration of the tucatinib, or a salt or solvate thereof, increases an internalization of membrane-bound HER2.

In some embodiments, of any of the methods described herein, the administration of the tucatinib, or a salt or solvate thereof, increases a lysosomal degradation of HER2.

### DESCRIPTION OF THE DRAWINGS

FIG. 1 shows isobologram analysis of a 96-hour cytotoxicity assay.

FIG. 2A shows the results of various treatments and combinations thereof in a BT-474 cell line-derived xenograft model.

FIG. 2B shows the results of various treatments and combinations thereof in two HER2+ patient-derived (PDX) breast cancer models.

FIG. 3 shows a summary of partial responses (PR) and complete responses (CR) in various cancer models following treatment with tucatinib, T-DMI1, and a combination of tucatinib and T-DM1.

FIG. 4 illustrates a study schema for the clinical trial described in Example 3.

FIG. 5 illustrates a pharmacokinetics (PK) sub-study as described in Example 3.

FIG. 6 provides the amino acid sequence of the heavy (SEQ. ID NO. 1) and light chains (SEQ. ID NO. 2) of trastuzumab and the light chain variable domain (SEQ. ID NO. 3) and the heavy chain variable domain (SEQ. ID NO. 4) of trastuzumab.

FIG. 7 shows a schematic of a proposed mechanism of action for tucatinib.

FIG. 8 shows changes to total HER2 protein levels and HER2 membrane-bound protein levels upon treatment with tucatinib in various cancer cell lines.

FIGS. 9A and 9B shows schematics of internalization assays using Trastuzumab-AF488 and Trastuzumab-QF.

FIGS. 10A and 10B show dynamics of HER2 at the cell surface upon binding to antibody therapeutics.

FIGS. 11A, 11B, and 11C show a schematic of intracellular drug measurement studies, the structure of the primary T-DM1 catabolite, Lys-MCC-DM1, and the concentration of Lysine-MCC-DM1 measured over timepoints following administration of T-DM1 or a combination of T-DM1 and tucatinib.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure. For purposes of the present disclosure, the following terms are defined.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “or” as used herein should in general be construed non-exclusively. For example, a claim to “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

The group “A or B” is typically equivalent to the group “selected from the group consisting of A and B.”

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the disclosure described herein include “comprising,” “consisting,” and “consisting essentially of” aspects and embodiments. It is understood that aspects and variations of the embodiments described herein include “consisting of” and/or “consisting essentially of” aspects and variations. In some embodiments, methods consisting essentially of an administration step as disclosed herein include methods wherein a patient has failed a prior therapy (administered to the patient before the period of time) or has been refractory to such prior therapy, and/or wherein the cancer has metastasized or recurred. In some embodiments, methods consisting essentially of an administration step as disclosed herein include methods wherein a patient undergoes surgery, radiation, and/or other regimens prior to, substantially at the same time as, or following such an administration step as disclosed herein, and/or where the patient is administered other chemical and/or biological therapeutic agents following such an administration step as disclosed herein.

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95×, 0.96×, 0.97×, 0.98×, 0.99×, 1.01×, 1.02×, 1.03×, 1.04×, and 1.05×. Thus, “about X” is intended to teach and provide written description support for a claim limitation of, e.g., “0.98×.” The terms “about” and “approximately,” particularly in reference to a given quantity, encompass and describe the given quantity itself.

Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11 mg/kg” is equivalent to “about 7, about 9, or about 11 mg/kg.”

The term “comprising” as used herein should in general be construed as not excluding additional ingredients. For example, a claim to “a composition comprising A” would cover compositions that include A and B; A, B, and C; A, B, C, and D; A, B, C, D, and E; and the like. As used herein, the term “co-administering” includes sequential or simultaneous administration of tucatinib and an anti-HER2 antibody-drug conjugate (e.g., ado-trastuzumab emtansine). For example, the co-administered compounds are administered by the same route. In other instances, the co-administered compounds are administered via different routes. For example, one or two compounds can be administered orally, and the other compound(s) can be administered, e.g., sequentially or simultaneously, via intravenous, intramuscular, subcutaneous, or intraperitoneal injection. The simultaneously or sequentially administered compounds or compositions can be administered such that the anti-HER2 antibody-drug conjugate (e.g., ado-trastuzumab emtansine) and tucatinib are simultaneously present in a subject or in a cell at an effective concentration.

A “cancer” refers to a broad group of various diseases characterized by the uncontrolled growth of abnormal cells in the body. A “cancer” or “cancer tissue” can include a tumor.

In the context of cancer, the term “stage” refers to a classification of the extent of cancer. Factors that are considered when staging a cancer include but are not limited to tumor size, tumor invasion of nearby tissues, and whether the tumor has metastasized to other sites. The specific criteria and parameters for differentiating one stage from another can vary depending on the type of cancer. Cancer staging is used, for example, to assist in determining a prognosis or identifying the most appropriate treatment option(s).

One non-limiting example of a cancer staging system is referred to as the “TNM” system. In the TNM system, “T” refers to the size and extent of the main tumor, “N” refers to the number of nearby lymph nodes to which the cancer has spread, and “M” refers to whether the cancer has metastasized. “TX” denotes that the main tumor cannot be measured, “T0” denotes that the main tumor cannot be found, and “T1,” “T2,” “T3,” and “T4” denote the size or extent of the main tumor, wherein a larger number corresponds to a larger tumor or a tumor that has grown into nearby tissues. “NX” denotes that cancer in nearby lymph nodes cannot be measured, “NO” denotes that there is no cancer in nearby lymph nodes, and “N1,” “N2,” “N3,” and “N4” denote the number and location of lymph nodes to which the cancer has spread, wherein a larger number corresponds to a greater number of lymph nodes containing the cancer. “MX” denotes that metastasis cannot be measured, “M0” denotes that no metastasis has occurred, and “M1” denotes that the cancer has metastasized to other parts of the body.

As another non-limiting example of a cancer staging system, cancers are classified or graded as having one of five stages: “Stage 0,” “Stage I,” “Stage II,” “Stage III,” or “Stage IV.” Stage 0 denotes that abnormal cells are present, but have not spread to nearby tissue. This is also commonly called carcinoma in situ (CIS). CIS is not cancer, but may subsequently develop into cancer. Stages I, II, and III denote that cancer is present. Higher numbers correspond to larger tumor sizes or tumors that have spread to nearby tissues. Stage IV denotes that the cancer has metastasized. One of skill in the art will be familiar with the different cancer staging systems and readily be able to apply or interpret them.

The term “HER2” (also known as also known as HER2/neu, ERBB2, CD340, receptor tyrosine-protein kinase erbB-2, proto-oncogene Neu, and human epidermal growth factor receptor 2) refers to a member of the human epidermal growth factor receptor (HER/EGFR/ERBB) family of receptor tyrosine kinases. Amplification or overexpression of HER2 plays a significant role in the development and progression of certain aggressive types of cancer, including colorectal cancer, gastric cancer, lung cancer (e.g., non-small cell lung cancer (NSCLC)), biliary cancers (e.g., cholangiocarcinoma, gallbladder cancer), bladder cancer, esophageal cancer, melanoma, ovarian cancer, liver cancer, prostate cancer, pancreatic cancer, small intestine cancer, head and neck cancer, uterine cancer, cervical cancer, and breast cancer. Non-limiting examples of HER2 nucleotide sequences are set forth in GenBank reference numbers NP_001005862, NP_001289936, NP_001289937, NP_001289938, and NP_004448. Non-limiting examples of HER2 peptide sequences are set forth in GenBank reference numbers NP_001005862, NP_001276865, NP_001276866, NP_001276867, and NP_004439.

When HER2 is amplified or overexpressed in or on a cell, the cell is referred to as being “HER2 positive.” The level of HER2 amplification or overexpression in HER2 positive cells is commonly expressed as a score ranging from 0 to 3 (i.e., HER2 0, HER2 1+, HER2 2+, or HER2 3+), with higher scores corresponding to greater degrees of expression.

The term “HER2 positive-associated” with respect to a disease or disorder, as used herein refers to diseases or disorders associated with amplification or overexpression of HER2. Non-limiting examples of HER2 positive-associated diseases or disorders include, for example, HER2 positive breast cancer (e.g., “HER2 positive breast cancer-associated”).

The term “metastasis” is an art known term that refers to the spread of cancer cells from the place where they first formed (the primary site) to one or more other sites in a subject (one or more secondary sites). In metastasis, cancer cells break away from the original (primary) tumor, travel through the blood or lymph system, and form a new tumor (a metastatic tumor) in other organs or tissues of the body. The new, metastatic tumor includes the same or similar cancer cells as the primary tumor. At the secondary site, the tumor cell may proliferate and begin the growth or colonization of a secondary tumor at this distant site.

The term “metastatic cancer” (also known as “secondary cancer”) as used herein refers to a type of cancer that originates in one tissue type, but then spreads to one or more tissues outside of the (primary) cancer's origin. Following metastasis, the distal tumors can be said to be “derived from” the pre-metastasis tumor. For example, a “tumor derived from” a breast cancer refers to a tumor that is the result of a metastasized breast cancer. Metastatic brain cancer refers to cancer in the brain, i.e., cancer which originated in a tissue other than the brain and has metastasized to the brain.

The term “tucatinib,” also known as ONT-380 and ARRY-380, refers to the small molecule tyrosine kinase inhibitor that suppresses or blocks HER2 activation. Tucatinib has the following structure:

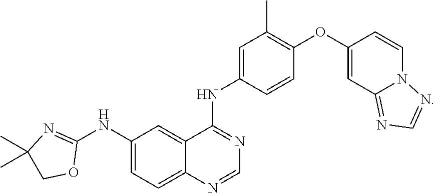 [SMILES: Cc1cc(Nc2ncnc3ccc(NC4=NC(C)(C)CO4)cc23)ccc1Oc1ccn2ncnc2c1]

In some instances, tucatinib can be in the form of a pharmaceutically acceptable salt.

The term “anti-HER2 antibody-drug conjugate” refers to an anti-HER2 antibody conjugated to a therapeutic agent (i.e., a drug) optionally via a linker.

An “anti-HER2 antibody”, as used herein, refers to an antibody that binds to the HER2 protein. Anti-HER2 antibodies used for the treatment of cancer are typically monoclonal, although polyclonal antibodies are not excluded by the term. Anti-HER2 antibodies inhibit HER2 activation or downstream signaling by various mechanisms. As non-limiting examples, anti-HER2 antibodies can prevent ligand binding, receptor activation or receptor signal propagation, result in reduced HER2 expression or localization to the cell surface, inhibit HER2 cleavage, or induce antibody-mediated cytotoxicity. Non-limiting examples of anti-HER2 antibodies that are suitable for use in the methods and compositions of the present invention include trastuzumab, pertuzumab, margetuximab, and combinations thereof.

The term “ado-trastuzumab emtansine”, also known as T-DM1, refers to an antibody-drug conjugate composed of trastuzumab, a thioether linker, and a derivative of the antimitotic agent maytansine (also known as DM1). Ado-trastuzumab emtansine is sold in the U.S. under the trade name KADCYCLA®. As used herein, “ado-trastuzumab emtansine” also includes biosimilars of trastuzumab, for example, Kanjinti (trastuzumab-anns).

The term “trastuzumab deruxtecan”, also known as DS-8201a, refers to an antibody-drug conjugate composed of trastuzumab, a linker, and a topoisomerase I inhibitor deruxtecan. Trastuzumab deruxtecan is sold in the U.S. under the trade name ENHIERTU®. As used herein, “trastuzumab deruxtecan” also includes biosimilars of trastuzumab, for example, Kanjinti (trastuzumab-anns).

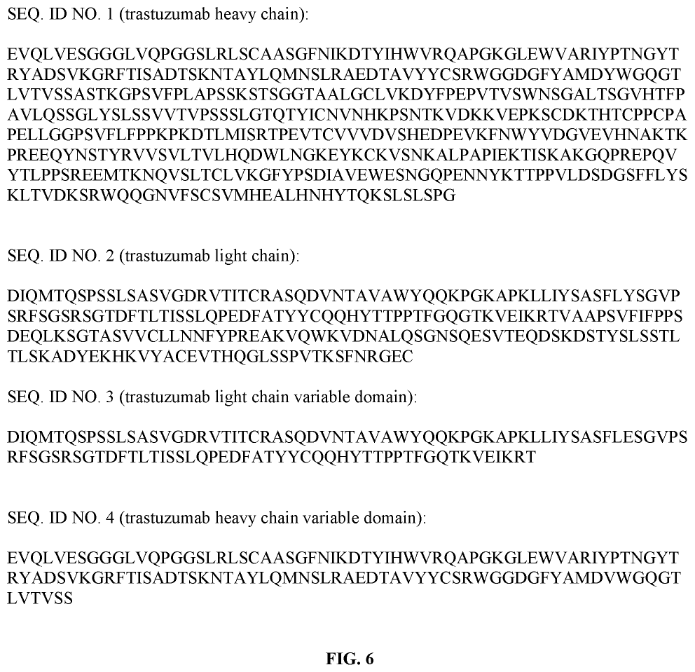

A “biosimilar” as used herein refers to an antibody or antigen-binding fragment that has the same primary amino acid sequence as compared to a reference antibody (e.g., trastuzumab) and optionally, may have detectable differences in post-translation modifications (e.g., glycosylation and/or phosphorylation) as compared to the reference antibody (e.g., a different glycoform). As a reference, the amino acid sequence of the heavy chain of trastuzumab is provided as SEQ. ID NO. 1, the light chain of trastuzumab is provided as SEQ. ID NO. 2, the light chain variable domain (SEQ. ID NO. 3), and the heavy chain variable domain (SEQ. ID NO. 4) (see also FIG. 6 and U.S. Pat. No. 5,821,337, which is incorporated herein in its entirety).

In some embodiments, a biosimilar is an antibody or antigen-binding fragment thereof that has a light chain that has the same primary amino acid sequence as compared to a reference antibody (e.g., trastuzumab) and a heavy chain that has the same primary amino acid sequence as compared to the reference antibody. In some examples, a biosimilar is an antibody or antigen-binding fragment thereof that has a light chain that includes the same light chain variable domain sequence as a reference antibody (e.g., trastuzumab) and a heavy chain that includes the same heavy chain variable domain sequence as a reference antibody. In some embodiments, a biosimilar can have a similar glycosylation pattern as compared to the reference antibody (e.g., trastuzumab). In other embodiments, a biosimilar can have a different glycosylation pattern as compared to the reference antibody (e.g., trastuzumab).

The term “tumor growth inhibition (TGI) index” refers to a value used to represent the degree to which an agent (e.g., tucatinib, an anti-HER2 antibody-drug conjugate such as ado-trastuzumab emtansine, trastuzumab deruxtecan, or a combination thereof) inhibits the growth of a tumor when compared to an untreated control. The TGI index is calculated for a particular time point (e.g., a specific number of days into an experiment or clinical trial) according to the following formula:

$$TGI = 1 - ( \frac{V o l u m {e}_{t r e a t e d ( Tx Day X )} - V o l u m {e}_{t r e a t e d ( Tx Day 0 )}}{V o l u m {e}_{c o n t r o l ( Tx Day X )} - V o l u m {e}_{c o n t r o l ( Tx Day 0 )}} ) \times 100 \% ,$$

where “Tx Day 0” denotes the first day that treatment is administered (i.e., the first day that an experimental therapy or a control therapy (e.g., vehicle only) is administered) and “Tx Day X” denotes X number of days after Day 0. Typically, mean volumes for treated and control groups are used. As a non-limiting example, in an experiment where study day 0 corresponds to “Tx Day 0” and the TGI index is calculated on study day 28 (i.e., “Tx Day 28”), if the mean tumor volume in both groups on study day 0 is 250 mm³ and the mean tumor volumes in the experimental and control groups are 125 mm³ and 750 mm³, respectively, then the TGI index on day 28 is 125%.

As used herein, the term “synergistic” or “synergy” refers to a result that is observed when administering a combination of components or agents (e.g., a combination of tucatinib and an anti-HER2 antibody-drug conjugate such as ado-trastuzumab emtansine or trastuzumab deruxtecan) produces an effect (e.g., inhibition of tumor growth, prolongation of survival time) that is greater than the effect that would be expected based on the additive properties or effects of the individual components. In some embodiments, synergism is determined by performing a Bliss analysis (see, e.g., Foucquier et al. Pharmacol. Res. Perspect. (2015) 3(3):e00149; hereby incorporated by reference in its entirety for all purposes). The Bliss Independence model assumes that drug effects are outcomes of probabilistic processes, and assumes that the drugs act completely independently (i.e., the drugs do not interfere with one another (e.g., the drugs have different sites of action) but each contributes to a common result).

The observed effect of a combination of drugs can be based on, for example, the TGI index, tumor size (e.g., volume, mass), an absolute change in tumor size (e.g., volume, mass) between two or more time points (e.g., between the first day a treatment is adminstered and a particular number of days after treatment is first administered), the rate of change of tumor size (e.g., volume, mass) between two or more time points (e.g., between the first day a treatment is adminstered and a particular number of days after treatment is first administered), or the survival time of a subject or a population of subjects. When the TGI index is taken as a measure of the observed effect of a combination of drugs, the TGI index can be determined at one or more time points. When the TGI index is determined at two or more time points, in some instances the mean or median value of the multiple TGI indices can be used as a measure of the observed effect. Furthermore, the TGI index can be determined in a single subject or a population of subjects. When the TGI index is determined in a population, the mean or median TGI index in the population (e.g., at one or more time points) can be used as a measure of the observed effect. When tumor size or the rate of tumor growth is used as a measure of the observed effect, the tumor size or rate of tumor growth can be measured in a subject or a population of subjects. In some instances, the mean or median tumor size or rate of tumor growth is determined for a subject at two or more time points, or among a population of subjects at one or more time points. When survival time is measured in a population, the mean or median survival time can be used as a measure of the observed effect.

When TGI indices are taken as a measure of the observed effects, the TGI indices can be determined at one or more time points. When TGI indices are determined at two or more time points, in some instances the mean or median values can be used as measures of the observed effects. Furthermore, the TGI indices can be determined in a single subject or a population of subjects in each treatment group. When the TGI indices are determined in populations of subjects, the mean or median TGI indices in each population (e.g., at one or more time points) can be used as measures of the observed effects. When tumor sizes or the rates of tumor growth are used as measures of the observed effects, the tumor sizes or rates of tumor growth can be measured in a subject or a population of subjects in each treatment group. In some instances, the mean or median tumor sizes or rates of tumor growth are determined for subjects at two or more time points, or among populations of subjects at one or more time points. When survival time is measured in a population, mean or median survival times can be used as measures of the observed effects.

In some embodiments, a combination of tucatinib and an anti-HER2 antibody-drug conjugate (e.g., ado-trastuzumab emtansine, or trastuzumab deruxtecan) is considered to be synergistic when the combination produces an observed TGI index that is greater than the predicted TGI index for the combination of drugs (e.g., when the predicted TGI index is based upon the assumption that the drugs produced a combined effect that is additive). In some instances, the combination is considered to be synergistic when the observed TGI index is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the predicted TGI index for the combination of drugs.

In some embodiments, the rate of tumor growth (e.g., the rate of change of the size (e.g., volume, mass) of the tumor) is used to determine whether a combination of drugs is synergistic (e.g., the combination of drugs is synergistic when the rate of tumor growth is slower than would be expected if the combination of drugs produced an additive effect). In other embodiments, survival time is used to determine whether a combination of drugs is synergistic (e.g., a combination of drugs is synergistic when the survival time of a subject or population of subjects is longer than would be expected if the combination of drugs produced an additive effect).

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer. As used herein, the terms “treatment” and “treating” when referring, e.g., to the treatment of a cancer, are not intended to be absolute terms. For example, “treatment of cancer” and “treating cancer”, as used in a clinical setting, is intended to include obtaining beneficial or desired clinical results and can include an improvement in the condition of a subject having cancer. Beneficial or desired clinical results include, but are not limited to, one or more of the following: reducing the proliferation of (or destroying) neoplastic or cancerous cells, inhibiting metastasis of neoplastic cells, a decrease in metastasis in a subject, shrinking or decreasing the size of a tumor, change in the growth rate of one or more tumor(s) in a subject, an increase in the period of remission for a subject (e.g., as compared to the one or more metric(s) in a subject having a similar cancer receiving no treatment or a different treatment, or as compared to the one or more metric(s) in the same subject prior to treatment), decreasing symptoms resulting from a disease, increasing the quality of life of those suffering from a disease (e.g., assessed using FACT-G or EORTC-QLQC30), decreasing the dose of other medications required to treat a disease, delaying the progression of a disease, and/or prolonging survival of subjects having a disease.

The term “prophylactic” or “prophylactically” refers to any type of intervention or process performed on, or the administration of an active agent to, the subject with the objective of protecting or preventing a disease or condition from developing or at least not developing fully (e.g., to reduce the symptoms or severity of the disease or condition) such as in the development of a side effect (e.g., diarrhea).

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects).

In other embodiments of the disclosure, tumor regression (e.g., brain metastasis regression) can be observed and continue for a period of at least about 20 days, at least about 30 days, at least about 40 days, at least about 50 days, or at least about 60 days.

As used herein, “subtherapeutic dose” means a dose of a therapeutic compound (e.g., tucatinib) that is lower than the usual or typical dose of the therapeutic compound when administered alone for the treatment of a hyperproliferative disease (e.g., cancer).

“Simultaneous administration,” as used herein, means that the two or more therapies (e.g., in a combination therapy) are administered with a time separation of no more than about 15 minutes, such as no more than about any of 10, 5, or 1 minutes. When the two or more therapies are administered simultaneously, the two or more therapies can be contained in the same composition (e.g., a composition comprising both a first and second therapy) or in separate compositions (e.g., a first therapy in one composition and a second therapy is contained in another composition).

As used herein, the term “sequential administration” means that the two or more therapies (e.g., in a combination therapy) are administered with a time separation of more than about 15 minutes, such as more than about any of 20, 30, 40, 50, 60, or more minutes. Any of the two or more therapies may be administered first. The two or more therapies are contained in separate compositions, which may be contained in the same or different packages or kits.

As used herein, the term “concurrent administration” means that the administration of two or more therapies (e.g., in a combination therapy) overlap with each other. For example, the two or more therapies may be administered in the same day, or with a time separation of within one day, within two days, within three days, within four days, within five days, within six days, within seven days, within ten days, within fourteen days, or within twenty-one days.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., breast cancer) does not get worse. Progression-free survival may include the amount of time patients have experienced a complete response or a partial response, as well as the amount of time patients have experienced stable disease.

As used herein, “overall response rate” or “ORR” refers to the sum of complete response (CR) rate and partial response (PR) rate.

As used herein, “overall survival” or “OS” refers to the percentage of individuals in a group who are likely to be alive after a particular duration of time.

The term “weight-based dose”, as referred to herein, means that a dose administered to a subject is calculated based on the weight of the subject. For example, when a subject with 60 kg body weight requires 3.6 mg/kg of an agent, such as ado-trastuzumab emtansine or trastuzumab deruxtecan, one can calculate and use the appropriate amount of the agent (i.e., 216 mg) for administration to said subject.

The use of the term “fixed dose” with regard to a method of the disclosure means that two or more different agents (e.g., tucatinib and an anti-HER2 antibody-drug conjugate such as ado-trastuzumab emtansine or trastuzumab deruxtecan) are administered to a subject in particular (fixed) ratios with each other. In some embodiments, the fixed dose is based on the amount (e.g., mg) of the agents. In certain embodiments, the fixed dose is based on the concentration (e.g., mg/ml) of the agents. For example, a 1:1.5 ratio of tucatinib to an anti-HER2 antibody-drug conjugate (e.g., ado-trastuzumab emtansine) administered to a subject can mean about 150 mg of tucatinib and about 225 mg of the anti-HER2 antibody-drug conjugate (e.g., ado-trastuzumab emtansine) are administered to the subject.

The use of the term “flat dose” with regard to the methods and dosages of the disclosure means a dose that is administered to a subject without regard for the weight or body surface area (BSA) of the subject. The flat dose is therefore not provided as an mg/kg dose, but rather as an absolute amount of the agent (e.g., tucatinib and an anti-HER2 antibody-drug conjugate such as ado-trastuzumab emtansine or trastuzumab deruxtecan). For example, a subject with 60 kg body weight and a subject with 100 kg body weight would receive the same dose of tucatinib (e.g., 300 mg).

The phrase “pharmaceutically acceptable” indicates that the substance or composition must be compatible chemically and/or toxicologically, with the other ingredients comprising a formulation, and/or the mammal being treated therewith.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc. In some instances, the carrier is an agent that facilitates the delivery of a small molecule drug or antibody to a target cell or tissue. One of skill in the art will recognize that other pharmaceutical carriers are useful in the present disclosure.

The phrase “pharmaceutically acceptable salt” as used herein, refers to pharmaceutically acceptable organic or inorganic salts of a compound of the disclosure. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

As used herein, the term “solid dispersion” means a system in a solid state comprising at least two components, wherein one component is dispersed throughout the other component. For example, a solid dispersion as described herein can include one component of tucatinib dispersed throughout another component, such as a dispersion polymer.

As used herein, the term “amorphous” means a solid in a solid state that is a non-crystalline state. Amorphous solids generally possess crystal-like short range molecular arrangement, but no long range order of molecular packing as found in crystalline solids. The solid state form of a solid may be determined by polarized light microscopy, X-ray powder diffraction (“MUD”), differential scanning calorimetry (“DSC”), or other standard techniques known to those of skill in the art.

As used herein, the term “amorphous solid dispersion” means a solid comprising a drug substance and a dispersion polymer. The amorphous solid dispersion discussed herein comprises amorphous tucatinib and a dispersion polymer, wherein the amorphous solid dispersion contains tucatinib in a substantially amorphous solid state form. In certain embodiments, the substantially amorphous solid state form means that the tucatinib component in the amorphous solid dispersion is at least 80% amorphous tucatinib. In certain embodiments, the substantially amorphous solid state form means that the tucatinib component in the amorphous solid dispersion is at least 85% amorphous tucatinib. In certain embodiments, the substantially amorphous solid state form means that the tucatinib component in the amorphous solid dispersion is at least 90% tucatinib. In certain embodiments, the substantially amorphous solid state form means that the tucatinib component in the amorphous solid dispersion is at least 95% amorphous tucatinib.

As used herein, the term “dispersion polymer” means a polymer that allows for tucatinib to be dispersed throughout such that a solid dispersion may form. The dispersion polymer is preferably neutral or basic. The dispersion polymer may contain a mixture of two or more polymers. Examples of dispersion polymers include, but are not limited to, vinyl polymers and copolymers, vinylpyrrolidine vinylacetate copolymer (“PVP-VA”), polyvinyl alcohols, polyvinyl alcohol polyvinyl acetate copolymers, polyvinyl pyrrolidine (“PVP”), acrylate and methacrylate copolymers, methylacrylic acid methyl methacrylate copolymer (such as Eudragit®), polyethylene polyvinyl alcohol copolymers, polyoxyethylene-polyoxypropylene block copolymers (also referred to as poloxamers), graft copolymer comprised of polyethylene glycol, polyvinyl caprolactam and polyvinyl acetate (such as Soluplus®), cellulosic polymers, such as hydroxypropyl methyl cellulose acetate (“HPMCA”), hydroxypropyl methyl cellulose (“HPMC”), hydroxypropyl cellulose (“HPC”), methyl cellulose, hydroxyethyl methyl cellulose, hydroxyethyl cellulose, hydroxyethyl cellulose acetate, and hydroxyethyl ethyl cellulose, hydroxypropyl methyl cellulose acetate succinate (“HPMCAS”), hydroxypropyl methyl cellulose phthalate (“HPMCP”), carboxymethylethyl cellulose (“CMEC”), cellulose acetate phthalate (“CAP”), cellulose acetate succinate (“CAS”), hydroxypropyl methyl cellulose acetate phthalate (“HPMCAP”), cellulose acetate trimellitate (“CAT”), hydroxypropyl methyl cellulose acetate trimellitate (“HPMCAT”), and carboxymethylcellulose acetate butyrate (“CMCAB”), and the like.

As used herein, the term “spray drying” means processes involved in breaking up liquid mixtures into small droplets (atomization) and rapidly removing solvent from the mixture in a spray drying apparatus where there is a strong driving force for evaporation of solvent from the droplets. The phrase spray drying is used conventionally and broadly. Spray drying processes and spray drying equipment are described generally in Perry, Robert H., and Don W. Green (eds.). Perry's Chemical Engineers' Handbook. New York: McGraw-Hill, 2007 (8^{th} edition).

As used herein, “polymorphs” refer to distinct solids sharing the same molecular formula, yet each polymorph may have distinct solid state physical properties. A single compound may give rise to a variety of polymorphic forms where each form has different and distinct solid state physical properties, such as different solubility profiles, melting point temperatures, flowability, dissolution rates and/or different X-ray diffraction peaks. These practical physical characteristics are influenced by the conformation and orientation of molecules in the unit cell, which defines a particular polymorphic form of a substance. Polymorphic forms of a compound can be distinguished in a laboratory by X-ray diffraction spectroscopy, such as X-ray powder diffraction (“XRPD”), and by other methods, such as infrared spectrometry. Additionally, polymorphic forms of the same drug substance or active pharmaceutical ingredient can be administered by itself or formulated as a drug product (pharmaceutical composition) and are well known in the pharmaceutical art to affect, for example, the solubility, stability, flowability, tractability and compressibility of drug substances and the safety and efficacy of drug products. For more, see Hilfiker, Rolf (ed.), Polymorphism in the Pharmaceutical Industry. Weinheim, Germany: Wiley-VCH 2006.

“Administering” or “administration” refer to the physical introduction of a therapeutic agent to a subject, using any of the various methods and delivery systems known to those skilled in the art. Exemplary routes of administration include oral, intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

The terms “baseline” or “baseline value” used interchangeably herein can refer to a measurement or characterization of a symptom before the administration of the therapy or at the beginning of administration of the therapy. The baseline value can be compared to a reference value in order to determine the reduction or improvement of a symptom of a disease contemplated herein (e.g., breast cancer). The terms “reference” or “reference value” used interchangeably herein can refer to a measurement or characterization of a symptom after administration of the therapy. The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±two days, i.e., every twelve days to every sixteen days. “Once about every three weeks” can include every twenty-one days±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Description of the Embodiments

A. Methods for Treating Breast Cancer with Tucatinib in Combination with an Anti-HER2 Antibody-Drug Conjugate

The 2014 World Cancer Report from WHO (The World health organization) reports that breast cancer is the second most common cancer worldwide, accounting for just over 1 million new cases annually. It states that in 2000 about 400,000 women died from breast cancer, representing 1.6 percent of all female deaths. The proportion of breast cancer deaths was far higher in the rich countries (2 percent of all female deaths) than in economically poor regions (0.5 percent). Thus, breast cancer is strongly related to the Western lifestyle. As developing countries succeed in achieving lifestyles similar to Europe, North America, Australia, New Zealand and Japan, they will also encounter much higher cancer rates, particularly cancers of the breast. Recent data supports this prediction and show a 20% increase in breast cancer from 2008 to 2012. (Carter D. “New global survey shows an increasing cancer burden”. Am J Nurs. 2014 March; 114(3): 17).

In some aspects, the disclosure provides a method for treating cancer in a subject comprising administering a combination of tucatinib and an anti-HER2 antibody-drug conjugate as described herein. Also provided herein are methods for treating a cancer in a subject in need thereof, comprising administering to the subject a therapeutically effective amount of a combination therapy comprising tucatinib and an anti-HER2 antibody-drug conjugate. Further provided herein are methods for treating cancer in a subject in need thereof, the method comprising: (a) identifying the subject as having a cancer; and (b) administering to the subject a therapeutically effective amount of a combination therapy comprising tucatinib and an anti-HER2 antibody-drug conjugate.

In some embodiments, the antibody of the anti-HER2 antibody-drug conjugate is trastuzumab. In some embodiments, the antibody of the anti-HER2 antibody-drug conjugate is trastuzumab or a biosimilar thereof. In some embodiments, the anti-HER2 antibody-drug conjugate is selected from the group consisting of trastuzumab deruxtecan, ado-trastuzumab emtansine, and combinations thereof. In some embodiments, the anti-HER2 antibody-drug conjugate is trastuzumab deruxtecan. In some embodiments, the anti-HER2 antibody-drug conjugate is ado-trastuzumab emtansine.

In some embodiments, the cancer is a HER2 positive cancer. In some embodiments, the methods include treating a HER2 positive cancer in a subject in need thereof, the method comprising administering to the subject a therapeutically effective amount of a combination therapy comprising tucatinib and an anti-HER2 antibody-drug conjugate. In some embodiments, methods include treating a HER2 positive cancer in a subject in need thereof, the method comprising: (a) identifying the subject as having a HER2 positive cancer; and (b) administering to the subject a therapeutically effective amount of a combination therapy comprising tucatinib and an anti-HER2 antibody-drug conjugate.

In some embodiments, the methods include treating a HER2 positive cancer in a subject in need thereof, the method comprising administering to the subject a therapeutically effective amount of a combination therapy comprising tucatinib and trastuzumab deruxtecan. In some embodiments, methods include treating a HER2 positive cancer in a subject in need thereof, the method comprising: (a) identifying the subject as having a HER2 positive cancer; and (b) administering to the subject a therapeutically effective amount of a combination therapy comprising tucatinib and trastuzumab deruxtecan.

In some embodiments, the methods include treating a HER2 positive cancer in a subject in need thereof, the method comprising administering to the subject a therapeutically effective amount of a combination therapy comprising tucatinib and ado-trastuzumab emtansine. In some embodiments, methods include treating a HER2 positive cancer in a subject in need thereof, the method comprising: (a) identifying the subject as having a HER2 positive cancer; and (b) administering to the subject a therapeutically effective amount of a combination therapy comprising tucatinib and ado-trastuzumab emtansine.

In some embodiments, the HER2 positive cancer is selected from the group consisting of gastric adenocarcinoma, gastroesophageal junction (GEC) adenocarcinoma, esophageal adenocarcinoma, colorectal carcinoma (CRC), cholangiocarcinoma, gallbladder carcinoma, gastric cancer, lung cancer, biliary cancers, bladder cancer, esophageal cancer, melanoma, ovarian cancer, liver cancer, prostate cancer, pancreatic cancer, small intestine cancer, non-small cell lung cancer, head and neck cancer, uterine cancer, cervical cancer, brain cancer, and breast cancer. In some embodiments, the HER2 positive cancer is breast cancer.

In some aspects, the disclosure provides a method for treating breast cancer in a subject comprising administering a combination of tucatinib and an anti-HER2 antibody-drug conjugate (e.g., ado-trastuzumab emtansine or trastuzumab deruxtecan) as described herein. In some embodiments, the breast cancer is a HER2 positive breast cancer. In some embodiments, the cancer is determined to be HER2 positive using in situ hybridization, fluorescence in situ hybridization, or immunohistochemistry. In some embodiments, the breast cancer is metastatic. In some embodiments, the breast cancer has metastasized to the brain. In some embodiments, the breast cancer is locally advanced. In some embodiments, the breast cancer is unresectable.

In some embodiments, the disclosure provides a method for treating a HER2 positive cancer (e.g., breast cancer) in a subject that has exhibited an adverse event after starting treatment with a combination therapy comprising tucatinib and an anti-HER2 antibody-drug conjugate (e.g., ado-trastuzumab emtansine or trastuzumab deruxtecan) at an initial dosage level, comprising administering to the subject at least one component of the combination therapy at a reduced dosage level. In some embodiments, the disclosure provides a method for treating a HER2 positive breast cancer in a subject that has exhibited an adverse event after starting treatment with a combination therapy comprising tucatinib and trastuzumab deruxtecan at an initial dosage level, comprising administering to the subject at least one component of the combination therapy at a reduced dosage level. In some embodiments, the disclosure provides a method for treating a HER2 positive breast cancer in a subject that has exhibited an adverse event after starting treatment with a combination therapy comprising tucatinib and ado-trastuzumab emtansine at an initial dosage level, comprising administering to the subject at least one component of the combination therapy at a reduced dosage level.

In some embodiments, the tucatinib is administered to the subject at an initial dose of about 150 mg to about 650 mg. In some embodiments, the tucatinib is administered to the subject at an initial dose of about 300 mg. In some embodiments, the tucatinib is administered to the subject at a reduced dose of about 125 mg to about 275 mg. In some embodiments, the tucatinib is administered to the subject a reduced dose of about 250 mg, 200 mg, or 150 mg. In some embodiments, the tucatinib is administered to the subject a reduced dose of about 250 mg. In some embodiments, the tucatinib is administered to the subject a reduced dose of about 200 mg. In some embodiments, the tucatinib is administered to the subject a reduced dose of about 150 mg.

In some embodiments, the anti-HER2 antibody-drug conjugate (e.g., ado-trastuzumab emtansine or trastuzumab deruxtecan) is administered to the subject at an initial dose of about 3 mg/kg to about 7 mg/kg.

In some embodiments, the anti-HER2 antibody-drug conjugate is trastuzumab deruxtecan. In some embodiments, the trastuzumab deruxtecan is administered to the subject at an initial dose of about 5.4 mg/kg. In some embodiments, the trastuzumab deruxtecan is administered to the subject at a reduced dose of about 4.4 mg/kg. In some embodiments, the trastuzumab deruxtecan is administered to the subject at a reduced dose of about 3.2 mg/kg.

In some embodiments, the anti-HER2 antibody-drug conjugate is ado-trastuzumab emtansine. In some embodiments, the ado-trastuzumab emtansine is administered to the subject at an initial dose of 3.6 mg/kg. In some embodiments, the ado-trastuzumab emtansine is administered to the subject at a reduced dose of 3 mg/kg. In some embodiments, the ado-trastuzumab emtansine is administered to the subject at a reduced dose of 2.4 mg/kg.

In some embodiments, the subject has been previously treated with at least one therapeutic agent for the breast cancer. In some embodiments, the subject has been previously treated with at least two therapeutic agents for the breast cancer. In some embodiments, the subject has been previously treated with at least three, four, five, six, seven, eight, nine, ten, or more therapeutic agents for the breast cancer. In some embodiments, the subject has been previously treated at least one therapeutic agent for the breast cancer and did not respond to the treatment. In some embodiments, the subject has been previously treated with at least two therapeutic agents for the breast cancer and did not respond to the treatment. In some embodiments, the subject has been previously treated with at least three, four, five, six, seven, eight, nine, ten, or more therapeutic agents for the breast cancer and did not respond to the treatment. In some embodiments, the subject has been previously treated with at least one therapeutic agent for the breast cancer and relapsed after the treatment. In some embodiments, the subject has been previously treated with at least two therapeutic agents for the breast cancer and relapsed after the treatment. In some embodiments, the subject has been previously treated with at least three, four, five, six, seven, eight, nine, ten, or more therapeutic agents for the breast cancer and relapsed after the treatment. In some embodiments, the subject has been previously treated with at least one therapeutic agent for the breast cancer and experienced disease progression during the treatment. In some embodiments, the subject has been previously treated with at least two therapeutic agents for the breast cancer and experienced disease progression during the treatment. In some embodiments, the subject has been previously treated with at least three, four, five, six, seven, eight, nine, ten, or more therapeutic agents for the breast cancer and experienced disease progression during the treatment. In some embodiments, the at least one therapeutic agent is an anti-HER2-based regimen. In some embodiments, the at least two therapeutic agents are anti-HER2-based regimens. In some embodiments, the at least three, four, five, six, seven, eight, nine, ten, or more therapeutic agents are anti-HER2-based regimens. An “anti-HER2-based regimen” refers to an agent that exhibits HER2 inactivation activity (e.g., inhibiting or decreasing) (e.g., an anti-HER2-antibody or an anti-HER2 antibody drug conjugate) and that is administered to a subject alone or in combination with an anti-cancer agent.

In some embodiments, the at least one (e.g., at least two) therapeutic agent is an anti-HER2 antibody or an anti-HER2 antibody-drug conjugate. In some embodiments, the at least one (e.g., at least two) previously administered therapeutic agents are selected form the group consisting of trastuzumab, ado-trastuzumab emtansine, trastuzumab deruxtecan trastuzumab and a taxane, pertuzumab, ado-trastuzumab (T-DM1), and combinations thereof.

In some embodiments, at least one (e.g., at least two) therapeutic agent is an anti-HER2 antibody. In some embodiments, the at least one (e.g., at least two) therapeutic agent is an anti-HER2 antibody-drug conjugate.

In some embodiments, the subject has been previously treated with pertuzumab. In some embodiments, the subject has been previously treated with trastuzumab. In some embodiments, the subject has been previously treated with T-DM1. In some embodiments, the subject has been previously treated with trastuzumab, and pertuzumab. In some embodiments, the subject has been previously treated with trastuzumab, and T-DM1. In some embodiments, the subject has been previously treated with pertuzumab, and T-DM1. In some embodiments, the subject has been previously treated with trastuzumab, pertuzumab, and T-DM1. In some embodiments, the subject has been previously treated with trastuzumab, pertuzumab, and T-DM1. In some embodiments, the subject has been treated with trastuzumab and a taxane. In some embodiments, the subject has been treated with trastuzumab and a taxane and has also been treated with pertuzumab.

In some embodiments, the at least one (e.g., at least two) therapeutic agent is selected from the group consisting of chemotherapeutic agents such as doxorubicin and cyclophosphamide (e.g., ACTH regimen); a taxane (e.g., paclitaxel); docetaxel; docetaxel and carboplatin (e.g., TCH regimen); cisplatin; fluorouracil (5-FU); epirubicin; anthracyclines (e.g., doxorubicin); cyclophosphamide; vinorelbine; gemcitabine; kinase inhibitors, such as lapatinib; neratinib; pyrotinib; afatinib; poziotinib; abemaciclib; and pazopanib; hormone therapy, including, for example tamoxifen; toremifene; fulvestrant; aromatase inhibitors (e.g., anastrozole, exemestane, letrozole); and ovarian suppression (e.g., with goserelin or leuprolide); vaccines such as nelipepimut-S or E75 peptide combined with granulocyte macrophage-colony stimulating factor; and ETBX-021; combination therapies such as a chemotherapeutic agent and trastuzumab (and optionally pertuzumab); a taxane (e.g., paclitaxel) with trastuzumab; a taxane (e.g., paclitaxel) with trastuzumab and pertuzumab; cis-platin and fluoropyrimidine with trastuzumab; docetaxel and carboplatin with trastuzumab and pertuzumab; docetaxel and carboplatin with trastuzumab; docetaxel with trastuzumab and pertuzumab; docetaxel with trastuzumab; docetaxel and cyclophosphamide with trastuzumab; anthracycline and/or cyclophosphamide followed by paclitaxel with trastuzumab; pertuzumab with docetaxel; fluorouracil (5-FU), epirubicin, and cyclophosphamide with trastuzumab and/or pertuzumab; vinorelbine or gemcitabine with trastuzumab; anthracycline, a taxane and trastuzumab; doxorubicin with trastuzumab; lapatinib with capecitabine; lapatinib with trastuzumab; endocrine therapy with lapatinib and/or trastuzumab; pazopanib with lapatinib; anti-HER2 agents (e.g., trastuzumab) with CDK4/6 inhibitors (e.g., abemaciclib or palbociclib) such as abemaciclib with trastuzumab; palbociclib with trastuzumab, pertuzumab, and an aromatase inhibitor; palbociclib, trastuzumab (and optionally letrozole); palbociclib and T-DM1; palbociclib with trastuzumab, pertuzumab and anastrozole; ribociclib with trastuzumab or T-DM1; palbociclib with tucatinib and letrozole; anti-HER2 agents (e.g., trastuzumab, pertuzumab, T-DM1) with immunotherapy (e.g., with pembrolizumab, atezolizumab or nivolumab); anti-HER2 agents (e.g., trastuzumab, pertuzumab, T-DM1) with PI3K/AKT/mTOR inhibitors, for example, everolimus with trastuzumab and paclitaxel; everolimus with trastuzumab and vinorelbine; alpelisib with LJM716 and trastuzumab; alpelisib and T-DM1; taselisib with anti-HER2 agents (e.g., trastuzumab, trastuzumab emtansine, pertuzumab (and optionally paclitaxel)); and copanlisib with trastuzumab.

In some embodiments, the subject was previously treated with at least one anticancer therapy for the breast cancer. In some embodiments, the subject has been previously treated with one or more additional therapies for the breast cancer. For example, radiation (e.g., external beam radiation; brachytherapy), surgery (e.g., lumpectomy; mastectomy), and combinations thereof.

In some embodiments, the subject has a brain metastasis. In some embodiments, the subject is refractory to the previous treatment. In some embodiments, the subject developed one or more brain metastasis while on the previous treatment.

In some embodiments, the subject has not previously been treated with another therapeutic agent for the breast cancer. In some embodiments, the subject has not been previously treated with another therapeutic agent for the breast cancer within the past 1 day, 2 days, 3 days, 4 days, 5 days, 6 days, 7 days, 10 days, 2 weeks, 3 weeks, 4 weeks, 6 weeks, 2 months, 3 months, 7 months, 8 months, 9 months, 10 months, 11 months, 12 months, 15 months, 18 months, 2 years, 3 years, 4 years, 5 years, 6 years, 7 years, 8 years, 9 years or 10 years prior to being administered the therapeutically effective amount of tucatinib, or salt or solvate thereof. In some embodiments, the subject has not been previously treated with another therapeutic agent for the breast cancer within the past 12 months prior to being administered the therapeutically effective amount of tucatinib, or salt or solvate thereof. In some embodiments, the subject has not been previously treated with another therapeutic agent for the breast cancer. In some embodiments, the subject has not been previously treated with lapatinib, neratinib, afatinib, or capecitabine. In some embodiments, the subject has not been previously treated with lapatinib. In some embodiments, the subject has not been previously treated with neratinib. In some embodiments, the subject has not been previously treated with afatinib. In some embodiments, the subject has not been previously treated with capecitabine. In some embodiments, the subject has not been previously treated with an anti-HER2 antibody-drug conjugate (e.g., ado-trastuzumab emtansine or trastuzumab deruxtecan).

In some embodiments, the subject has not been previously treated with an anti-HER2 and/or an anti-EGFR tyrosine kinase inhibitor. An “anti-HER2 tyrosine kinase inhibitor” and “anti-EGFR tyrosine kinase inhibitor” refers to a therapeutic agent that that exhibits HER2 or EGFR inactivation activity (e.g., inhibiting or decreasing).

In some embodiments, the anti-HER2/EGFR tyrosine kinase inhibitor is selected from the group consisting of tucatinib, lapatinib, neratinib, or afatinib. In some embodiments, the subject has not been previously treated with an anti-HER2 antibody-drug conjugate. In some embodiments, the antibody-drug conjugate is selected from the group consisting of ado-trastuzumab, trastuzumab duocarmazine, or trastuzumab deruxtecan.

In some embodiments, the subject may have not been previously treated with tucatinib. In some embodiments, the subject has not been previously treated with an anthracycline. In some embodiments, the subject was not previously treated with anthracycline selected from the group consisting of doxorubicin, epirubicin, mitoxantrone, idarubicin, liposomal doxorubicin, and combinations thereof.

In some embodiments, ther HER2 status of a sample cell is determined. The determination can be made before treatment (i.e., administration of a combination of tucatinib and an anti-HER2 antibody-drug conjugate (e.g., ado-trastuzumab emtansine or trastuzumab deruxtecan) begins, during treatment, or after treatment has been completed. In some instances, determination of the HER2 status results in a decision to change therapy (e.g., adding an anti-HER2 antibody to the treatment regimen, discontinuing the use of the combination of tucatinib and the anti-HER2 antibody-drug conjugate (e.g., ado-trastuzumab emtansine or trastuzumab deruxtecan)), discontinuing therapy altogether, or switching from another treatment method to a method of the present disclosure).

In some embodiments, the sample cell is determined to be overexpressing or not overexpressing HER2. In particular embodiments, the cell is determined to be HER2 3+, HER2 2+, HER2 1+, or HER2 0 (i.e., HER is not overexpressed).

In some embodiments, the sample cell is a cancer cell. In some instances, the sample cell is obtained from a subject who has cancer. The sample cell can be obtained as a biopsy specimen, by surgical resection, or as a fine needle aspirate (FNA). In some embodiments, the sample cell is a circulating tumor cell (CTC).

HER2 expression can be compared to a reference cell. In some embodiments, the reference cell is a non-cancer cell obtained from the same subject as the sample cell. In other embodiments, the reference cell is a non-cancer cell obtained from a different subject or a population of subjects. In some embodiments, measuring expression of HER2 comprises, for example, determining HER2 gene copy number or amplification, nucleic acid sequencing (e.g., sequencing of genomic DNA or cDNA), measuring mRNA expression, measuring protein abundance, or a combination thereof. HER2 testing methods include immunohistochemistry (IHC), in situ hybridization, fluorescence in situ hybridization (FISH), chromogenic in situ hybridization (CISH), ELISAs, and RNA quantification (e.g., of HER2 expression) using techniques such as RT-PCR and microarray analysis.

In some embodiments, the sample cell is determined to be HER2 positive when HER2 is expressed at a higher level in the sample cell compared to a reference cell. In some embodiments, the cell is determined to be HER2 positive when HER2 is overexpressed at least about 1.5-fold (e.g., about 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or more) compared to a reference cell. In particular embodiments, the cell is determined to be HER2 positive when HER2 is overexpressed at least about 1.5-fold compared to the reference cell.

In some embodiments, the sample cell is determined to be HER2 positive when the FISH or CISH signal ratio is greater than 2. In other embodiments, the sample cell is determined to be HER2 positive when the HER2 gene copy number is greater than 6.

In one embodiment of the methods or uses or product for uses described herein, response to treatment with a combination of tucatinib and an anti-HER2 antibody-drug conjugate as described herein is assessed by measuring the time of progression free survival after administration of the combination of tucatinib and the anti-HER2 antibody-drug conjugate. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the combination of tucatinib and the anti-HER2 antibody-drug conjugate. In some embodiments, the subject exhibits progression-free survival of at least about 6 months after administration of the combination of tucatinib and the anti-HER2 antibody-drug conjugate. In some embodiments, the subject exhibits progression-free survival of at least about one year after administration of the combination of tucatinib and the anti-HER2 antibody-drug conjugate. In some embodiments, the subject exhibits progression-free survival of at least about two years after administration of the combination of tucatinib and the anti-HER2 antibody-drug conjugate. In some embodiments, the subject exhibits progression-free survival of at least about three years after administration of the combination of tucatinib and the anti-HER2 antibody-drug conjugate. In some embodiments, the subject exhibits progression-free survival of at least about four years after administration of the combination of tucatinib and the anti-HER2 antibody-drug conjugate. In some embodiments, the subject exhibits progression-free survival of at least about five years after administration of the combination of tucatinib and the anti-HER2 antibody-drug conjugate. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the combination of tucatinib and the anti-HER2 antibody-drug conjugate. In some embodiments, the subject exhibits progression-free survival of at least 6 months after administration of the combination of tucatinib and the anti-HER2 antibody-drug conjugate. In some embodiments, the subject exhibits progression-free survival of at least one year after administration of the combination of tucatinib and the anti-HER2 antibody-drug conjugate. In some embodiments, the subject exhibits progression-free survival of at least two years after administration of the combination of tucatinib and the anti-HER2 antibody-drug conjugate. In some embodiments, the subject exhibits progression-free survival of at least three years after administration of the combination of tucatinib and the anti-HER2 antibody-drug conjugate. In some embodiments, the subject exhibits progression-free survival of at least four years after administration of the combination of tucatinib and the anti-HER2 antibody-drug conjugate. In some embodiments, the subject exhibits progression-free survival of at least five years after administration of the combination of tucatinib and the anti-HER2 antibody-drug conjugate. In some embodiments, the anti-HER2 antibody-drug conjugate is ado-trastuzumab emtansine or trastuzumab deruxtecan.

In one aspect, provided herein are methods for treating or ameliorating a HER2 positive breast cancer in a subject in need thereof, the method comprising administering to the subject a therapeutically effective amount of a combination therapy comprising tucatinib and an anti-HER2 antibody-drug conjugate (e.g., ado-trastuzumab emtansine or trastuzumab deruxtecan). Also provided herein are methods of treating or ameliorating cancer in a subject in need thereof, the method comprising: (a) identifying the subject as having a HER2 positive breast cancer; and (b) administering to the subject a therapeutically effective amount of a combination therapy comprising tucatinib and an anti-HER2 antibody-drug conjugate (e.g., ado-trastuzumab emtansine or trastuzumab deruxtecan). In some embodiments, the antibody of the anti-HER2 antibody-drug conjugate is trastuzumab or a biosimilar thereof. For example, the antibody of the anti-HER2 antibody-drug conjugate is trastuzumab. In some embodiments, the anti-HER2 antibody-drug conjugate is selected from the group consisting of trastuzumab deruxtecan, ado-trastuzumab emtansine, and combinations thereof. In some embodiments, the anti-HER2 antibody-drug conjugate is trastuzumab deruxtecan. In some embodiments, the anti-HER2 antibody-drug conjugate is ado-trastuzumab emtansine.

In one aspect, provided herein are methods for treating or ameliorating a HER2 positive breast cancer in a subject in need thereof, the method comprising administering to the subject an effective amount of a combination therapy comprising tucatinib and an anti-HER2 antibody-drug conjugate (e.g., trastuzumab deruxtecan or ado-trastuzumab emtansine), wherein following administration of the combination therapy, the subject exhibits progression-free survival of at least 7.5 months following administration of the combination therapy. For example, the subject can exhibit progression-free survival of at least eight months, of at least nine months, or at least ten months following administration of the combination therapy. In some embodiments, a subject can exhibit progression-free survival of 7.5 months, 7.6 months, 7.7 months, 7.8 months, 7.9 months, 8 months, 8.2 months, 8.5 months, 8.8 months, 9.6 months, 9.8 months, and 10 months following administration of the combination therapy.

Also provided herein is a method for treating or ameliorating a HER2 positive breast cancer in a subject in need thereof, the method comprising administering to the subject an effective amount of a combination therapy comprising tucatinib and an anti-HER2 antibody-drug conjugate, wherein the subject exhibits a greater than 40% reduction in the risk of disease progression or death as compared to a subject administered the anti-HER2 antibody-drug conjugate alone. For example, the subject administered the combination therapy comprising tucatinib and the anti-HER2 antibody-drug conjugate exhibits a greater than 45% reduction in the risk of disease progression or death as compared to a subject administered the anti-HER2 antibody-drug conjugate alone. In some embodiments, the subject exhibits a 46% reduction in the risk of disease progression or death. In some embodiments, the anti-HER2 antibody-drug conjugate is ado-trastuzumab emtansine or trastuzumab deruxtecan.

In some embodiments as described herein, following administration of the combination therapy comprising tucatinib and the anti-HER2 antibody-drug conjugate (e.g., ado-trastuzumab emtansine) for nine months, the subject has an estimated progression-free survival rate of greater than 40%. For example, the subject has an estimated progression-free survival of 40.5%, 41%, 42%, 43%, 43.6%, 44%, 44.4%, 45%, 45.8%, 46%, 46.8%, 47%, 47.9%, 48%, 48.2%, 48.8%, 49%, 49.7%, 50%, 50.5%, 51%, 52.4%, 52%, 52.9%, 53%, 54%, or 55%. In some embodiments, the subject has an estimated progression-free survival rate of greater than 45%, greater than 50%, or greater than 55% following administration of the combination therapy comprising tucatinib and the anti-HER2 antibody-drug conjugate (e.g., ado-trastuzumab emtansine) for nine months.

In some embodiments as described herein, following administration of the combination therapy comprising tucatinib and the anti-HER2 antibody-drug conjugate (e.g., ado-trastuzumab emtansine or trastuzumab deruxtecan) for twelve months, the subject has an estimated progression-free survival rate of greater than 25%. For example, the subject has an estimated progression-free survival of 25.4%, 26%, 26.6%, 27%, 27.4%, 28%, 28.6%, 29%, 29.3%, 30%, 30.7%, 31%, 31.5%, 32%, 32.8%, 33%, 33.1%, 34%, 34.4%, 35%, 35.5%, 36%, 36.8%, 37%, 37.3%, 38%, 38.6%, 39.7%, or 40%. In some embodiments, the subject has an estimated progression-free survival rate of greater than 30%, greater than 33%, greater than 35% following administration of the combination therapy comprising tucatinib and the anti-HER2 antibody-drug conjugate (e.g., ado-trastuzumab emtansine or trastuzumab deruxtecan) for twelve months.

In some embodiments as described herein, following administration of the combination therapy comprising tucatinib and the anti-HER2 antibody-drug conjugate (e.g., ado-trastuzumab emtansine or trastuzumab deruxtecan) for fifteen months, the subject has an estimated progression-free survival rate of greater than 20%. For example, the subject has an estimated progression-free survival of 20.2%, 20.5%, 21%, 21.3%, 22%, 22.6%, 23%, 23.7%, 24%, 24.4%, 25%, 25.6%, 26%, 26.2%, 27%, 27.4%, 28%, 28.6%, 29%, 29.3%, 30%, 30.7%, 31%, 31.5%, 32%, 32.8%, 33%, 33.8%, or 34%, In some embodiments, the subject has an estimated progression-free survival rate of greater than 25%, greater than 27%, greater than 30%, or greater than 33% following administration of the combination therapy comprising tucatinib and the anti-HER2 antibody-drug conjugate (e.g., ado-trastuzumab emtansine or trastuzumab deruxtecan) for fifteen months.

In one embodiment of the methods or uses or product for uses described herein, response to treatment with a combination of tucatinib and an anti-HER2 antibody-drug conjugate as described herein is assessed by measuring the time of overall survival after administration of the combination of tucatinib and the anti-HER2 antibody-drug conjugate. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the combination of tucatinib and the anti-HER2 antibody-drug conjugate. In some embodiments, the subject exhibits overall survival of at least about 6 months after administration of the combination of tucatinib and the anti-HER2 antibody-drug conjugate. In some embodiments, the subject exhibits overall survival of at least about one year after administration of the combination of tucatinib and the anti-HER2 antibody-drug conjugate. In some embodiments, the subject exhibits overall survival of at least about two years after administration of the combination of tucatinib and the anti-HER2 antibody-drug conjugate. In some embodiments, the subject exhibits overall survival of at least about three years after administration of the combination of tucatinib and the anti-HER2 antibody-drug conjugate. In some embodiments, the subject exhibits overall survival of at least about four years after administration of the combination of tucatinib and the anti-HER2 antibody-drug conjugate. In some embodiments, the subject exhibits overall survival of at least about five years after administration of the combination of tucatinib and the anti-HER2 antibody-drug conjugate. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least about 12 months, at least eighteen months, at least two years, at least three years, at least four years, or at least five years after administration of the combination of tucatinib and the anti-HER2 antibody-drug conjugate. In some embodiments, the subject exhibits overall survival of at least 6 months after administration of the combination of tucatinib and the anti-HER2 antibody-drug conjugate. In some embodiments, the subject exhibits overall survival of at least one year after administration of the combination of tucatinib and the anti-HER2 antibody-drug conjugate. In some embodiments, the subject exhibits overall survival of at least two years after administration of the combination of tucatinib and the anti-HER2 antibody-drug conjugate. In some embodiments, the subject exhibits overall survival of at least three years after administration of the combination of tucatinib and the anti-HER2 antibody-drug conjugate. In some embodiments, the subject exhibits overall survival of at least four years after administration of the combination of tucatinib and the anti-HER2 antibody-drug conjugate. In some embodiments, the subject exhibits overall survival of at least five years after administration of the combination of tucatinib and the anti-HER2 antibody-drug conjugate. In some embodiments, the anti-HER2 antibody-drug conjugate is ado-trastuzumab emtansine or trastuzumab deruxtecan.

In one aspect, the present disclosure provides methods for treating or ameliorating a HER2 positive breast cancer in a subject in need thereof, the method comprising administering to the subject an effective amount of a combination therapy comprising tucatinib and an anti-HER2 antibody-drug conjugate (e.g., ado-trastuzumab emtansine or trastuzumab deruxtecan), wherein following administration of the combination therapy, the subject exhibits an overall survival of at least eighteen months following administration of the combination therapy. For example, the subject can exhibit an overall survival of at least nineteen months following administration of the combination therapy. In some embodiments, a subject can exhibit an overall survival of 18.2 months, 18.3 months, 18.5 months, 18.8 months, 19 months, 19.2 months, 19.5 months, 19.8 months, 20 months, 20.3 months, 20.6 months, 20.8 months, 21 months, 21.2 months, 21.5 months, 21.9 months, 22 months, 22.4 months, 22.6 months, 22.8 months, 23 months, 23.3 months, 23.6 months, 24 months, 25 months, 26 months, 27 months, 27.5 months, 28 months, 28.5 months, 29 months, 29.5 months, 30 months, 30.5 months, or 31 months.

In one aspect, the present disclosure provides methods for treating or ameliorating a HER2 positive breast cancer in a subject in need thereof, the method consisting essentially of administering to the subject an effective amount of tucatinib and an effective amount of an anti-HER2 antibody-drug conjugate (e.g., ado-trastuzumab emtansine or trastuzumab deruxtecan), wherein following administration of the tucatinib and the ado-trastuzumab emtasine, the subject exhibits an overall survival of at least eighteen months following the administration. For example, the subject can exhibit an overall survival of at least nineteen months following the administration. In some embodiments, a subject can exhibit an overall survival of 18.2 months, 18.3 months, 18.5 months, 18.8 months, 19 months, 19.2 months, 19.5 months, 19.8 months, 20 months, 20.3 months, 20.6 months, 20.8 months, 21 months, 21.2 months, 21.5 months, 21.9 months, 22 months, 22.4 months, 22.6 months, 22.8 months, 23 months, 23.3 months, 23.6 months, 24 months, 25 months, 26 months, 27 months, 27.5 months, 28 months, 28.5 months, 29 months, 29.5 months, 30 months, 30.5 months, or 31 months.

Also provided herein is a method for treating or ameliorating a HER2 positive breast cancer in a subject in need thereof, the method comprising administering to the subject an effective amount of a combination therapy comprising tucatinib and an anti-HER2 antibody-drug conjugate (e.g., ado-trastuzumab emtansine or trastuzumab deruxtecan), wherein the subject exhibits a greater than 30% reduction in the risk of death as compared to a subject administered the anti-HER2 antibody-drug conjugate (e.g., ado-trastuzumab emtansine or trastuzumab deruxtecan) alone. In some embodiments, the subject exhibits a 34% reduction in the risk of death.

In some embodiments as described herein, following administration of the combination therapy comprising tucatinib and the anti-HER2 antibody-drug conjugate (e.g., ado-trastuzumab emtansine or trastuzumab deruxtecan) for twenty-four months, the subject has an estimated overall survival rate of greater than 35%. For example, the subject has an estimated overall survival of 35.4%, 35.5%, 36%, 36.6%, 37%, 37.3%, 38%, 38.6%, 39.7%, 40%, 40.5%, 41%, 42%, 43%, 43.6%, 44%, 44.4%, 45%, 45.8%, 46%, 46.8%, 47%, 47.9%, 48%, 48.2%, 48.8%, 49%, 49.7%, 50%, 50.5%, 51%, 52.4%, 52%, 52.8%, or 53%. In some embodiments, the subject has an estimated overall survival rate of greater than 40%, greater than 44%, greater than 50%, or greater than 52% following administration of the combination therapy comprising tucatinib and the anti-HER2 antibody-drug conjugate (e.g., ado-trastuzumab emtansine or trastuzumab deruxtecan) for twenty-four months.

In some embodiments as described herein, following administration of the combination therapy comprising tucatinib and the anti-HER2 antibody-drug conjugate (e.g., ado-trastuzumab emtansine or trastuzumab deruxtecan) for thirty months, the subject has an estimated overall survival rate of greater than 30%. For example, the subject has an estimated overall survival of 30.7%, 31%, 31.5%, 32%, 32.8%, 33%, 33.8%, 34%, 34.6%, 35.4%, 35.5%, 36%, 36.6%, 37%, 37.3%, 38%, 38.6%, 39.7%, 40%, 40.5%, 41%, 42%, 42.8%, 43%, 43.6%, 44%, 44.4%, 45%, 45.8%, 46%, 46.8%, 47%, 47.9%, 48%, 48.2%, 48.8%, 49%, 49.7%, 50%, 50.5%, 51%, 51.3%, or 52%. In some embodiments, the subject has an estimated overall survival rate of greater than 35%, greater than 40%, greater than 42%, or greater than 50% following administration of the combination therapy comprising tucatinib and the anti-HER2 antibody-drug conjugate (e.g., ado-trastuzumab emtansine or trastuzumab deruxtecan) for thirty months.

Further provided herein is a method of treating or ameliorating a brain metastasis in a subject having HER2 positive breast cancer, the method comprising administering to the subject an effective amount of a combination therapy comprising tucatinib and an anti-HER2 antibody-drug conjugate (e.g., ado-trastuzumab emtansine or trastuzumab deruxtecan). In some embodiments, the time to additional intervention (e.g., radiation, surgery, or a combination thereof) for treatment of the brain metastasis in the subject has been increased. In some embodiments, the time to additional intervention is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, and at least 99%. In some embodiments, the time to additional intervention is increased by at least one week, two weeks, three weeks, at least one month, at least two months, at least three months, at least four months, at least five months, at least six months, at least seven months, at least eight months, at least nine months, at least ten months, at least eleven months, at least twelve months, at least eighteen months, and at least twenty-four months. In some embodiments, the need for additional intervention for treatment of the brain metastasis in the subject has been prevented. In some embodiments, the increase in time to additional intervention is compared to a subject administered the anti-HER2 antibody-drug conjugate (e.g., ado-trastuzumab emtansine or trastuzumab deruxtecan) alone over the same period of time.

In some embodiments, of the methods or uses or product for uses described herein, response to treatment with a combination of tucatinib and an anti-HER2 antibody-drug conjugate (e.g., ado-trastuzumab emtansine or trastuzumab deruxtecan) as described herein, results in prevention of the development of a brain metastasis in the subject (e.g., in a subject that did not previously develop brain metastasis). In some embodiments, of the methods or uses or product for uses described herein, response to treatment with a combination of tucatinib and an anti-HER2 antibody-drug conjugate (e.g., ado-trastuzumab emtansine or trastuzumab deruxtecan) as described herein, prevents the development of new brain metastasis (e.g., in a subject previously identified as having brain metastasis). In some embodiments, regression of an existing brain metastasis in the subject has been promoted. In some embodiments, the size of an existing brain metastasis in the subject has been reduced.

Also provided herein is a method for treating or ameliorating a HER2 positive breast cancer in a subject in need thereof, wherein the subject has a brain metastasis, the method comprising administering to the subject an effective amount of a combination therapy comprising tucatinib and an anti-HER2 antibody-drug conjugate (e.g., ado-trastuzumab emtansine or trastuzumab deruxtecan), wherein the subject exhibits a greater than 50% reduction in the risk of disease progression or death as compared to a subject administered the anti-HER2 antibody-drug conjugate (e.g., ado-trastuzumab emtansine or trastuzumab deruxtecan) alone. In some embodiments, the subject exhibits a 52% reduction in the risk of disease progression or death.

In one aspect, provided herein are methods for treating or ameliorating a HER2 positive breast cancer in a subject in need thereof, wherein the subject has a brain metastasis, the method comprising administering to the subject an effective amount of a combination therapy comprising tucatinib and an anti-HER2 antibody-drug conjugate (e.g., ado-trastuzumab emtansine or trastuzumab deruxtecan), wherein following administration of the combination therapy, the subject exhibits progression-free survival of at least six months following administration of the combination therapy. For example, the subject can exhibit progression-free survival of at least seven months, at least eight months, at least nine months, or at least ten months following administration of the combination therapy. In some embodiments, a subject can exhibit progression-free survival of 6.2 months, 6.4 months, 6.9 months, 7 months, 7.5 months, 7.6 months, 7.7 months, 7.8 months, 7.9 months, 8 months, 8.2 months, 8.5 months, 8.8 months, 9.5 months, 9.8 months, and 10 months following administration of the combination therapy.

In some embodiments as described herein, following administration of the combination therapy comprising tucatinib and the anti-HER2 antibody-drug conjugate (e.g., ado-trastuzumab emtansine or trastuzumab deruxtecan) for nine months, the subject having brain metastasis has an estimated progression-free survival rate of greater than 30%. For example, the subject has an estimated progression-free survival of 30.7%, 31%, 31.5%, 32%, 32.8%, 33%, 33.1%, 34%, 34.9%, 35%, 35.5%, 36%, 36.8%, 37%, 37.3%, 38%, 38.6%, 39.7%, 40%, 40.5%, 41%, 42%, 43%, 43.4%, 44%, 44.4%, 45%, 45.8%, 46%, 46.8%, 47%, 47.9%, 48%, 48.2%, 48.8%, 49%, 49.7%, 50%, 50.5%, 51%, 51.5%, or 52%. In some embodiments, the subject has an estimated progression-free survival rate of greater than 40%, greater than 45%, or greater than 50% following administration of the combination therapy comprising tucatinib and the anti-HER2 antibody-drug conjugate (e.g., ado-trastuzumab emtansine or trastuzumab deruxtecan) for nine months.

In some embodiments as described herein, following administration of the combination therapy comprising tucatinib and the anti-HER2 antibody-drug conjugate (e.g., ado-trastuzumab emtansine or trastuzumab deruxtecan) for twelve months, the subject having a brain metastasis has an estimated progression-free survival rate of greater than 15%. For example, the subject has an estimated progression-free survival of 15.8%, 16%, 16.5%, 17%, 18%, 18.8%, 19%, 20%, 22%, 23.3%, 24.9%, 25%, 25.4%, 26%, 26.6%, 27%, 27.4%, 28%, 28.6%, 29%, 29.3%, 30%, 30.7%, 31%, 31.5%, 32%, 32.8%, 33%, 33.1%, 34%, 34.3%, or 35%. In some embodiments, the subject has an estimated progression-free survival rate of greater than 20%, greater than 25%, greater than 30%, or greater than 34% following administration of the combination therapy comprising tucatinib and the anti-HER2 antibody-drug conjugate (e.g., ado-trastuzumab emtansine or trastuzumab deruxtecan) for twelve months.

In some embodiments, the methods provided herein further comprise administration of an anti-diarrheal agent in a subject having a HER2 positive breast cancer and being treated with an effective amount of a combination therapy comprising tucatinib and an anti-HER2 antibody-drug conjugate. In some embodiments, the methods provided herein further comprise treating a HER2 positive breast cancer in a subject in need thereof, the method comprising: (a) administering to the subject a therapeutically effective amount of a combination therapy comprising tucatinib and an anti-HER2 antibody-drug conjugate (e.g., trastuzumab deruxtecan or ado-trastuzumab emtansine) and (b) administering an effective amount of an anti-diarrheal agent.

For example, the anti-diarrheal agent can be administered prophylactically (e.g., before or concurrently with administration of the combination therapy comprising tucatinib and the anti-HER2 antibody-drug conjugate and/or before the subject has symptoms of diarrhea), reactively (e.g., after administration of the combination therapy comprising tucatinib and the anti-HER2 antibody-drug conjugate and following at least one episode of diarrhea), or a combination thereof. In some embodiments, the anti-diarrheal agent is administered to reduce the severity or incidents of diarrhea, or to prevent diarrhea. In some embodiments, the anti-diarrheal agent reduces the likelihood of a subject developing diarrhea. In some embodiments, the anti-HER2 antibody-drug conjugate is ado-trastuzumab emtansine. In some embodiments, the anti-HER2 antibody-drug conjugate is trastuzumab deruxtecan.

In some embodiments, the methods provided herein further comprise reducing the severity or incidence of incidents of diarrhea, or preventing diarrhea in a subject having a HER2 positive breast cancer and being treated with an effective amount of a combination therapy comprising tucatinib and an anti-HER2 antibody-drug conjugate (e.g., trastuzumab deruxtecan or ado-trastuzumab emtansine), the method comprising administering an effective amount of an anti-diarrheal agent prophylactically. In some embodiments, the methods provided herein further comprise reducing the likelihood of a subject developing diarrhea, wherein the subject has a HER2 positive breast cancer and is being treated with an effective amount of a combination therapy comprising tucatinib and an anti-HER2 antibody-drug conjugate (e.g., trastuzumab deruxtecan or ado-trastuzumab emtansine), the method comprising administering an effective amount of an anti-diarrheal agent prophylactically.

In some embodiments, the combination therapy and the anti-diarrheal agent are administered sequentially. In some embodiments, the combination therapy and the anti-diarrheal agent are administered concurrently. In some embodiments, the anti-diarrheal agent is administered prior to administration of the combination therapy. For example, one hour before, two hours before, four hours before, six hours before, twelve hours before, one day before, two days before, three days before, four days before, five days before, or one week before. In some cases, the subject is exhibiting symptoms of diarrhea prior to administration of the anti-diarrheal agent. In other cases, the subject is not exhibiting symptoms of diarrhea prior to administration of the anti-diarrheal agent.

Non-limiting examples of anti-diarrheal agents include loperamide, budesonide (e.g., in combination with loperamide), prophylactic antibiotics (e.g., doxycycline), probiotics, electrolyte replacement solutions, colestipol, colestipol in combination with loperamide, octreotide, crofelemer, TJ14, Bacillus Cereus, calcium aluminosilicate, sulfasalazine, cefpodoxime, elsiglutide, glutamine, codeine, diphenoxylate, atropine, bismuth subsalicylate, diphenoxylate, atropine, attapulgite, activated charcoal, bentonite, saccharomyces boulardii lyo, rifaximin, neomycin, alosetron, octreotide, crofelemer, opium, cholestyramine, and colesevelam.

In some embodiments, the methods provided herein further comprise administration of an antiemetic agent in a subject having a HER2 positive breast cancer and being treated with an effective amount of a combination therapy comprising tucatinib and an anti-HER2 antibody-drug conjugate. For example, the antiemetic agent can be administered prophylactically (e.g., before or concurrently with administration of the combination therapy comprising tucatinib and the anti-HER2 antibody-drug conjugate and/or before the subject has symptoms of nausea), reactively (e.g., after administration of the combination therapy comprising tucatinib and the anti-HER2 antibody-drug conjugate and following at least one episode of nausea), or a combination thereof. In some embodiments, the antiemetic agent is administered to reduce the severity or incidents of nausea, or to prevent nausea. In some embodiments, the antiemetic agent reduces the likelihood of a subject developing nausea. In some embodiments, the anti-HER2 antibody-drug conjugate is ado-trastuzumab emtansine. In some embodiments, the anti-HER2 antibody-drug conjugate is trastuzumab deruxtecan.

In some embodiments, the combination therapy and the antiemetic agent are administered sequentially. In some embodiments, the combination therapy and the antiemetic agent are administered concurrently. In some embodiments, the antiemetic agent is administered prior to administration of the combination therapy. For example, one hour before, two hours before, four hours before, six hours before, twelve hours before, one day before, two days before, three days before, four days before, five days before, or one week before. In some cases, the subject is exhibiting symptoms of nausea prior to administration of the antiemetic agent. In other cases, the subject is not exhibiting symptoms of nausea prior to administration of the antiemetic agent.

Non-limiting examples of antiemetic agents include a 5-HT3 receptor antagonist such as dolasetron, granisetron, ondansetron, tropisetron; and palonosetron; a dopamine antagonist such as domperidone, olanzapine, haloperidol, alizapride, prochlorperazine, chlorpromazine, and metoclopramide; a NK1 receptor antagonist such as aprepitant, casopitant, and rolapitant; an antihistamine such as cinnarizine, cyclizine, diphenhydramine, dimenhydrinate, doxylamine, mirtazapine, meclizine, promethazine, and hydroxyzine; cannabinoids such as cannabis, dronabionl, synthetic cannabinoids such as nabilone, and sativex; benzodiazepines such as midazolam and laorazepam; anticholinergics such as scopolamine, atropine, and diphenhydramine; steroids such as dexamethasone; timethobenzamide, ginger, emtrol, propofol, peppermint, muscimol, bismuth-subsalicylate, vitamin B-6, and ajwain.

In some embodiments, of any of the methods described herein, the administration of the tucatinib, or a salt or solvate thereof, changes the overall amount of HER2 in a solid tumor. As used herein, the term “overall” refers to the amount of protein measurable by an immunoblot assay. In some embodiments, the administration of the tucatinib, or a salt or solvate thereof, increases the overall amount of HER2 in a solid tumor. In some embodiments, the overall amount of HER2 in the solid tumor is determined by an immunoblot assay. In some embodiments, the immunoblot assay is a western blot analysis. In some embodiments, the overall amount of HER2 in the solid tumor is determined by western blot analysis.

In some embodiments, of any of the methods described herein, the administration of the tucatinib, or salt or solvate thereof, changes the amount of membrane-bound HER2 in the solid tumor. As used herein, the term “membrane-bound” refers to protein that is connected to a cell-surface. In some embodiments, the administration of the tucatinib, or salt or solvate thereof, increases the amount of membrane-bound HER2 in the solid tumor. In some embodiments, the amount of membrane-bound HER2 in the solid tumor is determined by flow cytometry. IN some embodiments, the flow cytometry utilizes a labeling method selected from the group consisting of fluorescent labeling, quantum dots, and isotope labeling. In some embodiments the flow cytometry used for the detection of membrane-bound HER2 is fluorescence-activated cell sorting (FACS). In some embodiments, the amount of membrane-bound HER2 in the solid tumor is determined by quantitative fluorescence activated cell sorting (qFACS).

In some embodiments, of any of the methods described herein, the administration of the tucatinib, or a salt or solvate thereof, changes the dwell time of HER2 at the cell surface. As used herein, the term “dwell time” refers to an amount of time that a protein positions at the surface of the cell. In some embodiments of any of the methods described herein, the administration of the tucatinib, or a salt or solvate thereof, increases the dwell time of HER2 at the cell surface. In some embodiments of any of the methods described herein, the administration of tucatinib, or salt or solvate thereof, changes an internalization of membrane-bound HER2. In some embodiments of any of the methods described herein, the administration of tucatinib, or salt or solvate thereof, increases an internalization of membrane-bound HER2. In some embodiments of any of the methods described herein, the administration of the tucatinib, or salt or solvate thereof, changes a lysosomal degradation of HER2. In some embodiments of any of the methods described herein, the administration of the tucatinib, or salt or solvate thereof, increases a lysosomal degradation of HER2.

C. Tucatinib Dose and Administration

In some embodiments, a dose of tucatinib is between about 0.1 mg and 10 mg/kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg/kg of the subject's body weight). In other embodiments, a dose of tucatinib is between about 10 mg and 100 mg/kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg/kg of the subject's body weight). In some embodiments, a dose of tucatinib is at least about 100 mg to 500 mg/kg of the subject's body weight (e.g., at least about 100, 125, 150, 175, 200, 225, 250, 275, 300, 325, 350, 375, 400, 425, 450, 475, or 500 mg/kg of the subject's body weight). In particular embodiments, a dose of tucatinib is between about 1 mg and 50 mg/kg of the subject's body weight (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/kg of the subject's body weight). In some instances, a dose of tucatinib is about 50 mg/kg of the subject's body weight.

In some embodiments, a dose of tucatinib comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of tucatinib. In other embodiments, a dose of tucatinib comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of tucatinib. In particular embodiments, a dose of tucatinib is about 300 mg (e.g., when administered twice per day). In certain of these embodiments, a dose of tucatinib is 300 mg (e.g., 6×50 mg tablets; or 2×150 mg tablets), administered twice per day.

In some embodiments, a dose of tucatinib comprises at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of tucatinib.

In some embodiments, a dose of tucatinib, or salt or solvate thereof, contains a therapeutically effective amount of tucatinib, or salt or solvate thereof. In other embodiments, a dose of tucatinib, or salt or solvate thereof, contains less than a therapeutically effective amount of tucatinib, or salt or solvate thereof, (e.g., when multiple doses are given in order to achieve the desired clinical or therapeutic effect).

Tucatinib, or salt or solvate thereof, can be administered by any suitable route and mode. Suitable routes of administering antibodies and/or antibody-drug conjugate of the present disclosure are well known in the art and may be selected by those of ordinary skill in the art. In one embodiment, tucatinib administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of tucatinib is intravenous injection or infusion. In some embodiments, the route of administration of tucatinib is intravenous infusion. In some embodiments, the route of administration of tucatinib is intravenous injection or infusion. In some embodiments, the tucatinib is intravenous infusion. In some embodiments, the route of administration of tucatinib is oral.

In one embodiment of the methods or uses or product for uses provided herein, tucatinib is administered to the subject daily, twice daily, three times daily or four times daily. In some embodiments, tucatinib is administered to the subject every other day, once about every week or once about every three weeks. In some embodiments, tucatinib is administered to the subject once per day. In some embodiments, tucatinib is administered to the subject twice per day. In some embodiments, tucatinib is administered to the subject at a dose of about 300 mg twice per day. In some embodiments, tucatinib is administered to the subject at a dose of 300 mg twice per day. In some embodiments, tucatinib is administered to the subject at a dose of about 600 mg once per day. In some embodiments, tucatinib is administered to the subject at a dose of 600 mg once per day. In some embodiments, tucatinib is administered to the subject twice per day on each day of a 21 day treatment cycle. In some embodiments, the tucatinib is administered to the subject orally.

E. Anti-HER2 Antibody-Drug Conjugate

In some embodiments, the antibody of the anti-HER2 antibody-drug conjugate is a monoclonal antibody. Non-limiting examples of anti-HER2 monoclonal antibodies can include: trastuzumab, pertuzumab, MGAH22, MCLA-128, ZW25, GBR1302, and PRS-343.

In some embodiments, the anti-HER2 antibody-drug conjugate is a trastuzumab-drug conjugate. Trastuzumab (CAS 180288-69-1) is an anti-HER2 monoclonal antibody used to treat breast cancer and sold under various tradenames including HERCEPTIN, OGIVRI, and HERZUMA. As used herein, “trastuzumab” also includes biosimilars as defined herein. Trastuzumab can have a sequence and/or can bind a HER2 antigen as described in U.S. Pat. Nos. 5,677,171; 5,821,337; 6,054,297; 6,165,464; 6,339,142; 6,407,213; 6,639,055; 6,719,971; 6,800,738; 7,074,404; Coussens et al (1985) Science 230:1132-9; Slamon et al (1989) Science 244:707-12; and Slamon et al (2001) New Engl. J. Med. 344:783-792), each of which is incorporated herein by reference in its entirety.

The term “drug loading” refers to the average number of drug moieties per antibody in the anti-HER2 antibody-drug conjugate. In some embodiments of the anti-HER2 antibody-drug conjugate described herein, drug loading (i.e., the average number of drug moieties per antibody) may range from 1 to 8 drugs (D) per antibody (Ab), i.e. where 1, 2, 3, 4, 5, 6, 7, and 8 drug moieties are covalently attached to the antibody. Compositions of ADC include collections of antibodies conjugated with a range of drugs, from 1 to 8. The average number of drugs per antibody in preparations of ADC from conjugation reactions may be characterized by conventional means such as mass spectroscopy, ELISA assay, electrophoresis, and HPLC (e.g., by methods described in U.S. Pat. No. 10,124,069, which is incorporated herein by reference in its entirety).

Each drug moiety of the anti-HER2 antibody-drug conjugate (e.g., trastuzumab-drug conjugate) can be a chemotherapeutic agent. As used herein, a chemotherapeutic agent is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, and topoisomerase inhibitors. In some embodiments, each drug moiety of the anti-HER2 antibody-drug conjugates described herein can be a cytotoxic agent. Cytotoxic agents include any agent that is detrimental to the growth, viability or propagation of cells, including, but not limited to, tubulin-interacting agents and DNA-damaging agents. Non-limiting examples of cytotoxic agents include: e.g., 1-(2-chloroethyl)-1,2-dimethanesulfonyl hydrazide, 1,8-dihydroxy-bicyclo[7.3.1]trideca-4,9-diene-2,6-diyne-13-one, 1-dehydrotestosterone, 5-fluorouracil, 6-mercaptopurine, 6-thioguanine, 9-amino camptothecin, actinomycin D, amanitins, aminopterin, anguidine, anthracycline, anthramycin (AMC), auristatins, bleomycin, busulfan, butyric acid, calicheamicins, camptothecin, carminomycins, carmustine, cemadotins, cisplatin, colchicin, combretastatins, cyclophosphamide, cytarabine, cytochalasin B, dactinomycin, daunorubicin, decarbazine, di acetoxypentyldoxorubicin, dibromomannitol, dihydroxy anthracin dione, disorazoles, dolastatin (e.g., dolastatin 10), doxorubicin, duocarmycin, echinomycins, eleutherobins, emetine, epothilones, esperamicin, estramustines, ethidium bromide, etoposide, fluorouracils, geldanamycins, gramicidin D, glucocorticoids, irinotecans, kinesin spindle protein (KSP) inhibitors, leptomycins, leurosines, lidocaine, lomustine (CCNU), maytansinoids, mechlorethamine, melphalan, mercatopurines, methopterins, methotrexate, mithramycin, mitomycin, mitoxantrone, N8-acetyl spermidine, podophyllotoxins, procaine, propranolol, pteridines, puromycin, pyrrolobenzodiazepines (PBDs), rhizoxins, streptozotocin, tallysomycins, taxol, tenoposide, tetracaine, thioepa chlorambucil, tomaymycins, topotecans, tubulysin, vinblastine, vincristine, vindesine, vinorelbines, and derivatives of any of the foregoing.

In certain embodiments, the cytotoxic agent is selected from the group consisting of: tubulin monomer polymerizing inhibitors (e.g., auristatin derivatives such as MMAE and MMAF), microtubule depolymerizing agents (e.g., maytansine derivatives such as DM1 and DM4), DNA-binding agents (e.g., duocarmycin, pyrrolibenzodiazepines (PBDA)), topoisomerase inhibitors (e.g., doxorubicin and daunorubicin), vinca alkaloids (e.g., vinblastine), and DNA minor-groove binding agents (e.g., calicheamicin). In certain embodiments, the cytotoxic agent is selected from the group consisting of: an auristatin, a maytansinoid, a tubulysin, a tomaymycin, calicheamicin, a camptothecin derivative, and a dolastatin derivative. In certain embodiments, the cytotoxic agent is an auristatin selected from MMAE, MMA, and MMAF. In certain embodiments, the cytotoxic agent is a maytansinoid selected from DM1 and DM4. Further maytansinoid derivatives and methods of making and using the same are described in WO 2019/212965; WO 2014/145090; WO 2015/031396; US 2016/0375147; US 2017/0209591; U.S. Pat. Nos. 10,124,069; 7,276,497; 6,913,748; 6,441,163; U.S. Pat. No. 633,410 (RE39151); U.S. Pat. No. 5,208,020; Widdison et al (2006) J. Med. Chem. 49:4392-4408; Chari et al (1992) Cancer Res. 52:127-131; Liu et al (1996) Proc. Natl. Acad. Sci USA 93:8618-8623, each of which is incorporated herein by reference in its entirety. In certain embodiments, the cytotoxic agent is a camptothecin or derivative thereof (e.g., exatecan). Further examples of campothecin derivatives are described in Mol Pharm. 2010; 7(2): 307-349; and Am J Cancer Res. 2017; 7(12): 2350-2394, each of which is incorporated herein by reference in its entirety.

In some embodiments, the drug moiety of the anti-HER2 antibody-drug conjugate is covalently attached to the anti-HER2 antibody via a linker. In certain of these embodiments, the linker can be as defined in WO 2019/212965; U.S. Pat. Nos. 10,087,260; and 9,504,756, each of which is incorporated herein by reference in its entirety.

Non-limiting examples of HER2 directed antibody-drug conjugates include: (1) trastuzumab deruxtecan (DS-8201a) (Iwata et al., Mol. Cancer Ther., 17(7) 1494-503 (2018) (an ADC composed of trastuzumab, an enzymatically cleavable maleimide glycynglycyn-phenylalanyn-glycyn (GGFG) peptide linker and a topoisomerase I inhibitor)); (2) trastuzumab vc-seco-DUBA (SYD985) (Dokter et al., Mol. Cancer Ther., 13(11): doi: 10.1158/1535-7163.MCT-14-0040-T (2014) (a monoclonal HER2 directed antibody trastuzumab linked via a cleavable valine-citrulline peptide to the synthetic duocarmycin analogon seco-DUocarmycin-hydroxyBenzamide-Azaindole (vc-seco-DUBA)); (3) ado-trastuzumab emtansine (T-DM1) (U.S. Pat. No. 8,337,856; Lambert et al., J. Med. Chem., 28(57): 6949-64 (2014)); (4) A166 (Klus Pharma, Inc) (a monoclonal anti-HER2 antibody conjugated to a cytotoxic agent); (5) Alt-P7 (Doronina et al., Nat. Biotechnol., 21: 778-784 (2003) (an ADC composed of the trastuzumab biobetter HM2 conjugated in a site-specific manner to monomethyl auristatin E (MMAE); (6) ARX-788 (Humphreys et al., Cancer Res., 75: 369 (2015) (a monoclonal HER2 targeting antibody site-specifically conjugated, via a non-natural amino acid linker para-acetyl-phenylalanine (pAcF), to monomethyl auristatin F (MMAF))); (7) DHES0815A (Rinnerthaler et al., Int. J. Mol. Sci., 20(5): 1115 (2019) (a monoclonal HER-2 targeting antibody linked to pyrrolo[2,1-c][1,4]benzodiazepine monoamide (PBD-MA)); (8) MEDI4276 (Li et al., Cancer Cell, 29:117-129 (2016) (a nADC composed of a HER2-bispecific antibody targeting two different epitopes on HER2, site-specifically conjugated via a maleimidocaproyl linker to the potent tubulysin-based microtubule inhibitor AZ13599185)); (9) XMT-1522 (Bergstrom et al., Cancer Res., 76 (2016) (an ADC composed of a novel IgG1 anti-HER2 monoclonal antibody (HT-19) conjugated with the Dolaflexin® platform to auristatin-based drug payload molecules (Auristatin F-hydroxypropylamide, AF-HPA))); and (10) RC48 (Yao et al., Breast Cancer Res. Treat., 153: 123-133 (2015) (a humanized anti-HER2 antibody hertuzumab conjugated with monomethyl auristatin E (MMAE) via a cleavable linker)).

In some embodiments, the anti-HER2 antibody-drug conjugate is a selected from the group consisting of ado-trastuzumab emtansine, trastuzumab vc-seco-DUBA (SYD985), copper Cu 64-DOTA-trastuzumab, trastuzumab deruxtecan (DS-8201a), and (vic-) trastuzumab duocarmazine. In certain embodiments, the anti-HER2 antibody-drug conjugate is ado-trastuzumab emtansine. In certain embodiments, the anti-HER2 antibody-drug conjugate is trastuzumab deruxtecan (DS-8201a). In some embodiments, the anti-HER2 antibody-drug conjugate is selected from the group consisting of: XMT-1522; RC-48; ALT-P7 (HM2-MMAE); ARX788; DHES0815A; MEDI4276; ADCT-502; and ertumaxomab.

Further examples of anti-HER2 antibody-drug conjugates that can be used in one or more methods provided herein include those described in U.S. Pat. Nos. 9,345,661; 7,879,325; 9,518,118; 8,337,856; 7,575,748; 8,309,300; 8,652,479; 9,243,069; Iwata et al., Mol. Cancer Ther., 17(7) 1494-503 (2018); Dokter et al., Mol. Cancer Ther., 13(11): doi: 10.1158/1535-7163.MCT-14-0040-T (2014); Chan et al., EJNMMI Res., 1(15): doi: 10.1186/2191-219X-1-15 (2011); Lambert et al., J. Med. Chem., 28(57): 6949-64 (2014); Rinnerthaler et al., Int. J Mol. Sci., 20(5): 1115 (2019); and Banerji et al., Lancet, 20(8): P1124-1135 (2019) DOI:https://doi.org/10.1016/S1470-2045(19)30328-6, each of which is incorporated herein by reference in its entirety. Additional non-limiting examples of HER2 antibody-drug conjugates include: U.S. Pat. Nos. 10,160,812; 9,738,726; 10,092,659; 10,118,972; 10,155,821; 10,160,812; 8,663,643; U.S. Patent Application Publication No. 2019/0330368; and U.S. Application Publication No. 2019/0077880, each of which is incorporated herein by reference in its entirety.

In some embodiments, a dose of the anti-HER2 antibody-drug conjugate is between about 0.1 mg and 10 mg/kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg/kg of the subject's body weight). In some embodiments, a dose of the anti-HER2 antibody-drug conjugate is between about 1 mg and 7 mg/kg of the subject's body weight. In some embodiments, a dose of the anti-HER2 antibody-drug conjugate is between 2 mg and 6 mg/kg of the subject's body weight.

In some embodiments, a dose of the anti-HER2 antibody-drug conjugate is about 3 mg/kg of the subject's body weight. In some embodiments, a dose of the anti-HER2 antibody-drug conjugate is about 5 mg/kg of the subject's body weight. In some embodiments, the anti-HER2 antibody-drug conjugate is ado-trastuzumab emtansine. In some embodiments, the anti-HER2 antibody-drug conjugate is trastuzumab deruxtecan. In some embodiments, a dose of ado-trastuzumab emtansine is about 3.6 mg per kg of the subject's body weight.

In some embodiments, a dose of the anti-HER2 antibody-drug conjugate is about 4 mg/kg of the subject's body weight for the first dose of the anti-HER2 antibody-drug conjugate administered to the subject followed by subsequent doses of about 3 mg/kg of the subject's body weight. In some embodiments, a dose of the anti-HER2 antibody-drug conjugate is 4 mg/kg of the subject's body weight for the first dose of the anti-HER2 antibody-drug conjugate administered to the subject followed by subsequent doses of 3.5 mg/kg of the subject's body weight.

In some embodiments, a dose of trastuzumab deruxtecan is about 5.4 mg/kg of the subject's body weight. In some embodiments, a dose of trastuzumab deruxtecan is about 5.4 mg/kg of the subject's body weight for the first dose of trastuzumab deruxtecan administered to the subject followed by subsequent doses of about 4.4 mg/kg of the subject's body weight. In some embodiments, a dose of the trastuzumab deruxtecan is 4.4 mg/kg of the subject's body weight. In some embodiments, a dose of trastuzumab deruxtecan is 3.2 mg/kg of the subject's body weight. In some embodiments, trastuzumab deruxtecan is 4.4 mg/kg of the subject's body weight for the first dose of trastuzumab deruxtecan administered to the subject followed by subsequent doses of 3.2 mg/kg of the subject's body weight.

In some embodiments, a dose of the anti-HER2 antibody-drug conjugate contains a therapeutically effective amount of the anti-HER2 antibody-drug conjugate. In other embodiments, a dose of the anti-HER2 antibody-drug conjugate contains less than a therapeutically effective amount of the anti-HER2 antibody-drug conjugate (e.g., when multiple doses are given in order to achieve the desired clinical or therapeutic effect). In some embodiments, the anti-HER2 antibody-drug conjugate is administered to the subject once about every 1 to 4 weeks. In certain embodiments, the anti-HER2 antibody-drug conjugate is administered once about every 1 week, once about every 2 weeks, once about every 3 weeks or once about every 4 weeks. In one embodiment, the anti-HER2 antibody-drug conjugate is administered once about every 3 weeks. In some embodiments, the anti-HER2 antibody-drug conjugate is administered to the subject once every 1 to 4 weeks. In certain embodiments, the anti-HER2 antibody-drug conjugate is administered once every 1 week, once about every 2 weeks, once about every 3 weeks or once about every 4 weeks. In one embodiment, the anti-HER2 antibody-drug conjugate is administered once every 3 weeks.

In some embodiments, the anti-HER2 antibody-drug conjugate (e.g., trastuzumab deruxtecan or ado-trastuzumab emtansine) is administered to the subject subcutaneously. In some embodiments, the anti-HER2 antibody-drug conjugate (e.g., trastuzumab deruxtecan or ado-trastuzumab emtansine) is administered to the subject intravenously. In some embodiments, trastuzumab deruxtecan is administered at a dose of about 5.4 mg/kg once about every 3 weeks to the subject intravenously. In some embodiments, trastuzumab deruxtecan is administered at a dose of about 5.4 mg/kg for the first dose, followed by subsequent doses of about 4.4 mg/kg, where the trastuzumab deruxtecan is administered intravenously. In some embodiments, trastuzumab deruxtecan is administered at a dose of about 4.4 mg/kg, followed by subsequent doses of about 3.2 mg/kg, where the trastuzumab deruxtecan is administered intravenously. In some embodiments, the trastuzumab deruxtecan is administered at a dose of 4.4 mg/kg once every 3 weeks intravenously. In some embodiments, the trastuzumab deruxtecan is administered at a dose of 3.2 mg/kg once every 3 weeks intravenously.

In some embodiments, the anti-HER2 antibody-drug conjugate (e.g., trastuzumab deruxtecan or ado-trastuzumab emtansine) is administered at a dose of about 250 mg once about every 3 weeks and the anti-HER2 antibody-drug conjugate is administered subcutaneously. In some embodiments, the anti-HER2 antibody-drug conjugate is administered at a dose of 250 mg once every 3 weeks and the anti-HER2 antibody-drug conjugate is administered subcutaneously. In some embodiments, the anti-HER2 antibody-drug conjugate is administered at a dose of about 3 mg/kg once about every 3 weeks and the anti-HER2 antibody-drug conjugate is administered intravenously. In some embodiments, the anti-HER2 antibody-drug conjugate is administered at a dose of about 3.6 mg/kg once about every 3 weeks and the anti-HER2 antibody-drug conjugate is administered intravenously. In some embodiments, the anti-HER2 antibody-drug conjugate is administered once about every 3 weeks at a dose of about 4 mg/kg for the first dose of the anti-HER2 antibody-drug conjugate administered to the subject followed by subsequent doses of about 3.5 mg/kg, wherein the anti-HER2 antibody-drug conjugate is administered intravenously. In some embodiments, the anti-HER2 antibody-drug conjugate is administered at a dose of 3.6 mg/kg once every 3 weeks and the anti-HER2 antibody-drug conjugate is administered intravenously. In some embodiments, the anti-HER2 antibody-drug conjugate is administered to the subject on a 21 day treatment cycle and is administered to the subject once per treatment cycle.

In some embodiments, the anti-HER2 antibody-drug conjugate is administered once about every week at a dose of about 3.6 mg/kg, wherein the anti-HER2 antibody-drug conjugate is administered intravenously. In certain embodiments (when administration of the anti-HER2 antibody-drug conjugate has been delayed in a 21-day treatment cycle), the anti-HER2 antibody-drug conjugate is administered once about every week at a dose of about 3.6 mg/kg until resynchronization of the cycle length to 21 days, wherein the anti-HER2 antibody-drug conjugate is administered intravenously. In certain embodiments (when administration of the anti-HER2 antibody-drug conjugate has been delayed in a 21 day treatment cycle), the anti-HER2 antibody-drug conjugate is administered once every week at a dose of 3.6 mg/kg until resynchronization of the cycle length to 21 days, wherein the anti-HER2 antibody-drug conjugate is administered intravenously. In some embodiments, the anti-HER2 antibody-drug conjugate is ado-trastuzumab emtansine.

In some embodiments, the anti-HER2 antibody-drug conjugate is administered once every 21-day treatment cycle at a dose of about 4 mg/kg for the first dose of the anti-HER2 antibody-drug conjugate administered to the subject followed by subsequent doses of about 3.6 mg/kg, wherein the anti-HER2 antibody-drug conjugate is administered intravenously. In some embodiments, the anti-HER2 antibody-drug conjugate is administered once every 21-day treatment cycle at a dose of 5 mg/kg for the first dose of the anti-HER2 antibody-drug conjugate administered to the subject followed by subsequent doses of 3.6 mg/kg, wherein the anti-HER2 antibody-drug conjugate is administered intravenously. In some embodiments, the anti-HER2 antibody-drug conjugate is ado-trastuzumab emtansine.

In some embodiments, the dose of the trastuzumab deruxtecan during the first 21 day treatment cycle is 5.4 mg/kg and the dose of the trastuzumab deruxtecan during the subsequent 21 day treatment cycles is 4.4 mg/kg. In some embodiments, the dose of the trastuzumab deruxtecan during the first 21 day treatment cycle is 5.4 mg/kg and the dose of the trastuzumab deruxtecan during the subsequent 21 day treatment cycles is 3.2 mg/kg.

In some embodiments, the anti-HER2 antibody-drug conjugate is prepared and administered according to instructions in the package insert. In some embodiments, the anti-HER2 antibody-drug conjugate is administered intravenously or subcutaneously under the direction of medical personnel. In some embodiments, the anti-HER2 antibody-drug conjugate is stored according to the package insert. In some embodiments, the anti-HER2 antibody-drug conjugate is ado-trastuzumab emtansine. In some embodiments, the anti-HER2 antibody-drug conjugate is trastuzumab deruxtecan.

### F. Combination Therapy

Provided herein are methods of treatment comprising administering to the subject a combination therapy comprising tucatinib and an anti-HER2 antibody-drug conjugate. In some embodiments, the combination therapy consists essentially of tucatinib and an anti-HER2 antibody-drug conjugate. In some embodiments, the combination therapy consists of tucatinib and an anti-HER2 antibody-drug conjugate.

In some embodiments, the tucatinib and the anti-HER2 antibody-drug conjugate (e.g., trastuzumab deruxtecan or ado-trastuzumab emtansine) are administered to the subject on a 21 day treatment cycle. In some embodiments, the tucatinib is administered to the subject at a dose of about 150 mg to about 650 mg. In some embodiments, the tucatinib is administered to the subject twice per day. In some embodiments, the tucatinib is administered to the subject at a dose of about 300 mg twice per day. In some embodiments, tucatinib is administered to the subject at a dose of about 600 mg once per day. In some embodiments, tucatinib is administered to the subject twice per day on each day of a 21 day treatment cycle. In some embodiments, the tucatinib is administered to the subject orally.

In some embodiments, the anti-HER2 antibody-drug conjugate (e.g., trastuzumab deruxtecan or ado-trastuzumab emtansine) is administered at a dose of about 4 mg/kg once about every 3 weeks and the anti-HER2 antibody-drug conjugate (e.g., trastuzumab deruxtecan or ado-trastuzumab emtansine) is administered intravenously.

In some embodiments, ado-trastuzumab emtansine is administered at a dose of about 3.6 mg/kg once about every 3 weeks and the ado-trastuzumab emtansine is administered intravenously. In some embodiments, ado-trastuzumab emtansine is administered at a dose of about 3 mg/kg once about every 3 weeks and the ado-trastuzumab emtansine is administered intravenously. In some embodiments, ado-trastuzumab emtansine is administered at a dose of about 2.4 mg/kg once about every 3 weeks and ado-trastuzumab emtansine is administered intravenously.

In some embodiments, trastuzumab deruxtecan is administered at a dose of about 3.6 mg/kg to about 7 mg/kg every 3 weeks. In some embodiments, trastuzumab deruxtecan is administered at a dose of about 5.4 mg/kg once about every 3 weeks and the trastuzumab deruxtecan is administered intravenously. In some embodiments, trastuzumab deruxtecan is administered at a dose of about 4.4 mg/kg once about every 3 weeks and the trastuzumab deruxtecan is administered intravenously. In some embodiments, trastuzumab deruxtecan is administered at a dose of about 3.2 mg/kg once about every 3 weeks and the trastuzumab deruxtecan is administered intravenously. In some embodiments, the anti-HER2 antibody-drug conjugate is administered to the subject once per 21 day treatment cycle.

In some embodiments, the anti-HER2 antibody-drug conjugate is administered about once every 1 week, once about every 2 weeks, once about every 3 weeks, or once about every 4 weeks. In some embodiments, the anti-HER2 antibody-drug conjugate is administered once about every 3 weeks. In some embodiments, the anti-HER2 antibody-drug conjugate is trastuzumab deruxtecan and is administered about once every 1 week, once about every 2 weeks, once about every 3 weeks, or once about every 4 weeks. In some embodiments, the trastuzumab deruxtecan is administered once about every 3 weeks.

Provided herein are methods of treatment comprising administering to the subject a combination therapy comprising tucatinib and ado-trastuzumab emtansine or trastuzumab deruxtecan. In some embodiments, the combination therapy consists essentially of tucatinib and ado-trastuzumab emtansine or trastuzumab deruxtecan. In some embodiments, the combination therapy consists of tucatinib and ado-trastuzumab emtansine or trastuzumab deruxtecan.

In some embodiments, the tucatinib and ado-trastuzumab emtansine are administered to the subject on a 21 day treatment cycle. In some embodiments, tucatinib is administered to the subject at a dose of about 300 mg twice per day. In some embodiments, tucatinib is administered to the subject at a dose of 300 mg twice per day. In some embodiments, tucatinib is administered to the subject at a dose of about 600 mg once per day. In some embodiments, tucatinib is administered to the subject at a dose of 600 mg once per day. In some embodiments, tucatinib is administered to the subject twice per day on each day of a 21 day treatment cycle. In some embodiments, the tucatinib is administered to the subject orally. In some embodiments, ado-trastuzumab emtansine or trastuzumab deruxtecan is administered at a dose of about 3.6 mg/kg once about every 3 weeks and ado-trastuzumab emtansine or trastuzumab deruxtecan is administered intravenously. In some embodiments, ado-trastuzumab emtansine or trastuzumab deruxtecan is administered at a dose of about 4 mg/kg once about every 3 weeks and ado-trastuzumab emtansine or trastuzumab deruxtecan is administered intravenously.

### G. Treatment Outcome

In some embodiments, treating the subject comprises inhibiting breast cancer cell growth, inhibiting breast cancer cell proliferation, inhibiting breast cancer cell migration, inhibiting breast cancer cell invasion, decreasing or eliminating one or more signs or symptoms of breast cancer, reducing the size (e.g., volume) of a breast cancer tumor, reducing the number of breast cancer tumors, reducing the number of breast cancer cells, inducing breast cancer cell necrosis, pyroptosis, oncosis, apoptosis, autophagy, or other cell death, increasing survival time of the subject, or enhancing the therapeutic effects of another drug or therapy.

In some embodiments, treating the subject comprises inhibiting brain mestastasis cell growth, inhibiting brain mestastasis cell proliferation, inhibiting brain mestastasis cell migration, inhibiting brain mestastasis cell invasion, decreasing or eliminating one or more signs or symptoms of a brain mestastasis, reducing the size (e.g., volume) of a brain mestastasis, reducing the number of brain mestastasis, inducing brain mestastasis cell necrosis, pyroptosis, oncosis, apoptosis, autophagy, or other cell death, increasing survival time of the subject, or enhancing the therapeutic effects of another drug or therapy.

In some embodiments, treating the subject as described herein results in a tumor growth inhibition (TGI) index that is between about 10% and 70% (e.g., about 10%, 15%, 20%, 25%, 30%, 35%, 40%, 45%, 50%, 55%, 60%, 65%, or 70%). Preferably, treating the subject results in a TGI index that is at least about 70% (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%). More preferably, treating the subject results in a TGI index that is at least about 85% (e.g., about 85%, 86%, 87%, 88%, 89%, 90%, 91%, 92%, 93%, 94%, 95%, 96%, 97%, 98%, 99%, or 100%). Even more preferably, treating the subject results in a TGI index that is at least about 95% (e.g., about 95%, 96%, 97%, 98%, 99%, or 100%). Most preferably, treating the subject results in a TGI index that is about 100% or more (e.g., about 100%, 101%, 102%, 103%, 104%, 105%, 106%, 107%, 108%, 109%, 110%, 111%, 112%, 113%, 114%, 115%, 116%, 117%, 118%, 119%, 120%, 125%, 130%, 135%, 140%, 145%, 150%, or more).

In particular embodiments, treating the subject with tucatinib and an anti-HER2 antibody-drug conjugate results in a TGI index that is greater than the TGI index that is observed when tucatinib or the anti-HER2 antibody-drug conjugate is used alone. In some instances, treating the subject results in a TGI index that is greater than the TGI index that is observed when tucatinib is used alone. In other instances, treating the subject results in a TGI index that is greater than the TGI index that is observed when the anti-HER2 antibody-drug conjugate is used alone. In some embodiments, treating the subject results in a TGI index that is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the TGI index that is observed when tucatinib or the anti-HER2 antibody-drug conjugate is used alone. In some embodiments, the anti-HER2 antibody-drug conjugate is ado-trastuzumab emtansine. In some embodiments, the anti-HER2 antibody-drug conjugate is trastuzumab deruxtecan.

In some embodiments, the combination of the tucatinib and the anti-HER2 antibody-drug conjugate is synergistic. In particular embodiments, with respect to the synergistic combination, treating the subject results in a TGI index that is greater than the TGI index that would be expected if the combination of tucatinib and the anti-HER2 antibody-drug conjugate produced an additive effect. In some instances, the TGI index observed when a combination of tucatinib and an anti-HER2 antibody-drug conjugate is administered is at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, or 80% greater than the TGI index that would be expected if the combination of tucatinib and the anti-HER2 antibody-drug conjugate produced an additive effect. In some embodiments, the anti-HER2 antibody-drug conjugate is ado-trastuzumab emtansine. In some embodiments, the anti-HER2 antibody-drug conjugate is trastuzumab deruxtecan.

In one aspect, a method of treating cancer with tucatinib and an anti-HER2 antibody-drug conjugate (e.g., trastuzumab deruxtecan or ado-trastuzumab emtansine) as described herein results in an improvement in one or more therapeutic effects in the subject after administration of a combination of tucatinib and an anti-HER2 antibody-drug conjugate (e.g., trastuzumab deruxtecan or ado-trastuzumab emtansine) relative to a baseline. In some embodiments, the one or more therapeutic effects is the size of the tumor derived from the breast cancer, the objective response rate, the duration of response, the time to response, progression free survival, overall survival, or any combination thereof. In one embodiment, the one or more therapeutic effects is the size of the tumor derived from the breast cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment with a combination of tucatinib and an anti-HER2 antibody-drug conjugate (e.g., trastuzumab deruxtecan or ado-trastuzumab emtansine) as described herein may include the following criteria (RECIST Criteria 1.1):

| | Category | Criteria |
|---|---|---|
| Based on target | Complete Response | Disappearance of all target lesions. Any pathological |
| lesions | (CR) | lymph nodes must have reduction in short axis to <10 |
| | | mm. |
| | Partial Response | ≥30% decrease in the sum of the longest diameter (LD) |
| | (PR) | of target lesions, taking as reference the baseline sum of |
| | | LDs. |
| | Stable Disease (SD) | Neither sufficient shrinkage to qualify for PR nor |
| | | sufficient increase to qualify for PD, taking as reference |
| | | the smallest sum of LDs while in trial. |
| | Progressive | ≥20% (and ≥5 mm) increase in the sum of the LDs of |
| | Disease (PD) | target lesions, taking as reference the smallest sum of |
| | | the target LDs recorded while in trial or the appearance |
| | | of one or more new lesions. |
| Based on non- | CR | Disappearance of all non-target lesions and |
| target lesions | | normalization of tumor marker level. All lymph nodes |
| | | must be non-pathological in size (<10 mm short axis). |
| | SD | Persistence of one or more non-target lesion(s) or/and |
| | | maintenance of tumor marker level above the normal |
| | | limits. |
| | PD | Appearance of one or more new lesions and/or |
| | | unequivocal progression of existing non-target lesions. |

In one embodiment of the methods or uses or product for uses provided herein, the effectiveness of treatment with a combination of tucatinib and an anti-HER2 antibody-drug conjugate (e.g., trastuzumab deruxtecan or ado-trastuzumab emtansine) as described herein is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with a combination of tucatinib and an anti-HER2 antibody-drug conjugate (e.g., trastuzumab deruxtecan or ado-trastuzumab emtansine) described herein is assessed by measuring the size of a tumor derived from the cancer (e.g., breast cancer). In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the combination of tucatinib and the anti-HER2 antibody-drug conjugate (e.g., trastuzumab deruxtecan or ado-trastuzumab emtansine). In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the combination of tucatinib and the anti-HER2 antibody-drug conjugate (e.g., trastuzumab deruxtecan or ado-trastuzumab emtansine). In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In some embodiments, the size of a tumor derived from a breast cancer is measured by mammography, sonography or magnetic resonance imaging (MRI). See Gruber et. al., 2013, BMC Cancer. 13:328.

In one embodiment of the methods or uses or product for uses provided described herein, response to treatment with a combination of tucatinib and an anti-HER2 antibody-drug conjugate (e.g., trastuzumab deruxtecan or ado-trastuzumab emtansine) described herein, promotes regression of a tumor derived from the cancer (e.g., breast cancer). In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the tucatinib described herein. In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of tucatinib described herein. In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In some embodiments, regression of a tumor is determined by mammography, sonography or magnetic resonance imaging (MRI). See Gruber et. al., 2013, BMC Cancer. 13:328.

In one embodiment of the methods or uses or product for uses described herein, response to treatment with a combination of tucatinib and an anti-HER2 antibody-drug conjugate as described herein is assessed by measuring the duration of response to a combination of tucatinib and an anti-HER2 antibody-drug conjugate after administration of the combination of tucatinib and the anti-HER2 antibody-drug conjugate. In some embodiments, the duration of response to a combination of tucatinib and an anti-HER2 antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the combination of tucatinib and the anti-HER2 antibody-drug conjugate. In some embodiments, the duration of response to a combination of tucatinib and an anti-HER2 antibody-drug conjugate is at least about 6 months after administration of the combination of tucatinib and the anti-HER2 antibody-drug conjugate. In some embodiments, the duration of response to a combination of tucatinib and an anti-HER2 antibody-drug conjugate is at least about one year after administration of the combination of tucatinib and the anti-HER2 antibody-drug conjugate. In some embodiments, the duration of response to a combination of tucatinib and an anti-HER2 antibody-drug conjugate is at least about two years after administration of the combination of tucatinib and the anti-HER2 antibody-drug conjugate. In some embodiments, the duration of response to a combination of tucatinib and an anti-HER2 antibody-drug conjugate is at least about three years after administration of the combination of tucatinib and the anti-HER2 antibody-drug conjugate. In some embodiments, the duration of response to a combination of tucatinib and an anti-HER2 antibody-drug conjugate is at least about four years after administration of the combination of tucatinib and the anti-HER2 antibody-drug conjugate. In some embodiments, the duration of response to a combination of tucatinib and an anti-HER2 antibody-drug conjugate is at least about five years after administration of the combination of tucatinib and the anti-HER2 antibody-drug conjugate. In some embodiments, the duration of response to a combination of tucatinib and an anti-HER2 antibody-drug conjugate is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the combination of tucatinib and the anti-HER2 antibody-drug conjugate. In some embodiments, the duration of response to a combination of tucatinib and an anti-HER2 antibody-drug conjugate is at least 6 months after administration of the combination of tucatinib and the anti-HER2 antibody-drug conjugate. In some embodiments, the duration of response to a combination of tucatinib and an anti-HER2 antibody-drug conjugate is at least one year after administration of the combination of tucatinib and the anti-HER2 antibody-drug conjugate. In some embodiments, the duration of response to a combination of tucatinib and an anti-HER2 antibody-drug conjugate is at least two years after administration of the combination of tucatinib and the anti-HER2 antibody-drug conjugate. In some embodiments, the duration of response to a combination of tucatinib and an anti-HER2 antibody-drug conjugate is at least three years after administration of the combination of tucatinib and the anti-HER2 antibody-drug conjugate. In some embodiments, the duration of response to a combination of tucatinib and an anti-HER2 antibody-drug conjugate is at least four years after administration of the combination of tucatinib and the anti-HER2 antibody-drug conjugate. In some embodiments, the duration of response to a combination of tucatinib and an anti-HER2 antibody-drug conjugate is at least five years after administration of the combination of tucatinib and the anti-HER2 antibody-drug conjugate. In some embodiments, the anti-HER2 antibody-drug conjugate is ado-trastuzumab emtansine. In some embodiments, the anti-HER2 antibody-drug conjugate is ado-trastuzumab emtansine.

In one embodiment of the methods or uses or product for uses provided herein, response to treatment with a combination of tucatinib and an anti-HER2 antibody-drug conjugate (e.g., trastuzumab deruxtecan or ado-trastuzumab emtansine) described herein is assessed by measuring the size of a brain metastasis derived from the cancer (e.g., breast cancer). In one embodiment, the size of a brain metastasis derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the brain metastasis derived from the cancer before administration of the combination of tucatinib and the anti-HER2 antibody-drug conjugate (e.g., trastuzumab deruxtecan or ado-trastuzumab emtansine). In one embodiment, the size of a brain metastasis derived from the cancer is reduced by at least about 10%-80%. In one embodiment, the size of a brain metastasis derived from the cancer is reduced by at least about 20%-80%. In one embodiment, the size of a brain metastasis derived from the cancer is reduced by at least about 30%-80%. In one embodiment, the size of a brain metastasis derived from the cancer is reduced by at least about 40%-80%. In one embodiment, the size of a brain metastasis derived from the cancer is reduced by at least about 50%-80%. In one embodiment, the size of a brain metastasis derived from the cancer is reduced by at least about 60%-80%. In one embodiment, the size of a brain metastasis derived from the cancer is reduced by at least about 70%-80%. In one embodiment, the size of a brain metastasis derived from the cancer is reduced by at least about 80%. In one embodiment, the size of a brain metastasis derived from the cancer is reduced by at least about 85%. In one embodiment, the size of a brain metastasis derived from the cancer is reduced by at least about 90%. In one embodiment, the size of a brain metastasis derived from the cancer is reduced by at least about 95%. In one embodiment, the size of a brain metastasis derived from the cancer is reduced by at least about 98%. In one embodiment, the size of a brain metastasis derived from the cancer is reduced by at least about 99%. In one embodiment, the size of a brain metastasis derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the brain metastasis derived from the cancer before administration of the combination of tucatinib and the anti-HER2 antibody-drug conjugate (e.g., ado-trastuzumab emtansine). In one embodiment, the size of a brain metastasis derived from the cancer is reduced by at least 10%-80%. In one embodiment, the size of a brain metastasis derived from the cancer is reduced by at least 20%-80%. In one embodiment, the size of a brain metastasis derived from the cancer is reduced by at least 30%-80%. In one embodiment, the size of a brain metastasis derived from the cancer is reduced by at least 40%-80%. In one embodiment, the size of a brain metastasis derived from the cancer is reduced by at least 50%-80%. In one embodiment, the size of a brain metastasis derived from the cancer is reduced by at least 60%-80%. In one embodiment, the size of a brain metastasis derived from the cancer is reduced by at least 70%-80%. In one embodiment, the size of a brain metastasis derived from the cancer is reduced by at least 80%. In one embodiment, the size of a brain metastasis derived from the cancer is reduced by at least 85%. In one embodiment, the size of a brain metastasis derived from the cancer is reduced by at least 90%. In one embodiment, the size of a brain metastasis derived from the cancer is reduced by at least 95%. In one embodiment, the size of a brain metastasis derived from the cancer is reduced by at least 98%. In one embodiment, the size of a brain metastasis derived from the cancer is reduced by at least 99%. In one embodiment, the size of a brain metastasis derived from the cancer is reduced by 100%.

In one embodiment of the methods or uses or product for uses provided described herein, response to treatment with a combination of tucatinib and an anti-HER2 antibody-drug conjugate (e.g., trastuzumab deruxtecan or ado-trastuzumab emtansine) described herein, promotes regression of a brain metastasis derived from the cancer (e.g., breast cancer). In one embodiment, a brain metastasis derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the brain metastasis derived from the cancer before administration of the combination of tucatinib and the anti-HER2 antibody-drug conjugate (e.g., trastuzumab deruxtecan or ado-trastuzumab emtansine) described herein. In one embodiment, a brain metastasis derived from the cancer regresses by at least about 10% to about 80%. In one embodiment, a brain metastasis derived from the cancer regresses by at least about 20% to about 80%. In one embodiment, a brain metastasis derived from the cancer regresses by at least about 30% to about 80%. In one embodiment, a brain metastasis derived from the cancer regresses by at least about 40% to about 80%. In one embodiment, a brain metastasis derived from the cancer regresses by at least about 50% to about 80%. In one embodiment, a brain metastasis derived from the cancer regresses by at least about 60% to about 80%. In one embodiment, a brain metastasis derived from the cancer regresses by at least about 70% to about 80%. In one embodiment, a brain metastasis derived from the cancer regresses by at least about 80%. In one embodiment, a brain metastasis derived from the cancer regresses by at least about 85%. In one embodiment, a brain metastasis derived from the cancer regresses by at least about 90%. In one embodiment, a brain metastasis derived from the cancer regresses by at least about 95%. In one embodiment, a brain metastasis derived from the cancer regresses by at least about 98%. In one embodiment, a brain metastasis derived from the cancer regresses by at least about 99%. In one embodiment, a brain metastasis derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the brain metastasis derived from the cancer before administration of the combination of tucatinib and the anti-HER2 antibody-drug conjugate (e.g., trastuzumab deruxtecan or ado-trastuzumab emtansine) as described herein. In one embodiment, a brain metastasis derived from the cancer regresses by at least 10% to 80%. In one embodiment, a brain metastasis derived from the cancer regresses by at least 20% to 80%. In one embodiment, a brain metastasis derived from the cancer regresses by at least 30% to 80%. In one embodiment, a brain metastasis derived from the cancer regresses by at least 40% to 80%. In one embodiment, a brain metastasis derived from the cancer regresses by at least 50% to 80%. In one embodiment, a brain metastasis derived from the cancer regresses by at least 60% to 80%. In one embodiment, a brain metastasis derived from the cancer regresses by at least 70% to 80%. In one embodiment, a brain metastasis derived from the cancer regresses by at least 80%. In one embodiment, a brain metastasis derived from the cancer regresses by at least 85%. In one embodiment, a brain metastasis derived from the cancer regresses by at least 90%. In one embodiment, a brain metastasis derived from the cancer regresses by at least 95%. In one embodiment, a brain metastasis derived from the cancer regresses by at least 98%. In one embodiment, a brain metastasis derived from the cancer regresses by at least 99%. In one embodiment, a brain metastasis derived from the cancer regresses by 100%.

In some embodiments, the size, progression, regression, and/or response of a brain metastasis to administration of a combination of tucatinib and an anti-HER2 antibody-drug conjugate (e.g., trastuzumab deruxtecan or ado-trastuzumab emtansine) as described herein is determined using one or more of the RANO-BM criteria. See, for example, Lin, N. U. et al. The Lancet 16 (June 2015): e270-e278.

### H. Compositions

In another aspect, the present disclosure provides a pharmaceutical composition comprising tucatinib and a pharmaceutically acceptable carrier. In another aspect, the present disclosure provides a pharmaceutical composition comprising an anti-HER2 antibody-drug conjugate (e.g., trastuzumab deruxtecan or ado-trastuzumab emtansine) and a pharmaceutically acceptable carrier. In another aspect, the present disclosure provides a pharmaceutical composition comprising tucatinib, an anti-HER2 antibody-drug conjugate (e.g., trastuzumab deruxtecan or ado-trastuzumab emtansine), and a pharmaceutically acceptable carrier.

In some embodiments, tucatinib is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, tucatinib is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, tucatinib is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, tucatinib is present at a concentration at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM).

In some embodiments, the anti-HER2 antibody-drug conjugate is present at a concentration between about 0.1 nM and 10 nM (e.g., about 0.1, 0.2, 0.3, 0.4, 0.5 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 nM). In other embodiments, the anti-HER2 antibody-drug conjugate is present at a concentration between about 10 nM and 100 nM (e.g., about 10, 15, 20, 25, 30, 35, 40, 45, 50, 55, 60, 65, 70, 75, 80, 85, 90, 95, or 100 nM). In some other embodiments, the anti-HER2 antibody-drug conjugate is present at a concentration between about 100 nM and 1,000 nM (e.g., about 100, 150, 200, 250, 300, 350, 400, 450, 500, 550, 600, 650, 700, 750, 800, 850, 900, 950, or 1,000 nM). In yet other embodiments, the anti-HER2 antibody-drug conjugate is present at a concentration of at least about 1,000 nM to 10,000 nM (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, or more nM). In some embodiments, the anti-HER2 antibody-drug conjugate is ado-trastuzumab emtansine. In some embodiments, the anti-HER2 antibody-drug conjugate is trastuzumab deruxtecan.

Preparation of ado-trastuzumab emtasine, methods of using same, and additional examples of pharmaceutical compositions of ado-trastuzumab emtasine and methods of preparation thereof are described in, for example, U.S. Pat. Nos. 7,575,748; 7,097,840; and 8,337,856; which are incorporated by reference herein in their entireties.

The pharmaceutical compositions of the present disclosure may be prepared by any of the methods well-known in the art of pharmacy. Pharmaceutically acceptable carriers suitable for use with the present disclosure include any of the standard pharmaceutical carriers, buffers and excipients, including phosphate-buffered saline solution, water, and emulsions (such as an oil/water or water/oil emulsion), and various types of wetting agents or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent.

The pharmaceutical compositions of the present disclosure can include a combination of drugs (e.g., tucatinib and an anti-HER2 antibody-drug conjugate (e.g., trastuzumab deruxtecan or ado-trastuzumab emtansine)), or any pharmaceutically acceptable salts thereof, as active ingredients and a pharmaceutically acceptable carrier or excipient or diluent. A pharmaceutical composition may optionally contain other therapeutic ingredients.

The compositions (e.g., comprising tucatinib and an anti-HER2 antibody-drug conjugate (e.g., trastuzumab deruxtecan or ado-trastuzumab emtansine)) can be combined as the active ingredients in intimate admixture with a suitable pharmaceutical carrier or excipient according to conventional pharmaceutical compounding techniques. Any carrier or excipient suitable for the form of preparation desired for administration is contemplated for use with the compounds disclosed herein.

The pharmaceutical compositions include those suitable for oral, topical, parenteral, pulmonary, nasal, or rectal administration. The most suitable route of administration in any given case will depend in part on the nature and severity of the cancer condition and also optionally the HER2 status or stage of the cancer.

Other pharmaceutical compositions include those suitable for systemic (e.g., enteral or parenteral) administration. Systemic administration includes oral, rectal, sublingual, or sublabial administration. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In particular embodiments, pharmaceutical compositions of the present disclosure may be administered intratumorally.

Compositions for pulmonary administration include, but are not limited to, dry powder compositions consisting of the powder of a compound described herein (e.g., tucatinib and an anti-HER2 antibody-drug conjugate (e.g., trastuzumab deruxtecan or ado-trastuzumab emtansine)), or a salt thereof, and the powder of a suitable carrier or lubricant. The compositions for pulmonary administration can be inhaled from any suitable dry powder inhaler device known to a person skilled in the art.

Compositions for systemic administration include, but are not limited to, dry powder compositions consisting of the composition as set forth herein (e.g., tucatinib and an anti-HER2 antibody-drug conjugate (e.g., trastuzumab deruxtecan or ado-trastuzumab emtansine)) and the powder of a suitable carrier or excipient. The compositions for systemic administration can be represented by, but not limited to, tablets, capsules, pills, syrups, solutions, and suspensions.

In some embodiments, the compositions (e.g., tucatinib and an anti-HER2 antibody-drug conjugate (e.g., trastuzumab deruxtecan or ado-trastuzumab emtansine)) further include a pharmaceutical surfactant. In other embodiments, the compositions further include a cryoprotectant. In some embodiments, the cryoprotectant is selected from the group consisting of glucose, sucrose, trehalose, lactose, sodium glutamate, PVP, HPβCD, CD, glycerol, maltose, mannitol, and saccharose.

Pharmaceutical compositions or medicaments for use in the present disclosure can be formulated by standard techniques using one or more physiologically acceptable carriers or excipients. Suitable pharmaceutical carriers are described herein and in Remington: The Science and Practice of Pharmacy, 21st Ed., University of the Sciences in Philadelphia, Lippencott Williams & Wilkins (2005).

Controlled-release parenteral formulations of the compositions (e.g., tucatinib and an anti-HER2 antibody-drug conjugate (e.g., trastuzumab deruxtecan or ado-trastuzumab emtansine)) can be made as implants, oily injections, or as particulate systems. For a broad overview of delivery systems see Banga, A. J., THERAPEUTIC PEPTIDES AND PROTEINS: FORMULATION, PROCESSING, AND DELIVERY SYSTEMS, Technomic Publishing Company, Inc., Lancaster, Pa., (1995), which is incorporated herein by reference. Particulate systems include microspheres, microparticles, microcapsules, nanocapsules, nanospheres, and nanoparticles.

Polymers can be used for ion-controlled release of compositions of the present disclosure. Various degradable and nondegradable polymeric matrices for use in controlled drug delivery are known in the art (Langer R., Accounts Chem. Res., 26:537-542 (1993)). For example, the block copolymer, polaxamer 407 exists as a viscous yet mobile liquid at low temperatures but forms a semisolid gel at body temperature. It has been shown to be an effective vehicle for formulation and sustained delivery of recombinant interleukin 2 and urease (Johnston et al., Pharm. Res., 9:425-434 (1992); and Pec et al., J. Parent. Sci. Tech., 44(2):58 65 (1990)). Alternatively, hydroxyapatite has been used as a microcarrier for controlled release of proteins (Ijntema et al., Int. J. Pharm., 112:215-224 (1994)). In yet another aspect, liposomes are used for controlled release as well as drug targeting of the lipid-capsulated drug (Betageri et al., LIPOSOME DRUG DELIVERY SYSTEMS, Technomic Publishing Co., Inc., Lancaster, Pa. (1993)). Numerous additional systems for controlled delivery of therapeutic proteins are known. See, e.g., U.S. Pat. Nos. 5,055,303, 5,188,837, 4,235,871, 4,501,728, 4,837,028 4,957,735 and 5,019,369, 5,055,303; 5,514,670; 5,413,797; 5,268,164; 5,004,697; 4,902,505; 5,506,206, 5,271,961; 5,254,342 and 5,534,496, each of which is incorporated herein by reference.

For oral administration of a combination of tucatinib and an anti-HER2 antibody-drug conjugate (e.g., trastuzumab deruxtecan or ado-trastuzumab emtansine), a pharmaceutical composition or a medicament can take the form of, for example, a tablet or a capsule prepared by conventional means with a pharmaceutically acceptable excipient. The present disclosure provides tablets and gelatin capsules comprising tucatinib and an anti-HER2 antibody-drug conjugate (e.g., trastuzumab deruxtecan or ado-trastuzumab emtansine), or a dried solid powder of these drugs, together with (a) diluents or fillers, e.g., lactose, dextrose, sucrose, mannitol, sorbitol, cellulose (e.g., ethyl cellulose, microcrystalline cellulose), glycine, pectin, polyacrylates or calcium hydrogen phosphate, calcium sulfate, (b) lubricants, e.g., silica, talcum, stearic acid, magnesium or calcium salt, metallic stearates, colloidal silicon dioxide, hydrogenated vegetable oil, corn starch, sodium benzoate, sodium acetate or polyethyleneglycol; for tablets also (c) binders, e.g., magnesium aluminum silicate, starch paste, gelatin, tragacanth, methylcellulose, sodium carboxymethylcellulose, polyvinylpyrrolidone or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, or sweetening agents as appropriate. If desired, preparations for oral administration can be suitably formulated to give controlled release of the active compound(s).

Typical formulations for topical administration of tucatinib and an anti-HER2 antibody-drug conjugate (e.g., trastuzumab deruxtecan or ado-trastuzumab emtansine) include creams, ointments, sprays, lotions, and patches. The pharmaceutical composition can, however, be formulated for any type of administration, e.g., intradermal, subdermal, intravenous, intramuscular, subcutaneous, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral or rectal administration is also contemplated.

Suitable formulations for transdermal application include an effective amount of one or more compounds described herein, optionally with a carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

The compositions and formulations set forth herein (e.g., tucatinib and an anti-HER2 antibody-drug conjugate (e.g., trastuzumab deruxtecan or ado-trastuzumab emtansine)) can be formulated for parenteral administration by injection, for example by bolus injection or continuous infusion. Formulations for injection can be presented in unit dosage form, for example, in ampules or in multi-dose containers, with an added preservative. Injectable compositions are preferably aqueous isotonic solutions or suspensions, and suppositories are preferably prepared from fatty emulsions or suspensions. The compositions may be sterilized or contain adjuvants, such as preserving, stabilizing, wetting or emulsifying agents, solution promoters, salts for regulating the osmotic pressure or buffers. Alternatively, the active ingredient(s) can be in powder form for constitution with a suitable vehicle, for example, sterile pyrogen-free water, before use. In addition, they may also contain other therapeutically valuable substances. The compositions are prepared according to conventional mixing, granulating or coating methods, respectively.

For administration by inhalation, the compositions (e.g., comprising tucatinib and an anti-HER2 antibody-drug conjugate (e.g., trastuzumab deruxtecan or ado-trastuzumab emtansine)) may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compositions (e.g., comprising tucatinib and an anti-HER2 antibody-drug conjugate (e.g., trastuzumab deruxtecan or ado-trastuzumab emtansine)) can also be formulated in rectal compositions, for example, suppositories or retention enemas, for example, containing conventional suppository bases, for example, cocoa butter or other glycerides.

Furthermore, the active ingredient(s) can be formulated as a depot preparation. Such long-acting formulations can be administered by implantation (for example, subcutaneously or intramuscularly) or by intramuscular injection. Thus, for example, one or more of the compounds described herein can be formulated with suitable polymeric or hydrophobic materials (for example as an emulsion in an acceptable oil) or ion exchange resins, or as sparingly soluble derivatives, for example, as a sparingly soluble salt.

### Pharmaceutical Compositions of Tucatinib

In some embodiments, a pharmaceutical composition comprising tucatinib and a pharmaceutically acceptable carrier is provided herein, wherein the pharmaceutical composition comprises a solid dispersion of tucatinib.

The solid dispersions are generally prepared by dissolving the drug substance and the dispersion polymer in a suitable solvent to form a feed solution, and then the feed solution may be spray dried to form the solid dispersion (and remove the solvent). Spray drying is a known process. Spray drying is generally performed by dissolving tucatinib and the dispersion polymer in a suitable solvent to prepare a feed solution. The feed solution may be pumped through an atomizer into a drying chamber. The feed solution can be atomized by conventional means known in the art, such as a two-fluid sonicating nozzle, a pressure nozzle, a rotating nozzle and a two-fluid non-sonicating nozzle. Then, the solvent is removed in the drying chamber to form the solid dispersion. A typical drying chamber uses hot gases, such as forced air, nitrogen, nitrogen-enriched air, or argon to dry particles. The size of the drying chamber may be adjusted to achieve particle properties or throughput. Although the solid dispersion are preferably prepared by conventional spray drying techniques, other techniques known in the art may be used, such as melt extrusion, freeze drying, rotary evaporation, drum drying or other solvent removal processes.

In some embodiments, a process of preparing a solid dispersion is provided, comprising: (a) dissolving tucatinib and a dispersion polymer in a suitable solvent; and (b) evaporating the solvent to form the solid dispersion. In certain embodiments, the evaporation of the solvent in step (b) is performed by spray drying, melt extrusion, freeze drying, rotary evaporation, drum drying or other solvent removal processes.

In certain embodiments, the dispersion polymer is selected from PVP-VA, methylacrylic acid methyl methacrylate copolymer, HPMCP, CAP, HPMCAS and HPMC and mixtures thereof. In certain embodiments, the dispersion polymer is selected from PVP-VA, methylacrylic acid methyl methacrylate copolymer, HPMCP, CAP, HPMCAS and HPMC. In certain embodiments, the dispersion polymer is selected from PVP-VA, Eudragit® L100, HPMCP H-55, CAP, HPMCAS Grade M, HPMC and mixtures thereof. In certain embodiments, the dispersion polymer is selected from PVP-VA, Eudragit® L100, HPMCP H-55, CAP, HPMCAS Grade M and HPMC.

In certain embodiments, the dispersion polymer is selected from PVP-VA, methylacrylic acid methyl methacrylate copolymer, HPMCP, CAP and HPMCAS, and mixtures thereof. In certain embodiments, the dispersion polymer is selected from PVP-VA, methylacrylic acid methyl methacrylate copolymer, HPMCP, CAP and HPMCAS. In certain embodiments, the dispersion polymer is selected from PVP-VA, Eudragit® L100, HPMCP H-55, CAP and HPMCAS Grade M, and mixtures thereof. In certain embodiments, the dispersion polymer is selected from PVP-VA, Eudragit® L100, HPMCP H-55, CAP and HPMCAS Grade M.

In certain embodiments, the dispersion polymer is selected from PVP-VA, methylacrylic acid methyl methacrylate copolymer, HPMCP, CAP and HPMC, and mixtures thereof. In certain embodiments, the dispersion polymer is selected from PVP-VA, methylacrylic acid methyl methacrylate copolymer, HPMCP, CAP and HPMC. In certain embodiments, the dispersion polymer is selected from PVP-VA, Eudragit® L100, HPMCP H-55, CAP and HPMC, and mixtures thereof. In certain embodiments, the dispersion polymer is selected from PVP-VA, Eudragit® L100, HPMCP H-55, CAP and HPMC

In certain embodiments, the dispersion polymer is selected from PVP-VA, methylacrylic acid methyl methacrylate copolymer, HPMCP and CAP, and mixtures thereof. In certain embodiments, the dispersion polymer is selected from PVP-VA, methylacrylic acid methyl methacrylate copolymer, HPMCP and CAP. In certain embodiments, the dispersion polymer is selected from PVP-VA, Eudragit® L100, HPMCP H-55 and CAP, and mixtures thereof. In certain embodiments, the dispersion polymer is selected from PVP-VA, Eudragit® L100, HPMCP H-55 and CAP. In certain embodiments, the dispersion polymer is PVP-VA.

In certain embodiments, the dispersion polymer is methylacrylic acid methyl methacrylate copolymer. In certain embodiments, the dispersion polymer is Eudragit®. In certain embodiments, the dispersion polymer is Eudragit® L100.

In certain embodiments, the dispersion polymer is HPMCP. In certain embodiments, the dispersion polymer is HPMCP H-55.

In certain embodiments, the dispersion polymer is CAP.

In certain embodiments, the dispersion polymer is HPMCAS. In certain embodiments, the dispersion polymer is HPMCAS Grade M.

In certain embodiments, the dispersion polymer is preferably neutral or basic.

In certain embodiments, the dispersion polymer is selected from PVP-VA and HPMC. In certain embodiments, the dispersion polymer is HPMC.

Suitable solvents are a solvent or mixture of solvents in which both tucatinib and the dispersion polymer have adequate solubility (solubility greater than 1 mg/mL). A mixture of solvents may be used if each component of the solid dispersion (i.e., tucatinib and dispersion polymer) require different solvents to obtain the desired solubility. The solvent may be volatile with a boiling point of 150° C. or less. In addition, the solvent should have relatively low toxicity and be removed from the dispersion to a level that is acceptable to The International Committee on Harmonization (“ICH”) guidelines. Removal of solvent to this level may require a subsequent processing step, such as tray drying. Examples of suitable solvents include, but are not limited to, alcohols, such as methanol (“MeOH”), ethanol (“EtOH”), n-propanol, isopropanol (“IPA”) and butanol; ketones, such as acetone, methyl ethyl ketone (“MEK”) and methyl isobutyl ketone; esters, such as ethyl acetate (“EA”) and propyl acetate; and various other solvents, such as tetrahydrofuran (“THF”), acetonitrile (“ACN”), methylene chloride, toluene and 1,1,1-trichloroethane. Lower volatility solvents, such as dimethyl acetate or dimethylsulfoxide (“DMSO”), may be used. Mixtures of solvents with water may also be used, so long as the polymer and tucatinib are sufficiently soluble to make the spray drying process practicable. Generally, due to the hydrophobic nature of low solubility drugs, non-aqueous solvents may be used, meaning the solvent comprises less than about 10 weight % water.

In certain embodiments, the suitable solvent is selected from MeOH and THF, and mixtures thereof. In certain embodiments, the suitable solvent is MeOH:THF solvent system of about 1:3. In certain embodiments, the suitable solvent is a 1:3 MeOH:THF solvent system.

In certain embodiments, the suitable solvent is selected from MeOH, THF and water, and mixtures thereof. In certain embodiments, the suitable solvent is selected from MeOH, THF and water. In certain embodiments, the suitable solvent is a THF:MeOH:water solvent system of about 80:10:10. In certain embodiments, the suitable solvent is a 80:10:10 THF:MeOH:water solvent system. In certain embodiments, the suitable solvent is a THF:MeOH:water solvent system of about 82:8:10. In certain embodiments, the suitable solvent is a 82:8:10 THF:MeOH:water solvent system. In certain embodiments, the suitable solvent is a THF:MeOH:water solvent system of about 82.2:8.2:9.6. In certain embodiments, the suitable solvent is a 82.2:8.2:9.6 THF:MeOH:water solvent system.

In certain embodiments, the amount of tucatinib in the solid dispersion ranges from about 0.1% to about 70% by weight relative to the dispersion polymer. In certain embodiments, the amount of tucatinib in the solid dispersion ranges from 0.1% to 70% by weight relative to the dispersion polymer.

In certain embodiments, the amount of tucatinib in the solid dispersion ranges from about 1% to about 60% by weight relative to the dispersion polymer. In certain embodiments, the amount of tucatinib in the solid dispersion ranges from 1% to 60% by weight relative to the dispersion polymer.

In certain embodiments, the amount of tucatinib in the solid dispersion ranges from about 5% to about 60% by weight relative to the dispersion polymer. In certain embodiments, the amount of tucatinib in the solid dispersion ranges from 5% to 60% by weight relative to the dispersion polymer.

In certain embodiments, the amount of tucatinib in the solid dispersion ranges from about 55% to about 65% by weight relative to the dispersion polymer. In certain embodiments, the amount of tucatinib in the solid dispersion ranges from 55% to 65% by weight relative to the dispersion polymer. In certain embodiments, the amount of tucatinib in the solid dispersion is about 60% by weight relative to the dispersion polymer. In certain embodiments, the amount of tucatinib in the solid dispersion is 60% by weight relative to the dispersion polymer.

In certain embodiments, the amount of tucatinib in the solid dispersion ranges from about 25% to about 35% by weight relative to the dispersion polymer. In certain embodiments, the amount of tucatinib in the solid dispersion ranges from 25% to 35% by weight relative to the dispersion polymer. In certain embodiments, the amount of tucatinib in the solid dispersion is about 30% by weight relative to the dispersion polymer. In certain embodiments, the amount of tucatinib in the solid dispersion is 30% by weight relative to the dispersion polymer.

In certain embodiments, the amount of tucatinib in the solid dispersion ranges from about 45% to about 55% by weight relative to the dispersion polymer. In certain embodiments, the amount of tucatinib in the solid dispersion ranges from 45% to 55% by weight relative to the dispersion polymer. In certain embodiments, the amount of tucatinib in the solid dispersion is about 50% by weight relative to the dispersion polymer. In certain embodiments, the amount of tucatinib in the solid dispersion is 50% by weight relative to the dispersion polymer.

In certain embodiments, the solid dispersion is an amorphous solid dispersion.

Another embodiment provides a pharmaceutical composition comprising a solid dispersion of tucatinib and a dispersion polymer, and a carrier or excipient.

Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005.

The pharmaceutical compositions may also include one or more additional components, such as buffers, dispersion agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug, i.e., a compound described herein or pharmaceutical composition thereof, or aid in the manufacturing of the pharmaceutical product, i.e., medicament (see Ansel; Gennaro; and Rowe above). The components of the pharmaceutical composition should be pharmaceutically acceptable.

Certain embodiments provide a pharmaceutical composition comprising: (a) about 1 to about 70 weight % of a solid dispersion of tucatinib; (b) about 0.1 to about 20 weight % of a disintegrant; (c) about 0.1 to about 25 weight % of an osmogen; (d) about 0.1 to about 10 weight % of a glidant; (e) about 0.1 to about 10 weight % of a lubricant; and (f) about 0.1 to about 25 weight % of a binder/diluent.

In certain embodiments, the pharmaceutical composition comprises: (a) 1 to 70 weight % of a solid dispersion of tucatinib; (b) 0.1 to 20 weight % of a disintegrant; (c) 0.1 to 25 weight % of an osmogen; (d) 0.1 to 10 weight % of a glidant; (e) 0.1 to 10 weight % of a lubricant; and (f) 0.1 to 25 weight % of a binder/diluent.

Certain embodiments provide a pharmaceutical composition comprising: (a) about 25 to about 60 weight % of a solid dispersion of tucatinib; (b) about 5 to about 15 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a binder/diluent.

In certain embodiments, the pharmaceutical composition comprises: (a) 25 to 60 weight % of a solid dispersion of tucatinib; (b) 5 to 15 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a binder/diluent.

Certain embodiments provide a pharmaceutical composition comprising: (a) about 40 to about 60 weight % of a solid dispersion of tucatinib; (b) about 5 to about 15 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a binder/diluent.

In certain embodiments, the pharmaceutical composition comprises: (a) 40 to 60 weight % of a solid dispersion of tucatinib; (b) 5 to 15 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a binder/diluent.

Certain embodiments provide a pharmaceutical composition comprising: (a) about 1 to about 70 weight % of a solid dispersion of tucatinib; (b) about 0.1 to about 20 weight % of a disintegrant; (c) about 0.1 to about 25 weight % of an osmogen; (d) about 0.1 to about 10 weight % of a glidant; (e) about 0.1 to about 10 weight % of a lubricant; and (f) about 0.1 to about 25 weight % of a filler.

In certain embodiments, the pharmaceutical composition comprises: (a) 1 to 70 weight % of a solid dispersion of tucatinib; (b) 0.1 to 20 weight % of a disintegrant; (c) 0.1 to 25 weight % of an osmogen; (d) 0.1 to 10 weight % of a glidant; (e) 0.1 to 10 weight % of a lubricant; and (f) 0.1 to 25 weight % of a filler.

Certain embodiments provide a pharmaceutical composition comprising: (a) about 25 to about 60 weight % of a solid dispersion of tucatinib; (b) about 1 to about 10 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a filler.

In certain embodiments, the pharmaceutical composition comprises: (a) 25 to 60 weight % of a solid dispersion of tucatinib; (b) 1 to 10 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a filler.

Certain embodiments provide a pharmaceutical composition comprising: (a) about 40 to about 60 weight % of a solid dispersion of tucatinib; (b) about 1 to about 10 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a filler.

In certain embodiments, the pharmaceutical composition comprises: (a) 40 to 60 weight % of a solid dispersion of tucatinib; (b) 1 to 10 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a filler.

In certain embodiments, the osmogen is selected from NaCl and KCl, and mixtures thereof.

In certain embodiments, the lubricant is magnesium stearate.

In certain embodiments, the glidant is colloidal silicon dioxide.

In certain embodiments, the binder/diluent is microcrystalline cellulose. In certain embodiments, the binder/diluent acts as both a binder and a diluent.

In certain embodiments, the binder is microcrystalline cellulose.

In certain embodiments, the diluent is microcrystalline cellulose.

In certain embodiments, the filler is lactose.

In certain embodiments, the disintegrant is selected from crospovidone and sodium bicarbonate (NaHCO₃), and mixtures thereof. In certain embodiments, the disintegrant is selected from crospovidone and sodium bicarbonate. In certain embodiments, the disintegrant is sodium bicarbonate. In certain embodiments, the disintegrant is crospovidone.

In certain embodiments, the composition contains sodium bicarbonate. tucatinib may slowly degrade, through hydrolysis or other means, to a carbamate impurity:

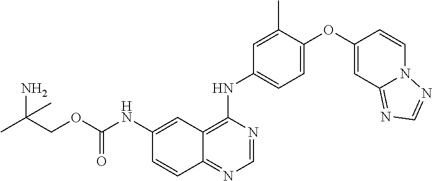 [SMILES: Cc1cc(Nc2ncnc3ccc(NC(=O)OCC(C)(C)N)cc23)ccc1Oc1ccn2ncnc2c1]

Sodium bicarbonate helps to slow the degradation to the carbamate impurity. Sodium bicarbonate also helps to provide consistent tablet disintegration when the tablets are exposed to different humidities.

Certain embodiments provide a pharmaceutical composition comprising: (a) tucatinib; and (b) sodium bicarbonate.

Certain embodiments provide a pharmaceutical composition comprising: (a) about 1 to about 70 weight % of a solid dispersion of tucatinib; and (b) about 0.1 to about 30 weight % sodium bicarbonate.

In certain embodiments, the pharmaceutical composition comprises: (a) 1 to 70 weight % of a solid dispersion of tucatinib; and (b) 0.1 to 30 weight % sodium bicarbonate.

Certain embodiments provide a pharmaceutical composition comprising: (a) about 1 to about 70 weight % of a solid dispersion of tucatinib; (b) about 0.1 to about 30 weight % sodium bicarbonate; and (c) the remaining weight is other pharmaceutically acceptable excipients and carriers.

In certain embodiments, the pharmaceutical composition comprises: (a) 1 to 70 weight % of a solid dispersion of tucatinib; (b) 0.1 to 30 weight % sodium bicarbonate; and (c) the remaining weight is other pharmaceutically acceptable excipients and carriers.

Certain embodiments provide a pharmaceutical composition comprising: (a) about 25 to about 60 weight % of a solid dispersion of tucatinib; and (b) about 1 to about 15 weight % of sodium bicarbonate.

In certain embodiments, the pharmaceutical composition comprises: (a) 25 to 60 weight % of a solid dispersion of tucatinib; and (b) 1 to 15 weight % of sodium bicarbonate.

Certain embodiments provide a pharmaceutical composition comprising: (a) about 25 to about 60 weight % of a solid dispersion of tucatinib; (b) about 1 to about 15 weight % of sodium bicarbonate; and (c) the remaining weight is other pharmaceutically acceptable excipients and carriers.

In certain embodiments, the pharmaceutical composition comprises: (a) 25 to 60 weight % of a solid dispersion of tucatinib; (b) 1 to 15 weight % of sodium bicarbonate; and (c) the remaining weight is other pharmaceutically acceptable excipients and carriers.

Certain embodiments provide a pharmaceutical composition comprising: (a) about 40 to about 60 weight % of a solid dispersion of tucatinib; and (b) about 1 to about 15 weight % of sodium bicarbonate.

In certain embodiments, the pharmaceutical composition comprises: (a) 40 to 60 weight % of a solid dispersion of tucatinib; and (b) 1 to 15 weight % of sodium bicarbonate.

Certain embodiments provide a pharmaceutical composition comprising: (a) about 40 to about 60 weight % of a solid dispersion of tucatinib; (b) about 1 to about 15 weight % of sodium bicarbonate; (c) the remaining weight is other pharmaceutically acceptable excipients and carriers.

In certain embodiments, the pharmaceutical composition comprises: (a) 40 to 60 weight % of a solid dispersion of tucatinib; (b) 1 to 15 weight % of sodium bicarbonate; (c) the remaining weight is other pharmaceutically acceptable excipients and carriers.

Certain embodiments provide a pharmaceutical composition comprising: (a) about 40 to about 60 weight % of a solid dispersion of tucatinib; (b) about 5 to about 15 weight % of a disintegrant which is selected from the group of crospovidone, sodium bicarbonate (NaHCO₃), and mixtures thereof; (c) about 15 to about 25 weight % of an osmogen which is selected from the group consisting of NaCl, KCl, and mixtures thereof; (d) about 0.1 to about 3 weight % of a glidant which is colloidal silicon dioxide; (e) about 0.1 to about 3 weight % of a lubricant which is magnesium stearate; and (f) about 10 to about 25 weight % of a binder/diluent which is microcrystalline cellulose.

In certain embodiments, the pharmaceutical composition comprises: (a) 40 to 60 weight % of a solid dispersion of tucatinib; (b) 5 to 15 weight % of a disintegrant which is selected from the group of crospovidone, sodium bicarbonate (NaHCO₃), and mixtures thereof; (c) 15 to 25 weight % of an osmogen which is selected from the group consisting of NaCl, KCl, and mixtures thereof; (d) 0.1 to 3 weight % of a glidant which is colloidal silicon dioxide; (e) 0.1 to 3 weight % of a lubricant which is magnesium stearate; and (f) 10 to 25 weight % of a binder/diluent which is microcrystalline cellulose.

Certain embodiments provide a pharmaceutical composition comprising: (a) about 40 to about 60 weight % of a solid dispersion of tucatinib; (b) about 1 to about 10 weight % of a disintegrant which is selected from the group of crospovidone, sodium bicarbonate (NaHCO₃), and mixtures thereof; (c) about 15 to about 25 weight % of an osmogen which is selected from the group consisting of NaCl, KCl, and mixtures thereof; (d) about 0.1 to about 3 weight % of a glidant which is colloidal silicon dioxide; (e) about 0.1 to about 3 weight % of a lubricant which is magnesium stearate; and (f) about 10 to about 25 weight % of a filler which is lactose.

In certain embodiments, the pharmaceutical composition comprises: (a) 40 to 60 weight % of a solid dispersion of tucatinib; (b) 1 to 10 weight % of a disintegrant which is selected from the group of crospovidone, sodium bicarbonate (NaHCO₃), and mixtures thereof; (c) 15 to 25 weight % of an osmogen n osmogen which is selected from the group consisting of NaCl, KCl, and mixtures thereof; (d) 0.1 to 3 weight % of a glidant which is colloidal silicon dioxide; (e) 0.1 to 3 weight % of a lubricant which is magnesium stearate; and (f) 10 to 25 weight % of a filler which is lactose.

In certain embodiments, the pharmaceutical composition is selected from the group consisting of:

| Function | Ingredient | % of Blend |
|---|---|---|
| API | Solid dispersion of tucatinib | about 50 |
| Disintegrant | Crospovidone - | about 6 |
| | Polyplasdone ® | |
| Osmogen | NaCl | about 5 |
| Osmogen | KCl | about 5 |
| Glidant | Colloidal Silicon Dioxide | about 0.5 |
| Lubricant | Magnesium Stearate | about 0.25 |

Extragranular

| | | |
|---|---|---|
| Binder/Diluent | Microcrystalline | about 19.25 |
| | cellulose - Avicel ® | |
| Osmogen | NaCl | about 4.625 |
| Osmogen | KCl | about 4.625 |
| Disintegrant | Polyplasdone | about 4 |
| Glidant | Colloidal Silicon Dioxide | about 0.5 |
| Lubricant | Magnesium Stearate | about 0.25 |

| Function | Ingredient | % of Blend |
|---|---|---|
| API | Solid dispersion of tucatinib | about 50 |
| Disintegrant | Crospovidone - | about 6 |
| | Polyplasdone ® | |
| Disintegrant | NaHCO₃ | about 3 |
| Osmogen | NaCl | about 5 |
| Osmogen | KCl | about 5 |
| Glidant | Colloidal Silicon Dioxide | about 0.5 |
| Lubricant | Magnesium Stearate | about 0.25 |

Extragranular

| | | |
|---|---|---|
| Binder/Diluent | Microcrystalline | about 16.25 |
| | cellulose - Avicel ® | |
| Osmogen | NaCl | about 4.625 |
| Osmogen | KCl | about 4.625 |
| Disintegrant | Polyplasdone | about 4 |
| Glidant | Colloidal Silicon Dioxide | about 0.5 |
| Lubricant | Magnesium Stearate | about 0.25 |

| Function | Ingredient | % of Blend |
|---|---|---|
| API | Solid dispersion of tucatinib | about 50 |
| Disintegrant | Crospovidone - | about 6 |
| | Polyplasdone ® | |
| Osmogen | NaCl | about 10.625 |
| Osmogen | KCl | about 10.625 |
| Filler | Lactose | about 21.25 |
| Glidant | Colloidal Silicon Dioxide | about 0.5 |
| Lubricant | Magnesium Stearate | aabout 0.25 |

Extragranular

| | | |
|---|---|---|
| Glidant | Colloidal Silicon Dioxide | about 0.5 |
| Lubricant | Magnesium Stearate | about 0.25 |

In certain embodiments, the pharmaceutical composition is selected from the group consisting of:

| Function | Ingredient | % of Blend |
|---|---|---|
| API | Solid dispersion of tucatinib | 50 |
| Disintegrant | Crospovidone - | 6 |
| | Polyplasdone ® | |
| Osmogen | NaCl | 5 |
| Osmogen | KCl | 5 |
| Glidant | Colloidal Silicon Dioxide | 0.5 |
| Lubricant | Magnesium Stearate | 0.25 |

Extragranular

| | | |
|---|---|---|
| Binder/Diluent | Microcrystalline | 19.25 |
| | cellulose - Avicel ® | |
| Osmogen | NaCl | 4.625 |
| Osmogen | KCl | 4.625 |
| Disintegrant | Polyplasdone | 4 |
| Glidant | Colloidal Silicon Dioxide | 0.5 |
| Lubricant | Magnesium Stearate | 0.25 |

| Function | Ingredient | % of Blend |
|---|---|---|
| API | Solid dispersion of tucatinib | 50 |
| Disintegrant | Crospovidone - | 6 |
| | Polyplasdone ® | |
| Disintegrant | NaHCO₃ | 3 |
| Osmogen | NaCl | 5 |
| Osmogen | KCl | 5 |
| Glidant | Colloidal Silicon Dioxide | 0.5 |
| Lubricant | Magnesium Stearate | 0.25 |

Extragranular

| | | |
|---|---|---|
| Binder/Diluent | Microcrystalline | 16.25 |
| | cellulose - Avicel ® | |
| Osmogen | NaCl | 4.625 |
| Osmogen | KCl | 4.625 |
| Disintegrant | Polyplasdone | 4 |
| Glidant | Colloidal Silicon Dioxide | 0.5 |
| Lubricant | Magnesium Stearate | 0.25 |

| Function | Ingredient | % of Blend |
|---|---|---|
| API | Solid dispersion of tucatinib | 50 |
| Disintegrant | Crospovidone - | 6 |
| | Polyplasdone ® | |
| Osmogen | NaCl | 10.625 |
| Osmogen | KCl | 10.625 |
| Filler | Lactose | 21.25 |
| Glidant | Colloidal Silicon Dioxide | 0.5 |
| Lubricant | Magnesium Stearate | 0.25 |

Extragranular

| | | |
|---|---|---|
| Glidant | Colloidal Silicon Dioxide | 0.5 |
| Lubricant | Magnesium Stearate | 0.25 |

The pharmaceutical composition preferably contains a therapeutically effective amount of tucatinib. However, in some embodiments, each individual dose contains a portion of a therapeutically effective amount of tucatinib, such that multiple doses of the composition may be required (for example, two or more tablets are required for a therapeutically effective amount). Thus, in this application when it states that the pharmaceutical composition contains a therapeutically effective amount it means that the composition may be one dose (for example, one tablet) or multiple doses (for example, two tablets). In certain embodiments, the pharmaceutical composition contains between 1 and 500 mg of tucatinib.

In certain embodiments, the pharmaceutical composition contains between about 25 and about 400 mg of tucatinib. In certain embodiments, the pharmaceutical composition contains between 25 and 400 mg of tucatinib.

In certain embodiments, the pharmaceutical composition contains between about 25 and about 100 mg (e.g., about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg) of tucatinib. In certain embodiments, the pharmaceutical composition contains between 25 and 100 mg (e.g., 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg) of tucatinib. In certain embodiments, the pharmaceutical composition contains between about 25 and about 75 mg of tucatinib. In certain embodiments, the pharmaceutical composition contains between 25 and 75 mg of tucatinib. In certain embodiments, the pharmaceutical composition contains about 50 mg of tucatinib. In certain particular embodiments, the pharmaceutical composition contains 50 mg of tucatinib. In certain of the foregoing embodiments, the pharmaceutical composition is formulated as a tablet. As a non-limiting example, the pharmaceutical composition is formulated as a tablet and contains 50 mg of tucatinib.

In certain embodiments, the pharmaceutical composition contains between about 100 and about 300 mg (e.g., about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg) of tucatinib. In certain embodiments, the pharmaceutical composition contains between 100 and 300 mg (e.g., 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg) of tucatinib. In certain embodiments, the pharmaceutical composition contains between about 100 and about 200 mg of tucatinib. In certain embodiments, the pharmaceutical composition contains between 100 and 200 mg of tucatinib. In certain embodiments, the pharmaceutical composition contains between about 125 and about 175 mg of tucatinib. In certain embodiments, the pharmaceutical composition contains between 125 and 175 mg of tucatinib. In certain embodiments, the pharmaceutical composition contains about 150 mg of tucatinib. In certain particular embodiments, the pharmaceutical composition contains 150 mg of tucatinib. In certain of the foregoing embodiments, the pharmaceutical composition is formulated as a tablet. As a non-limiting example, the pharmaceutical composition is formulated as a tablet and contains 150 mg of tucatinib.

The pharmaceutical compositions described herein may be administered by any convenient route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), ocular, vaginal, intraperitoneal, intrapulmonary and intranasal. If parenteral administration is desired, the compositions will be sterile and in a solution or suspension form suitable for injection or infusion.

The compounds may be administered in any convenient administrative form, e.g., tablets, powders, capsules, dispersions, suspensions, syrups, sprays, suppositories, gels, emulsions, patches, etc.

The pharmaceutical compositions described herein are typically administered orally. The pharmaceutical compositions described herein are typically administered as a tablet, caplet, hard or soft gelatin capsule, pill, granules or a suspension.

Additional examples of pharmaceutical compositions of tucatinib and methods of preparation thereof are described in U.S. Pat. No. 9,457,093, which is incorporated by reference herein in its entirety.

The pharmaceutical compositions described herein may comprise one or more polymorphs of tucatinib. Exemplary polymorphs of tucatinib and methods of preparation thereof are described in U.S. Pat. No. 9,168,254, which is incorporated by reference herein in its entirety.

In some embodiments, the pharmaceutical composition comprises amorphous tucatinib. In certain embodiments, tucatinib in the pharmaceutical composition is substantially amorphous (e.g., at least 80%, at least 85%, at least 90%, or at least 95% amorphous).

In some embodiments, the pharmaceutical composition comprises a crystalline polymorph of tucatinib. In certain embodiments, tucatinib in the pharmaceutical composition is substantially crystalline (e.g., at least 80%, at least 85%, at least 90%, or at least 95% crystalline).

In certain embodiments, the pharmaceutical composition comprises polymorph Form A of tucatinib as described in U.S. Pat. No. 9,168,254. In certain embodiments, tucatinib in the pharmaceutical composition is substantially in Form A (e.g., at least 80%, at least 85%, at least 90%, or at least 95% Form A).

In certain embodiments, the pharmaceutical composition comprises polymorph Form B of tucatinib as described in U.S. Pat. No. 9,168,254. In certain embodiments, tucatinib in the pharmaceutical composition is substantially in Form B (e.g., at least 80%, at least 85%, at least 90%, or at least 95% Form B).

In certain embodiments, the pharmaceutical composition comprises polymorph Form C of tucatinib as described in U.S. Pat. No. 9,168,254. In certain embodiments, tucatinib in the pharmaceutical composition is substantially in Form C (e.g., at least 80%, at least 85%, at least 90%, or at least 95% Form C).

In certain embodiments, the pharmaceutical composition comprises polymorph Form D of tucatinib as described in U.S. Pat. No. 9,168,254. In certain embodiments, tucatinib in the pharmaceutical composition is substantially in Form D (e.g., at least 80%, at least 85%, at least 90%, or at least 95% Form D).

In certain embodiments, the pharmaceutical composition comprises polymorph Form E of tucatinib as described in U.S. Pat. No. 9,168,254. In certain embodiments, tucatinib in the pharmaceutical composition is substantially in Form E (e.g., at least 80%, at least 85%, at least 90%, or at least 95% Form E).

In certain embodiments, the pharmaceutical composition comprises polymorph Form F of tucatinib as described in U.S. Pat. No. 9,168,254. In certain embodiments, tucatinib in the pharmaceutical composition is substantially in Form F (e.g., at least 80%, at least 85%, at least 90%, or at least 95% Form F).

In certain embodiments, the pharmaceutical composition comprises polymorph Form G of tucatinib as described in U.S. Pat. No. 9,168,254. In certain embodiments, tucatinib in the pharmaceutical composition is substantially in Form G (e.g., at least 80%, at least 85%, at least 90%, or at least 95% Form G).

In certain embodiments, the pharmaceutical composition comprises polymorph Form H of tucatinib as described in U.S. Pat. No. 9,168,254. In certain embodiments, tucatinib in the pharmaceutical composition is substantially in Form H (e.g., at least 80%, at least 85%, at least 90%, or at least 95% Form H).

In certain embodiments, the pharmaceutical composition comprises polymorph Form I of tucatinib as described in U.S. Pat. No. 9,168,254. In certain embodiments, tucatinib in the pharmaceutical composition is substantially in Form I (e.g., at least 80%, at least 85%, at least 90%, or at least 95% Form I).

In certain embodiments, the pharmaceutical composition comprises polymorph Form J of tucatinib as described in U.S. Pat. No. 9,168,254. In certain embodiments, tucatinib in the pharmaceutical composition is substantially in Form J (e.g., at least 80%, at least 85%, at least 90%, or at least 95% Form J).

In certain embodiments, the pharmaceutical composition comprises polymorph Form K of tucatinib as described in U.S. Pat. No. 9,168,254. In certain embodiments, tucatinib in the pharmaceutical composition is substantially in Form K (e.g., at least 80%, at least 85%, at least 90%, or at least 95% Form K).

In certain embodiments, the pharmaceutical composition comprises polymorph Form L of tucatinib as described in U.S. Pat. No. 9,168,254. In certain embodiments, tucatinib in the pharmaceutical composition is substantially in Form L (e.g., at least 80%, at least 85%, at least 90%, or at least 95% Form L).

In certain embodiments, the pharmaceutical composition comprises polymorph Form M of tucatinib as described in U.S. Pat. No. 9,168,254. In certain embodiments, tucatinib in the pharmaceutical composition is substantially in Form M (e.g., at least 80%, at least 85%, at least 90%, or at least 95% Form M).

In certain embodiments, the pharmaceutical composition comprises polymorph Form N of tucatinib as described in U.S. Pat. No. 9,168,254. In certain embodiments, tucatinib in the pharmaceutical composition is substantially in Form N (e.g., at least 80%, at least 85%, at least 90%, or at least 95% Form N).

In certain embodiments, the pharmaceutical composition comprises polymorph Form 0 of tucatinib as described in U.S. Pat. No. 9,168,254. In certain embodiments, tucatinib in the pharmaceutical composition is substantially in Form 0 (e.g., at least 80%, at least 85%, at least 90%, or at least 95% Form 0).

In certain embodiments, the pharmaceutical composition comprises polymorph Form P of tucatinib as described in U.S. Pat. No. 9,168,254. In certain embodiments, tucatinib in the pharmaceutical composition is substantially in Form P (e.g., at least 80%, at least 85%, at least 90%, or at least 95% Form P).

I. Articles of Manufacture and Kits

In another aspect, the present disclosure provides an article of manufacture or kit for treating or ameliorating the effects of breast cancer in a subject, the article of manufacture or kit comprising a pharmaceutical composition of the present disclosure (e.g., a pharmaceutical composition comprising tucatinib and an anti-HER2 antibody-drug conjugate (e.g., trastuzumab deruxtecan or ado-trastuzumab emtansine)).

The articles of manufacture or kits are suitable for treating or ameliorating the effects of breast cancers, particularly HER2 positive and/or metastatic breast cancers. In some embodiments, the cancer is an advanced cancer. In some other embodiments, the cancer is a drug-resistant cancer. In some instances, the cancer is a multidrug-resistant cancer.

Materials and reagents to carry out the various methods of the present disclosure can be provided in articles of manufacture or kits to facilitate execution of the methods. As used herein, the term “kit” includes a combination of articles that facilitates a process, assay, analysis, or manipulation. In particular, kits of the present disclosure find utility in a wide range of applications including, for example, diagnostics, prognostics, therapy, and the like.

Articles of manufacture or kits can contain chemical reagents as well as other components. In addition, the articles of manufacture or kits of the present disclosure can include, without limitation, instructions to the user, apparatus and reagents for administering combinations of tucatinib and anti-HER2 antibody-drug conjugates (e.g., trastuzumab deruxtecan or ado-trastuzumab emtansine) or pharmaceutical compositions thereof, sample tubes, holders, trays, racks, dishes, plates, solutions, buffers, or other chemical reagents. Articles of manufacture or kits of the present disclosure can also be packaged for convenient storage and safe shipping, for example, in a box having a lid.

### III. Exemplary Embodiments

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Examples

### Example 1: Tucatinib+T-DM1 Preclinical Experiments

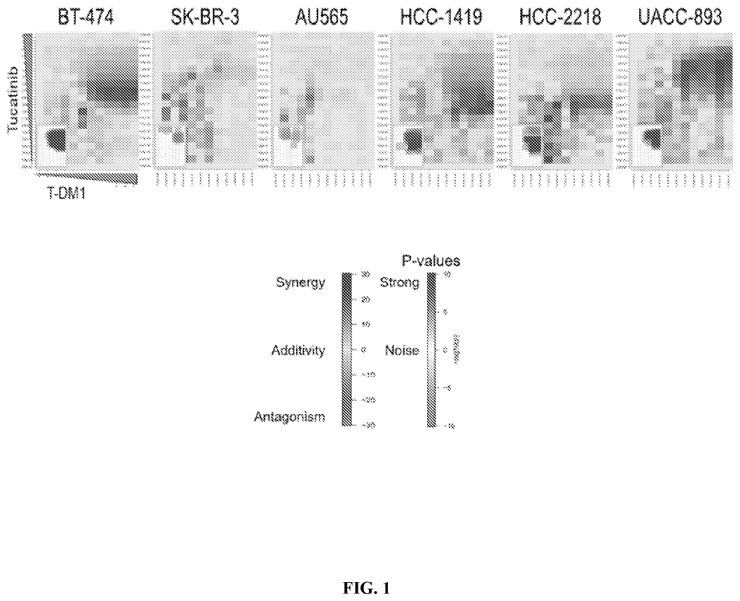

Pre-clinical data show the combination of tucatinib with T-DM1 resulted in improved antitumor activity in HER2+ breast cancer models in vitro. In HER2+ breast cancer-derived cell lines, tucatinib in combination with T-DM1 resulted in additive or synergistic activity (FIG. 1). BT-474, SK-BR-3, AU-565, HCC-1419, HCC-2218, and UACC-893 cell lines were treated with a drug concentration range spanning 0.01 nM-25 μM for tucatinib and 0.01 ng/ml-25 μg/ml for T-DM1. Cytotoxicity was evaluated at 96 hours using a CELLTITER-GLO® assay. Combinatorial activity was assessed using an HSA additivity model and the isobologram analysis was represented in heat maps. Heatmaps indicate absolute deviations from additivity for each dose combination; inset heatmaps indicate the p-values for those deviations. Isobologram analysis of the cytotoxic activity of tucatinib with T-DM1 in these HER2+ cell lines demonstrated additive activity in SK-BR-3 AU565 cell lines, while synergistic activity was observed in BT-474, HCC-1419, HCC-2218, and UACC-893 cell lines.

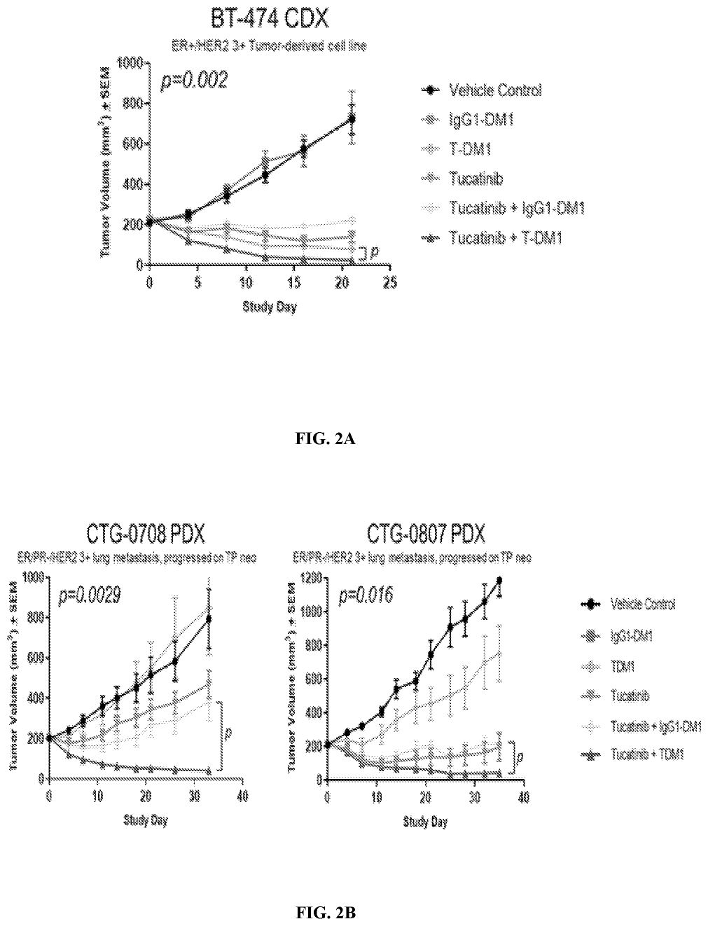

In mice bearing HER2+ subcutaneous xenografts, administration of the combination of tucatinib with T-DM1 produced increased tumor control when compared with either drug alone (FIG. 2A). Cell line-derived (CDX) BT-474 breast cancer cells were implanted subcutaneously into the flanks of female immunocompromised mice. Animals were treated with tucatinib (50 mg/kg, orally, twice daily for the duration of the study) while T-DM1 and the IgG-DM1 non-binding control ADC were dosed intravenously (10 mg/kg, single dose). Each study arm was comprised of nine animals each. Tumor volume was monitored until the designated endpoint of the study. P-values were determined by t-test analysis comparing the tucatinib/T-DM1 combination to the closest single agent arm (ref brackets). In this model, the combination of tucatinib and T-DM1 produced three complete responses and six partial responses, while tucatinib alone generated eight partial responses, and T-DM1 alone generated nine partial responses.

FIG. 2B shows the results of two HER2+ patient-derived (PDX) breast cancer models. In these T-DM1 resistant patient-derived (PDX) models, tucatinib inhibited tumor growth and the combination of tucatinib with T-DM1 produced increased tumor control when compared with either drug alone.

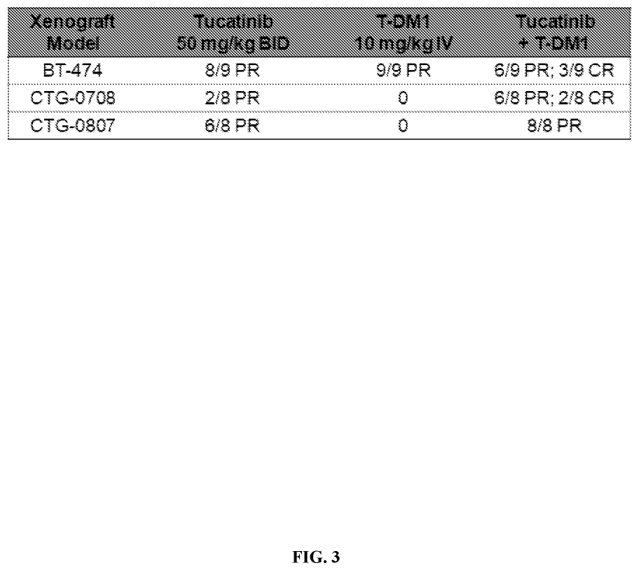

For these experiments, two T-DM1-resistant breast cancer tumor models were selected which scored 3+ by IHC and had metastasized to the lung. Tumor fragments were implanted subcutaneously into the flanks of immunocompromised mice. Animals were treated with tucatinib (50 mg/kg, orally, twice daily for the duration of the study) while T-DM1 and the IgG-DM1 non-binding control ADC were dosed intravenously (10 mg/kg, single dose). Each study arm was comprised of eight animals each. Tumor volume was monitored until the designated endpoint of the study. P-values were determined by t-test analysis comparing the tucatinib/T-DM1 combination to the closest single agent arm (ref brackets). In the CTG-0708 model, the combination of tucatinib and T-DM1 produced two complete responses and six partial responses, while tucatinib alone generated two partial responses, and T-DM1 alone generated no partial or complete responses. In the CTG-0807 model, the combination of tucatinib and T-DM1 produced 8 partial responses, while tucatinib alone generated 6 partial responses, and T-DM1 alone generated no partial or complete responses. FIG. 3 shows a summary of partial responses (PR) and complete responses (CR).

### Example 2: Phase 1b, Open-Label Study to Assess the Safety and Tolerability of Tucatinib (ONT-380) Combined with Ado-Trastuzumab Emtansine (Trastuzumab Emtansine; T-DM1)

This clinical trial evaluated the safety, tolerability, and preliminary clinical activity of tucatinib in combination with T-DM1. Study ONT-380-004 was a phase 1b, open-label, multicenter, 3+3 dose-escalation study in subjects with HER2+ mBC, designed to identify the maximum-tolerated dose (MTD) or recommended phase 2 dose (RP2D) of tucatinib in combination with T-DM1. Subjects had a history of prior therapy with trastuzumab and a taxane, separately or in combination; for subjects in the dose-escalation and MTD-expansion cohorts, prior therapy with trastuzumab and a taxane must have been for metastatic disease. For subjects in the CNS disease-expansion cohorts, trastuzumab and taxane (together or separately) might have been given at any time prior to study enrollment as part of neoadjuvant therapy, adjuvant therapy, or therapy for metastatic disease.

Fifty-seven T-DM1-naive subjects were treated (Borges 2018). The tucatinib MTD was determined to be 300 mg administered orally twice per day (PO BID) in combination with the approved dose of T-DM1 (3.6 mg/kg every 21 days). Among the 50 subjects treated at the MTD, the most common adverse events (AEs) occurring in ≥40% of subjects were nausea, diarrhea, fatigue, epistaxis, headache, vomiting, constipation, and decreased appetite; the majority of AEs were Grade 1 or 2. In these 50 subjects, the median PFS was 8.2 months (95% CI: 4.8, 10.3); the clinical benefit rate (CBR; subjects with best response of complete response (CR) or partial response [PR], or stable disease [SD] for >6 months) among 48 evaluable subjects was 58% (28 subjects). Thirty-four of 50 subjects (68%) treated with the MTD had measurable disease and were evaluable for response with an objective response rate (ORR) of 47% (1 subject with CR, 15 subjects with PR 14 subjects with SD, and 4 subjects with disease progression). Among the subjects whose disease responded to treatment, the median duration of response (DOR) was 6.9 months (95% CI: 2.8, 19.8).

Thirty of 50 subjects (60%) treated at the MTD had brain metastases at study entry. Of these, 21 of 30 subjects (70%) had either untreated or previously treated and progressive brain metastases. Median PFS among subjects with brain metastases was 6.7 months (95% CI: 4.1, 10.2). Twenty-one of these 30 subjects had measurable disease and were evaluable for response with an ORR of 48% (1 subjects with CR, 9 subjects with PR, 10 subjects with SD, and 1 subject with progressive disease). Among these subjects, the median duration of overall response was 7 months (95% CI: 1.5, NE), according to the Response Evaluation Criteria in Solid Tumors (RECIST) v1.1.

The combination of tucatinib with T-DM1 was found to have a tolerable safety profile, with evidence of clinical activity, including in subjects with brain metastases. In summary, this combination showed encouraging clinical activity with an objective response rate (ORR) of 47% (95% CI 29.8-64.9), including activity in patients with brain metastases where a brain-specific response rate was 38.5% (95% CI: 15.2-72.3), and a median PFS of 8.2 months (95% CI: 4.8-10.3). Tucatinib with T-DM1 was found to have a tolerable safety profile, and the most common adverse events were nausea (72%), diarrhea (60%), and fatigue (56%), with the majority of events being grade 1 or 2.

### Example 3: Randomized, Double-Blind, Phase 3 Study of Tucatinib or Placebo in Combination with Ado-Trastuzumab Emtansine (T-DM1) for Subjects with Unresectable Locally-Advanced or Metastatic HER2+ Breast Cancer

This example describes a double-blinded study of tucatinib or placebo in combination with ado-trastuzumab emtansine is carried out in patients with unresectable locally advanced or metastatic HER2+ breast cancer who have had had prior treatment with a taxane and trastuzumab in any setting (separately or in combination).

### Study Objectives

### Primary

Compare progression-free survival (PFS) by investigator assessment per Response Evaluation Criteria in Solid Tumors (RECIST) v1.1 between treatment arms

### Key Secondary

Compare overall survival (OS) between treatment arms

Compare the objective response rate (ORR) by investigator assessment per RECIST v1.1 between treatment arms

### Other Secondary

Evaluate PFS by blinded independent central review (BICR) per RECIST v1.1 between treatment arms

Evaluate PFS by investigator assessment per RECIST v1.1 in subjects with brain metastases at baseline between treatment arms

Evaluate PFS by BICR per RECIST v1.1 in subjects with brain metastases at baseline between treatment arms

Evaluate the ORR by BICR per RECIST v1.1 between treatment arms

Evaluate the duration of response (DOR) by investigator assessment per RECIST v1.1 between treatment arms

Evaluate the clinical benefit rate (CBR; stable disease [SD] or non-complete response [CR]/non-progressive disease [PD] for ≥6 months or best response of complete response [CR] or partial response [PR]) by investigator assessment per RECIST v1.1 between treatment arms

Evaluate the CBR by BICR per RECIST v1.1 between treatment arms

Evaluate the safety of tucatinib in combination with T-DM1

### Exploratory

Evaluate the pharmacokinetics (PK) of tucatinib and DM1 following administration of tucatinib and T-DM1 in combination

To assess exploratory biomarkers in relation to response and resistance to tucatinib

Evaluate on-trial healthcare resource utilization (HCRU) between treatment arms

Evaluate patient reported outcomes (PROs) and health-related quality of life (QoL) between treatment arms

### Study Population

Eligible subjects are at least 18 years of age and have unresectable locally-advanced or metastatic (LA/M) human epidermal growth factor receptor 2 (HER2)-positive breast cancer with a life expectancy of at least 6 months. Subjects must have histologically confirmed HER2+ carcinoma, had prior treatment with a taxane and trastuzumab in any setting (separately or in combination), and must have progressed or have been intolerant of the last systemic therapy. Hormone receptor (HR) status must also be known prior to randomization. Subjects must have an Eastern Cooperative Oncology Group (ECOG) performance status of ≤1, adequate cardiac function, and adequate renal, hepatic, and hematologic function at baseline. Prior treatment with tucatinib, T-DM1, lapatinib within 12 months of starting study treatment (except in cases where lapatinib was given for ≤21 days and was discontinued for reasons other than disease progression or severe toxicity), neratinib, afatinib, trastuzumab deruxtecan (DS8201a), or any other investigational anti-HER2 or anti-epidermal growth factor receptor (EGFR) agent or HER2 tyrosine kinase inhibitor (TKI) agent is not permitted. Prior pertuzumab therapy is allowed, but not required. Subjects must be >3 weeks post-treatment from any prior systemic anti-cancer therapy (including hormonal therapy), non-central nervous system (CNS) radiotherapy or participation in another interventional clinical trial.

Subjects with untreated brain metastases on screening brain magnetic resonance imaging (MRI) are eligible if immediate local therapy is not required. Subjects with brain metastases previously treated with local therapy are eligible if the brain metastases are stable since treatment; or, if there has been progression since the prior local CNS therapy, immediate re-treatment with local therapy is not required. If treatment for newly identified lesions is initiated, subjects may still be eligible if other sites of evaluable disease are present and treatment is completed prior to the first dose of study treatment as follows: stereotactic radiosurgery (SRS) is completed ≥7 days prior, whole brain radiation therapy is completed ≥14 days prior, or time since surgical resection is ≥28 days. Ongoing use of systemic corticosteroids at a total daily dose of >2 mg of dexamethasone (or equivalent) for symptomatic control is not permitted. Subjects with poorly controlled generalized or complex partial seizures, or manifest neurologic progression due to brain metastases notwithstanding CNS-directed therapy are not permitted.

### Number of Planned Subjects

Approximately 460 subjects (approximately 230 subjects per treatment arm) will be randomized in this study.

### Study Design

This is a randomized, double-blind, placebo-controlled, international, multicenter, phase 3 study designed to evaluate the efficacy and safety of tucatinib in combination with T-DM1 in subjects with unresectable LA/M HER2+ breast cancer who have had prior treatment with a taxane and trastuzumab in any setting. Subjects will be randomized in a 1:1 manner to receive 21-day cycles of either tucatinib or placebo in combination with T-DM1. Randomization will be stratified by line of treatment for metastatic disease, HR status, presence or history of brain metastases, and ECOG performance status.

While on study treatment, subjects will be assessed for progression every 6 weeks for the first 24 weeks, and every 9 weeks thereafter, irrespective of dose holds or interruptions. After completion of study treatment and after occurrence of disease progression, subjects in both arms of the study will continue to be followed for survival until study closure or withdrawal of consent.

An Independent Data Monitoring Committee (IDMC) will periodically review relevant aggregate safety data (blinded and unblinded) and will make recommendations to the sponsor. Safety will also be monitored in an ongoing, blinded basis by the sponsor throughout the study.

### Investigational Product, Dose, and Mode of Administration

Subjects will be randomized in a 1:1 manner to receive study treatment on a 21-day cycle, either: Control arm: Placebo given orally twice a day (PO BID); T-DM1 3.6 mg/kg given intravenously (IV) every 21 days or Experimental arm: Tucatinib 300 mg PO BID; T-DM1 3.6 mg/kg IV every 21 days.

### Duration of Treatment

Study treatment will continue until unacceptable toxicity, disease progression, withdrawal of consent, or study closure. In the absence of clear evidence of radiographic progression, development of CNS symptoms, or radiographic changes thought to pose potential immediate risk to the subject, all efforts should be made to continue treatment until unequivocal evidence of radiologic progression occurs. No crossover from placebo to tucatinib will be allowed. Subjects assessed as having isolated progression in the brain per RECIST v1.1, may be eligible to continue on study treatment for clinical benefit after undergoing local therapy to CNS disease, with approval from the medical monitor.

### Efficacy Assessments

Disease response per RECIST v1.1 (Eisenhauer 2009) will be assessed by both investigator assessment and BICR. Response assessments will include measurement of all known sites of unresectable LA/M disease (including at a minimum the chest, abdomen, and pelvis), preferably by high quality spiral contrast computed tomography (CT), at baseline, every 6 weeks for the first 24 weeks, and every 9 weeks thereafter, irrespective of dose interruptions. Positron emission tomography (PET)/CT (if high quality CT scan included), and/or Mill scan may also be done as appropriate, as well as additional imaging of any other known sites of disease (e.g., skin lesion photography for skin lesions, nuclear bone scan imaging for bone lesions).

Contrast Mill of the brain will be required on this same schedule only in those subjects with prior history of brain metastases or brain metastases found at screening. Additional contrast MRIs of the brain may also be performed in subjects without known brain metastases if there is clinical suspicion of new brain lesions.

Treatment decisions will be made based upon local assessment of radiologic scans. Response assessments for each subject will continue until a PFS event per RECIST v1.1 by investigator assessment has been documented. Follow-up for survival will continue until study closure or withdrawal of consent.

### Pharmacokinetic Assessments

PK assessments will be performed from Cycle 3 to Cycle 6 in all subjects to assess the steady state PK of tucatinib and DM1. Approximately 50 subjects (25 from each treatment arm) will participate in a PK sub-study with additional PK sampling on Days 1, 2, 3, and 5 in Cycle 2 to assess any effects of tucatinib on the PK of DM1.

### Biomarker Assessments

Blood samples will be collected predose on Cycle 1 Day 1, Cycle 3 Day 1, and at End of Treatment (EOT) to assess exploratory biomarkers in relation to response and resistance to tucatinib.

### Other Assessments—Quality of Life

Health-related QoL will be assessed at protocol-specified time points using standardized assessment tools including the European Quality of Life 5Dimension 3Level (EQ-5D-3L) instrument, the European Organization for Research and Treatment of Cancer (EORTC) quality-of-life questionnaire (QLQ-C30), National Cancer Institute's patient-reported outcomes version of the Common Terminology Criteria for Adverse Events (NCIPRO-CTCAE) questionnaire customized to focus on adverse events (AEs) or symptoms of interest, and the Functional Assessment of Cancer Therapy—Breast (FACTB).

### Safety Assessments

Safety assessments will include surveillance and recording of AEs, physical examination findings, and laboratory tests. Assessment of cardiac ejection fraction will be performed by multi-gated acquisition (MUGA) scan or echocardiogram (ECHO).

### Statistical Methods

### Stratification

Stratification factors will include line of treatment for metastatic disease (1st line vs. other), HR status (positive/negative), presence or history of treated or untreated brain metastases (yes/no), and ECOG performance status (0 vs. 1). Stratification for presence of brain metastases will be based upon medical history and investigator assessment of screening contrast brain MRI.

### Objectives and Endpoints

This study will evaluate the efficacy and safety of tucatinib versus placebo in combination with T-DM1 in subjects with unresectable locally-advanced or metastatic (LA/M) HER2+ breast cancer.

### Investigational Plan

### Summary of Study Design

This is a randomized, double-blind, placebo-controlled, international, multicenter, phase 3 study designed to evaluate the efficacy and safety of tucatinib in combination with T-DM1 in subjects with unresectable LA/M HER2+ breast cancer who have had prior treatment with a taxane and trastuzumab in any setting. Subjects will be randomized in a 1:1 manner to receive 21-day cycles of treatment in 1 of the following 2 treatment groups: Control arm: Placebo given PO BID; T-DM1 3.6 mg/kg given intravenously (IV) every 21 days and Experimental arm: Tucatinib 300 mg PO BID; T-DM1 3.6 mg/kg IV every 21 days.

Tucatinib or placebo will be dispensed to subjects in a double-blinded manner. Protocol defined visits and cycle numbering will be determined by T-DM1 dosing date, allowing for dose holds or delays with T-DM1. Study treatment will continue until unacceptable toxicity, disease progression, withdrawal of consent, or study closure. Disease response and progression will be assessed using RECIST v1.1. While on study treatment, radiographic disease evaluations will be performed every 6 weeks for the first 24 weeks, and every 9 weeks thereafter, irrespective of dose holds or interruptions. In the absence of clear evidence of radiographic progression, development of CNS symptoms, or radiographic changes thought to pose potential immediate risk to the subject, all efforts should be made to continue treatment until unequivocal evidence of radiologic progression occurs. Subjects assessed as having isolated progression in the brain per RECIST v1.1, may be eligible to continue on study treatment for clinical benefit after undergoing local therapy for CNS disease, with approval from the medical monitor.

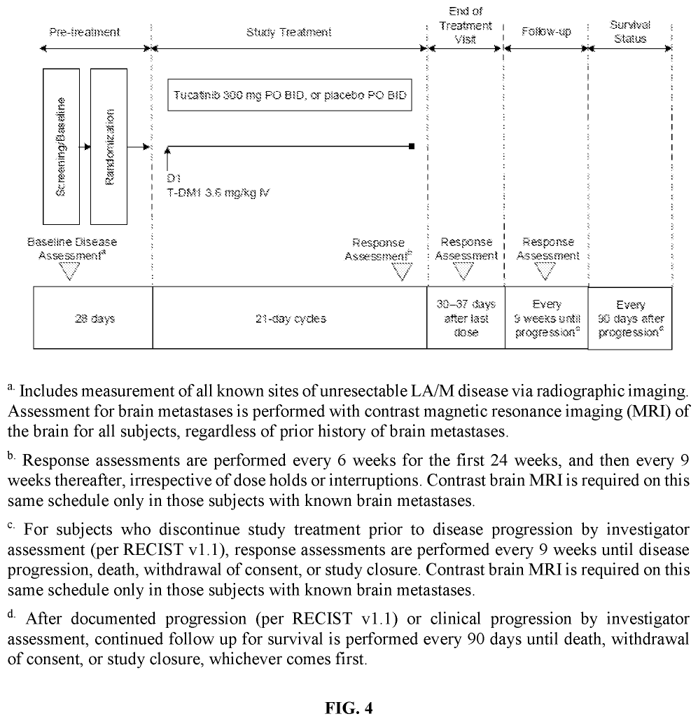

After completion of study treatment and after occurrence of disease progression, subjects in both arms of the study will continue to be followed for survival until study closure or withdrawal of consent. Safety will be monitored on a blinded basis by the sponsor throughout the study. An independent data monitoring committee (IDMC) will regularly review all relevant aggregate safety data (blinded and unblinded). Approximately 460 subjects (approximately 230 subjects per treatment arm) will be randomized in this study. A study schema is provided in FIG. 4.

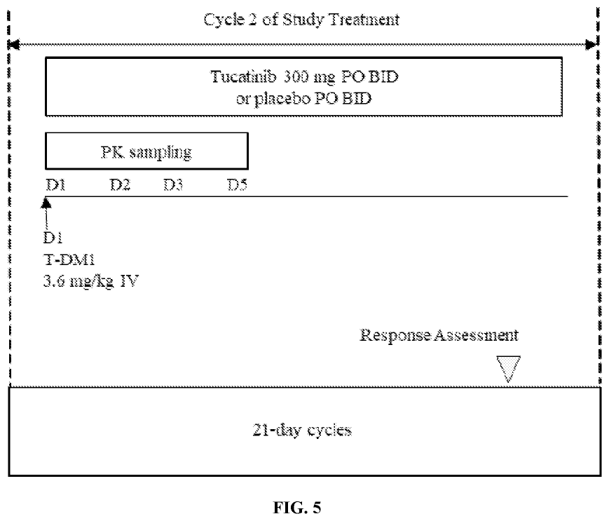

A pharmacokinetics (PK) sub-study will assess the effects of tucatinib on the PK of DM1. With additional consent, approximately 50 subjects (enrollment will continue until at least 25 subjects from each treatment arm have completed the sub-study) will participate in a PK substudy in which additional PK assessments on Days 1, 2, 3, and 5 in Cycle 2 are performed (see FIG. 5).

### Study Population

Subjects must meet all of the enrollment criteria to be eligible for this study. Eligibility criteria may not be waived by the investigator and are subject to review in the event of a good clinical practice audit and/or health regulatory authority inspection.

### Inclusion Criteria

1. Histologically confirmed HER2+ metastatic breast carcinoma, as determined by sponsordesignated central laboratory testing on tumor tissue submitted prior to randomization, from either:
   a. Archival tissue (most recent tumor tissue sample preferred)
   b. If archival tissue is not available, then a newly-obtained baseline biopsy of an accessible tumor lesion that has not been previously irradiated is required
2. History of prior treatment with a taxane and trastuzumab in any setting, separately or in combination. Prior pertuzumab therapy is allowed, but not required.
3. Have progression of unresectable LA/M breast cancer after last systemic therapy (as confirmed by investigator), or be intolerant of last systemic therapy
4. Measureable or non-measurable disease assessable by RECIST v1.1
5. HR (estrogen receptor [ER]/progesterone receptor [PR]) status must be known prior to randomization
6. Age ≥18 years at time of consent
7. ECOG performance status score of 0 or 1
8. Life expectancy ≥6 months, in the opinion of the investigator
9. Adequate hepatic function as defined by the following:
   a. Total bilirubin ≤1.5× upper limit of normal (ULN), except for subjects with known Gilbert's disease, who may enroll if the conjugated bilirubin is ≤1.5×ULN
   b. Transaminases (aspartate aminotransferase/serum glutamic oxaloacetic transaminase [AST/SGOT] and alanine aminotransferase/serum glutamic pyruvic transaminase [ALT/SGPT])≤2.5×ULN (≤5×ULN if liver metastases are present)
10. Adequate baseline hematologic parameters as defined by:
   a. Absolute neutrophil count ≥1.5×103/μL
   b. Platelet count ≥100×103/μL
   c. Hemoglobin ≥9 g/dL
   d. In subjects transfused before study entry, transfusion must be ≥14 days prior to start of therapy to establish adequate hematologic parameters independent from transfusion support
11. Estimated glomerular filtration rate (GFR) ≥50 mL/min/1.73 m2 using the Modification of Diet in Renal Disease (MDRD) study equation as applicable.
12. International normalized ratio (INR) and partial thromboplastin time (PTT)/activated partial thromboplastin time (aPTT) ≤1.5×ULN, unless on medication known to alter INR and PTT/aPTT.
13. Left ventricular ejection fraction (LVEF) ≥50% as assessed by echocardiogram (ECHO) or multi-gated acquisition scan (MUGA) documented within 4 weeks prior to first dose of study treatment
14. For subjects of childbearing potential, as defined herein, the following stipulations apply:
   a. Must have a negative serum or urine pregnancy test (minimum sensitivity of 25 mIU/mL or equivalent units of beta human chorionic gonadotropin [β-hCG]) result within 7 days prior to the first dose of study treatment. A subject with a false positive result and documented verification that the subject is not pregnant is eligible for participation.
   b. Must agree not to try to become pregnant during the study and for at least 7 months after the final dose of study drug administration
   c. Must agree not to breastfeed or donate ova, starting at time of informed consent and continuing through 7 months after the final dose of study drug administration
   d. If sexually active in a way that could lead to pregnancy, must consistently use highly effective methods of birth control (i.e., methods that achieve a failure rate of <1% per year when used consistently and correctly) starting at the time of informed consent and continuing throughout the study and for at least 7 months after the final dose of study drug administration. Highly effective methods of birth control include:
      Intrauterine device
      Bilateral tubal occlusion/ligation
      Vasectomized partner
      Sexual abstinence when it is the preferred and usual lifestyle choice of the subject
15. For subjects who can father children, the following stipulations apply:
   a. Must agree not to donate sperm starting at time of informed consent and continuing throughout the study period and for at least 7 months after the final study drug administration
   b. If sexually active with a person of childbearing potential in a way that could lead to pregnancy, must consistently use a barrier method of birth control starting at time of informed consent and continuing throughout the study and for at least 7 months after the final dose of study drug administration
   c. If sexually active with a person who is pregnant or breastfeeding, must consistently use a barrier method of birth control starting at time of informed consent and continuing throughout the study and for at least 7 months after the final dose of study drug administration
16. The subject or the subject's legally acceptable representative must provide written informed consent
17. Subject must be willing and able to comply with study procedures
18. CNS Inclusion—Based on screening contrast brain magnetic resonance imaging (MRI), subjects must have at least one of the following:
   a. No evidence of brain metastases
   b. Untreated brain metastases not needing immediate local therapy. For subjects with untreated CNS lesions >2.0 cm in diameter on screening contrast brain MRI, approval from the medical monitor is required prior to enrollment.
   c. Previously treated brain metastases
      i. Brain metastases previously treated with local therapy may either be stable since treatment or may have progressed since prior local CNS therapy, provided that there is no clinical indication for immediate re-treatment with local therapy in the opinion of the investigator
      ii. Subjects treated with CNS local therapy for newly identified lesions found on contrast brain MRI performed during screening for this study may be eligible to enroll if all of the following criteria are met:
         Time since SRS is ≥7 days prior to first dose of study treatment, time since whole-brain radiation therapy (WBRT) is ≥14 days prior to first dose of study treatment, or time since surgical resection is ≥28 days
         Other sites of evaluable disease are present
      iii. Relevant records of any CNS treatment must be available to allow for classification of target and non-target lesions

### Exclusion Criteria

1. Prior treatment with tucatinib, neratinib, afatinib, trastuzumab deruxtecan (DS-8201a), or any other investigational anti-HER2, anti-EGFR, or HER2 TKI agent. Prior treatment with lapatinib within 12 months of starting study treatment (except in cases where lapatinib was given for ≤21 days and was discontinued for reasons other than disease progression or severe toxicity)
2. Prior treatment with T-DM1
3. History of allergic reactions to trastuzumab or compounds chemically or biologically similar to tucatinib, except for Grade 1 or 2 infusion related reactions to trastuzumab that were successfully managed, or known allergy to any of the excipients in the study drugs
4. Treatment with any systemic anti-cancer therapy (including hormonal therapy), non-CNS radiation, experimental agent or participation in another interventional clinical trial ≤3 weeks prior to first dose of study treatment. An exception for the washout of hormonal therapies is gonadotropin releasing hormone agonists used for ovarian suppression in premenopausal women, which are permitted concomitant medications.
5. Any toxicity related to prior cancer therapies that has not resolved to ≤Grade 1, with the following exceptions:
   Alopecia;
   Neuropathy, which must have resolved to ≤Grade 2;
   Congestive heart failure (CHF), which must have been ≤Grade 1 in severity at the time of occurrence, and must have resolved completely
6. Clinically significant cardiopulmonary disease such as:
   Ventricular arrhythmia requiring therapy
   Symptomatic hypertension or uncontrolled asymptomatic hypertension as determined by the investigator
   Any history of symptomatic CHF, left ventricular systolic dysfunction or decrease in ejection fraction
   Severe dyspnea at rest (Common Terminology Criteria for Adverse Events [CTCAE] Grade 3 or above) due to complications of advanced malignancy or hypoxia requiring supplementary oxygen therapy
   ≥Grade 2 QTc prolongation on screening electrocardiogram (ECG)
7. Known myocardial infarction or unstable angina within 6 months prior to first dose of study treatment
8. Known carrier of Hepatitis B or Hepatitis C or has other known chronic liver disease
9. Known to be positive for human immunodeficiency virus.
10. Subjects who are pregnant, breastfeeding, or planning to become pregnant from time of informed consent until 7 months following the last dose of study drug
11. Unable to swallow pills or has significant gastrointestinal disease which would preclude the adequate oral absorption of medications
12. Use of a strong CYP3A4 or CYP2C8 inhibitor within 2 weeks, or use of a strong CYP3A4 or CYP2C8 inducer within 5 days prior to the first dose of study treatment. CYP3A4 or CYP2C8 inducers and inhibitors are also prohibited as concomitant medications within two weeks of discontinuation of tucatinib treatment. Use of sensitive CYP3A substrates should be avoided two weeks before enrollment and during study treatment.
13. Unable to undergo contrast MRI of the brain
14. Other medical, social, or psychosocial factors that, in the opinion of the investigator, could impact safety or compliance with study procedures
15. Evidence within 2 years of the start of study treatment of another malignancy that required systemic treatment
16. CNS Exclusion—Based on screening brain MRI, subjects must not have any of the following:
   a. Any untreated brain lesions >2.0 cm in size, unless approved by the medical monitor
   b. Ongoing use of systemic corticosteroids for control of symptoms of brain metastases at a total daily dose of >2 mg of dexamethasone (or equivalent). However, subjects on a chronic stable dose of ≤2 mg total daily of dexamethasone (or equivalent) may be eligible with approval of the medical monitor.
   c. Any brain lesion thought to require immediate local therapy, including (but not limited to) a lesion in an anatomic site where increase in size or possible treatmentrelated edema may pose risk to the subject (e.g., brain stem lesions). Subjects who undergo local treatment for such lesions identified by screening contrast brain MRI may still be eligible for the study based on criteria described under CNS Inclusion 17c (ii).
   d. Known or concurrent leptomeningeal disease as documented by the investigator
   e. Poorly controlled (>1/week) generalized or complex partial seizures, or manifest neurologic progression due to brain metastases notwithstanding CNS-directed therapy
      Continuation on Study Treatment after CNS-Only Progression

If a subject is found to have isolated progression in the CNS per RECIST v1.1 (including either parenchymal brain or dural metastases but not skull-based or leptomeningeal metastases) and does not have progression of disease outside the CNS, the subject may be eligible to continue on study treatment after completion of local treatment (radiotherapy or surgery) of any progressive brain/dural metastases to allow for clinical benefit. Local treatment must be completed prior to the subject's next response assessment timepoint. Subjects may continue on study treatment for clinical benefit after this PFS event in the brain, however, requires discussion with and documented approval from the study medical monitor and subjects may continue until either systemic progression or a second isolated CNS progression. The subject will remain on the same treatment arm assigned initially, and may continue on study provided the following criteria are met and the subject continues to receive clinical benefit:
The subject is not experiencing any worsening of cancer-related symptoms or signs indicating clinically significant progression of disease. Subjects who are clinically deteriorating (e.g., have a decline in ECOG or Karnofsky performance status, symptomatic rapid disease progression requiring urgent medical intervention) and unlikely to receive further benefit from continued treatment should discontinue study treatment
The subject is tolerating study drug
Review and concurrence by the medical monitor
Subject has no evidence of unequivocal systemic progression
Subject has not had a previous isolated CNS progression while on study
Subject will be re-consented prior to continuing treatment on study

Study treatment may be held up to 6 weeks to allow local CNS therapy. Longer holds must be discussed and approved by the medical monitor.

### Treatments Administered

Subjects will be randomized in a 1:1 manner to receive 1 of the following study treatments, either: Control arm: Placebo tablets PO BID, and T-DM1 3.6 mg/kg IV every 21 days, or Experimental arm: Tucatinib 300 mg PO BID, and T-DM1 3.6 mg/kg IV every 21 days

### Investigational Study Drug (Tucatinib or Placebo)

Tucatinib, the investigational agent under study in this protocol, is a kinase inhibitor that selectively inhibits HER2, and displays limited activity against the related kinase EGFR. Tucatinib and placebo are supplied as yellow oval (150 mg) or round (50 mg) capsule-shaped tablets for oral administration. Investigational study drug (tucatinib or placebo) will be supplied in a blinded manner. No treatment crossover from placebo to tucatinib will be allowed.

### Dose and Administration

The investigational study drug (tucatinib or placebo) will be administered PO BID and may be taken with or without food. Subjects will be instructed by the pharmacist or investigator as to the specific number of tablets required for each dose. At each visit during study treatment, subjects will be supplied with the appropriate number of tablets for the number of doses to be taken prior to the next scheduled visit.

Subjects will be instructed to take tucatinib/placebo tablets twice each day (once in the morning, and once in the evening) approximately 8-12 hours between doses in the same calendar day. It is recommended that if a subject misses a scheduled dose of tucatinib and less than 6 hours have passed since the scheduled dosing time, the dose should be immediately taken. It is recommended that if more than 6 hours have passed since the scheduled dosing time, the subject should not take the missed dose but should wait and take the next regularly scheduled dose. Tablets may be taken with or without food. Tablets must be swallowed whole and may not be crushed, chewed or dissolved in liquid. On the day of dosing, the individual unit dose of the tucatinib tablet may be exposed to ambient temperature for up to 6 hours prior to dose.

Complete dosing instructions will be provided to the pharmacist prior to the initiation of the study. Complete dosing instructions will also be provided to study subjects and will include the minimum times between doses, dosing in relation to meals, and instructions for missed doses. Subject compliance with investigational study drug dosing instructions will be assessed with the use of subject diaries and study drug accountability.

### T-DM1

### Description

T-DM1 (KADCYLA®) is a HER2-targeted antibody and microtubule inhibitor conjugate, which is indicated, as a single agent, for the treatment of patients with HER2+ mBC who have previously received trastuzumab and a taxane, either separately or in combination.

### Dose, Preparation, and Administration

T-DM1 3.6 mg/kg IV will be administered on Day 1 of each 21-day cycle. T-DM1 should be prepared and administered per instructions in the KADCYLA package insert. T-DM1 will be administered IV per institutional guidelines, under the direction of the investigator. Protocol-defined visits and cycle numbering will be determined by T-DM1 dosing date, allowing for dose holds or delays with T-DM1.

### Dose Modifications

Guidelines for dose modification recommendations (including dose holds, dose reduction, or discontinuation of drug) in response to potential AEs are described herein. Dose reductions or treatment interruption/discontinuation for reasons other than those expressly described herein may be made by the investigator if it is deemed in the best interest of subject safety. Whenever possible, these decisions should first be discussed with the study medical monitor.

All AEs and clinically significant laboratory abnormalities should be assessed by the investigator for relationship to tucatinib/placebo and T-DM1. An AE may be considered related to tucatinib/placebo alone, T-DM1 alone, to both drugs, or to neither. In the event that the relationship is unclear, discussion should be held with the study medical monitor, to discuss which study drug(s) should be held and/or modified.

Doses held for toxicity will not be replaced. Investigational study drug (tucatinib or placebo) or T-DM1 should be discontinued if a delay greater than 6 weeks is required due to treatment-related toxicity, unless a longer delay is approved by the study medical monitor.

In the event of isolated progression in the CNS, study treatment may be held up to 6 weeks to allow local CNS therapy. Tucatinib/placebo and T-DM1 are to be held 1 week prior to planned CNS-directed therapy. The potential for radiosensitization with tucatinib and T-DM1 is unknown. Study treatment may be reinitiated ≥7 days after completion of SRS ≥21 days after WBRT, and ≥28 days after surgical resection. Plans for holding and reinitiating study drugs before and after local therapy will require discussion with, and documented approval from, the medical monitor.

Protocol-defined visits and cycle numbering will be determined by T-DM1 dosing, allowing for dose holds or delays with T-DM1. In the event T-DM1 is discontinued but study treatment with tucatinib/placebo continues, protocol-defined visits and cycle numbering will proceed using a 21-day cycle regardless of dose holds or delays for tucatinib/placebo.

### Tucatinib or Placebo Dose Reductions

Up to 3 dose reductions of tucatinib/placebo are allowed. In the case of recurrent toxicity after 3 dose reductions, treatment with tucatinib/placebo should be discontinued.

### T-DM1 Dose Reductions

Up to 2 dose reductions of T-DM1 will be allowed.

### Concomitant Therapy

All concomitant medications, blood products, and radiotherapy administered will be recorded from Day 1 (predose) through the safety reporting period. Any concomitant medication given for a study protocol-related AE should be recorded from the time of informed consent through the safety reporting period.

### Required Concomitant Therapy

There are no required concomitant therapies. For subjects with CNS metastases, prophylactic pre-treatment systemic corticosteroids may be administered at the discretion of the investigator.

### Permitted Concomitant Therapy

Subjects may continue to use any ongoing medications not prohibited by the inclusion/exclusion criteria. However, efforts should be made to maintain stable doses of concomitant medications during the course of study treatment. Supportive treatments will be given according to label instructions as medically indicated. Concomitant medications can be administered at the investigator's discretion to conform to standard practice during the treatment period.

### Screening/Baseline Assessments

Screening/Baseline assessments will be conducted to establish study baseline status and determine study eligibility. Only subjects who meet all inclusion and exclusion criteria will be enrolled in this study.

Tumor tissue must be submitted to the sponsor-designated central laboratory for confirmatory HER2 testing to determine subject eligibility; confirmatory HER2 testing may be performed on archival tissue or a newly-obtained baseline biopsy of an accessible tumor lesion that has not been previously irradiated.

Subject medical history includes a thorough review of significant past medical history, current conditions, any treatment for prior malignancies and response to prior treatment, and any concomitant medications.

All measurable and evaluable lesions will be assessed and documented at Screening/Baseline. A contrast Mill of the brain is performed to evaluate for the presence of brain metastases. Subjects with brain metastases at study entry may be eligible for study participation if they meet the inclusion/exclusion criteria.

A physical examination including height and weight, vital signs, ECOG performance status, clinical laboratory testing and pregnancy testing will be done at Screening/Baseline.

### Confirmation of HER2 Expression for Study Eligibility

Archival or freshly-obtained tumor tissue (most recent tumor tissue sample preferred) must be submitted to the sponsor-designated central laboratory for confirmatory HER2 testing prior to randomization. The central laboratory will require sufficient tumor tissue to generate 5 unstained charged slides for HER2 expression testing. Archived tumor samples must be formalin-fixed and paraffin-embedded. If archival tissue that meets sample requirements is not available, fresh tissue from a tumor site (metastatic site preferred as applicable) suitable for biopsy must be obtained and submitted for confirmatory HER2 testing.

HER2 expression will be analyzed using FISH (DAKO pharmDx), and positivity will be assessed according to the package insert for HER2 interpretation.

A tumor suitable for biopsy should be accessible, not previously irradiated, and without contraindication to biopsy, in the opinion of the investigator. Tissue samples obtained via resection, excision, punch (skin lesions only), or core needle from a tumor site are suitable for testing. Fine needle aspiration, brushing, cell pellets from pleural effusion, forceps, and lavage samples are not acceptable. Tumor tissue should be of good quality based on total and viable tumor content; e.g., samples should contain a minimum of 100 tumor cells that preserve cellular context and tissue architecture, regardless of the needle gauge used to collect the sample or the retrieval method.

### Treatment for Brain Metastases Prior to Study Entry

Subjects with brain metastases at study entry may be eligible for study participation if they meet the eligibility criteria described in Sections 4.1 and 4.2. In order to minimize the risk of symptomatic cerebral edema in subjects with brain metastases in this study, subjects with high-risk metastases, including those requiring immediate local therapy, those with rapidly progressing lesions, those requiring corticosteroids at the start of the study (>2 mg of dexamethasone or equivalent per day) for control of CNS symptoms, and those with larger untreated lesions, are excluded from the trial. However, if these subjects are amenable to immediate CNS-directed therapy with either surgery or radiation, they may undergo local therapy and then be eligible for the trial. Under select circumstances subjects may receive corticosteroid therapy for acute management of symptomatic local edema, as long as contrast brain MM does not show clear evidence of CNS progression. All such instances require approval from the study medical monitor.

Immediate local therapy to the CNS may delay the screening process beyond the 28-day screening window, in which case the requirement for a repeat contrast MRI after completion of local therapy and prior to starting study treatment is as follows:
For subjects who receive brain radiotherapy during the screening period, the original baseline contrast brain MRI will serve as the baseline for comparison for further response assessments.
For subjects who undergo surgical resection of brain metastases during the screening period, a post-operative contrast brain MRI will be performed and will serve as the baseline for comparison for further response assessments.

For subjects with brain metastases discovered during screening or a history of brain metastases, relevant MRI brain reports and CNS treatment records should be obtained and available for CRF source verification.

### Response/Efficacy Assessments

Radiographic scans and additional imaging assessments (if applicable) will be performed at protocol-specified time points, or if disease progression is suspected. Clinical response of PD, SD, PR, or CR will be determined at each assessment according to RECIST v1.1 (Eisenhauer 2009), by the investigator and by BICR. Clinical management decisions will be based on local investigator assessment to ensure that treatment decisions are made in a timely manner; results of centralized review will not be available to investigators for clinical decision making.

All known sites of metastatic or locally advanced unresectable disease should be assessed by radiographic imaging at Screening/Baseline to document sites of extracranial disease and tumor burden. Imaging, preferably by high quality spiral contrast CT scan (with oral and/or IV contrast), should include the chest, abdomen, and pelvis, at a minimum; PET/CT (if high quality CT scan is included) and/or MRI scan may also be done as appropriate. If a CT scan with contrast is contraindicated (i.e., in subjects with contrast allergy or impaired renal clearance), a non-contrast CT scan of the chest may be performed instead, with MM scans of the abdomen and pelvis. At the investigator's discretion, other appropriate imaging (e.g., skin lesion photography for skin lesions, nuclear bone scan imaging for bone lesions) should be used to assess additional known sites of measurable disease. The same imaging modalities employed in Screening/Baseline should be used for all subsequent response assessments during study treatment and in the follow-up period, unless otherwise clinically indicated. If any other radiographic or assessment exam, including pathology from any on-study biopsies or procedures, is conducted per standard of care, the assessment information will be collected in the CRF. All imaging will be collected for retrospective BICR.

In the event of equivocal progression, for example a new lesion which is small in size (defined as an equivocal new lesion) and no imminent threat to subject safety, all efforts should be made to continue the subject until unequivocal radiologic progression or clinical progression is documented. Demonstration of an unequivocal new lesion constitutes disease progression.

Subjects' clinical data must be available for CRF source verification. Copies of tumor images must be made available for review by the sponsor (or its designee) upon request. All imaging will be submitted or uploaded for retrospective BICR as soon as reasonably possible (e.g., within approximately 2 weeks) following the date of assessment. Refer to the Study Manual for instructions on collecting and submitting tumor imaging studies to the third-party imaging core laboratory for BICR.

### Evaluation of Brain Metastases

Brain MRI imaging will be performed locally and collected prospectively for centralized independent review. However, treatment decisions will be made on the basis of local review of radiologic imaging.

Contrast MRI scan of the brain will be performed for all subjects at Screening/Baseline to assess tumor burden in the brain and/or dura and identify subjects with brain metastases at baseline. CT of the brain will not be allowed, and subjects with known contraindications to undergoing contrast MRI imaging will be excluded from the study. Subjects are considered to have brain metastases at baseline with any of the following:
Any history of brain metastases
Any brain metastases at baseline
Brain lesions of equivocal significance at baseline

Only subjects with documented brain metastases at baseline, as defined above, will continue to have follow-up contrast MRIs of the brain on the same schedule as non-CNS response assessments. Contrast MRIs of the brain may also be performed in subjects without known brain metastases if there is clinical suspicion of new brain lesions. All subjects will have an additional contrast MRI of the brain at the EOT visit, unless one has been performed within 30 days of discontinuing study treatment or the reason for going off treatment was progression in the brain.

In subjects with baseline brain lesions, at least one brain lesion should be included in the baseline RECIST lesion selection as either a target or non-target lesion. As an exception, however, when unsuspected brain metastases are discovered at screening and immediate CNS-directed therapy is administered, treated lesions should not be selected as target lesions but as non-target lesions for the purpose of disease assessment by RECIST v1.1.

Copies of brain imaging must be made available for review by the sponsor (or its designee), upon request. Copies of all brain imaging will be submitted or uploaded for retrospective BICR as soon as reasonably possible (e.g., within approximately 2 weeks) following the date of assessment. Refer to the Study Manual for instructions on collecting and submitting brain imaging studies to the third-party imaging core laboratory for BICR.

### Isolated Progression in the Brain

In subjects with isolated progression in the brain per RECIST v1.1 (including either parenchymal brain or dural metastases but not skull-based or leptomeningeal metastases) and does not have progression of disease outside the CNS, the subject may be eligible to continue on study treatment after completion of local treatment (radiotherapy or surgery) to the brain/dural metastases to allow for clinical benefit with medical monitor approval. This approach approximates standard clinical practice in this clinical scenario.

Because the primary endpoint of the study is PFS, every effort should be made to avoid radiation or surgery to target lesions in the brain in the absence of PD by RECIST v1.1 unless clinically necessary in the opinion of the investigator. Target lesions, once treated with local CNS therapy, cannot be adequately assessed for subsequent response to systemic therapy. Because of this, if a subject continues on assigned study therapy after local CNS treatment to a target lesion, special consideration must be given for evaluation of the treated target lesion and the impact on the overall RECIST v1.1 assessment.

Following CNS-directed therapy for isolated CNS disease progression, RECIST v1.1 criteria would continue to measure CNS target lesions(s) if previously identified and used in the overall estimation of the sum of diameters measuring total disease burden. However, following treatment, measurement of the treated CNS target lesion(s) would use the immediate pre-CNS treatment measurement. If a subsequent decrease in the size of a treated CNS lesion post-treatment is seen, the immediate pre-CNS treatment longest diameter would be used for RECIST measurement. Should a treated CNS lesion enlarge following CNS directed therapy that was identified as a target lesion, the new and larger longest diameter is to be used for RECIST measurement.

Additionally, treatment changes which may mimic progression will be taken into account, and subjects with possible “pseudo-progression” should continue on study until unequivocal evidence of radiographic or clinical progression is present. In the absence of clear evidence of PD (per RECIST v1.1), development of CNS symptoms or radiographic changes thought to pose potential immediate risk to subject, all efforts should be made to continue treatment until unequivocal evidence of radiologic progression occurs, as defined in RECIST v1.1.

### Pharmacokinetic Assessments

In all subjects, measurements from Cycle 3 to Cycle 6 will be obtained to assess the steadystate PK of tucatinib and DM1 (Table 8). Additional blood samples will be collected and processed for exploratory PK assessment. Additionally, approximately 50 subjects (enrollment will continue until at least 25 subjects from each treatment arm have completed the sub-study) will participate in a PK sub-study with additional PK sampling on Days 1, 2, 3, and 5 in Cycle 2 to assess tucatinib and DM1 plasma concentrations (Table 9). Additional blood samples will be collected and processed for exploratory PK assessment.

A liquid chromatography/tandem mass spectrometry assay will be used to assess plasma concentrations of tucatinib and DM1. Other assays may be performed if further characterization is required on the exploratory PK sample. Additional PK data analyses, including population PK and exploratory exposure-response analyses may be conducted; such analyses will be described in a separate analysis plan. Trough PK samples should continue to be collected on schedule regardless of dose holds or interruptions. The Cycle 3 Day 1 post-dose sample should not be collected during dose hold or interruptions.

### Biomarker Studies

To assess potential response and resistance biomarkers such as microsatellite instability (MSI), tumor mutation burden, HER2 amplification and other related mutations using liquid biopsy platforms, blood samples will be collected on Cycle 1 Day 1 (predose), Cycle 3 Day 1 (predose), and at the EOT visit from subjects. Biomarker assessments will not be used for subject selection in this study.

### Patient-Reported Outcomes

QoL questionnaires will be administered to compare improvements, deteriorations, and stabilization in health-related QoL between treatment arms. During study treatment, these questionnaires must be completed prior to evaluation by study personnel (physical examination, review of AEs) and administration of study treatment on treatment days. Questionnaires may be collected by phone once a subject experiences disease progression (per RECIST v.1.1) by investigator assessment, and is in long-term survival follow-up.

### EQ-5D-3L—Utility Measurement

The European Quality of Life (EuroQOL) 5-Dimension 3-Level (EQ-5D-3L) is a standardized instrument developed by the EuroQol Group for use as a generic, preference based measure of health-related QoL outcomes that can be used in a wide range of health conditions and treatments (van Agt 1994). The EQ-5D-3L consists of a descriptive system questionnaire and the EuroQOL (EQ) visual analog scale.

The descriptive system questionnaire assesses 5 dimensions of health, including mobility, self-care, usual activities, pain/discomfort, and anxiety/depression. Each dimension comprises 3 levels: no problems, some problems, and extreme problems. The scores on these 5 dimensions can be presented as a health profile or can be converted to a single summary index number (utility) reflecting preference compared to other health profiles. The recall time frame for the descriptive system is the day in which the questionnaire is administered. The EQ VAS records the subject's self-rated health status on a vertical VAS ranging from 0 (worst imaginable health state) to 100 (best imaginable health state) and can be used as a quantitative measure of health outcome that reflects the subject's own judgment.

### EORTC QLQ-C30

The EORTC-QLQ was developed to measure aspects of health-related QoL pertinent to subjects with a broad range of cancers who are participating in international clinical trials (Aaronson 1993; Bjordal 1994; Sneeuw 1998). The core instrument, the QLQ-C30 (Version 3.0), is a 30-item questionnaire consisting of the following:
5 functional domains (physical, role, cognitive, emotional, social)
3 symptom scales (fatigue, pain, nausea and vomiting)
Single items for symptoms (shortness of breath, loss of appetite, sleep disturbance, constipation, diarrhea) and financial impact of the disease
2 global items (health, overall QoL)

The questionnaire has been streamlined based on a patient-centric approach to minimize the number of questions being asked as part of the PRO data collection, therefore only questions 29 and 30 of questionnaire will be used for this study. The EORTC refers to the combination of questions used in this study as EORTC IL6.

### FACT-B

The Functional Assessment of Cancer Therapy—Breast (FACT-B) is a self-report instrument designed to measure multidimensional QoL in patients with breast cancer (Brady 1997). It is reliable, relates to similar measures in an expected pattern, and performs as predicted in relation to change in clinical status over time. The FACT-B was created with an emphasis on patients' values and brevity. It is written at the sixth-grade reading level, takes approximately 10 minutes to complete, and is available in nine languages. Its psychometric properties, brevity, and relevance to patients' values make its suitable for use in both research and clinical settings. The FACT-B will be collected while subjects are receiving study treatment and until the EOT visit; the instrument has been customized to minimize the number of questions be asked as part of the PRO data collection, therefore only question 5 will be used. The Functional Assessment of Chronic Illness Therapy (FACIT) organization referred to the combination of questions used in this study as the GPS.

### NCI-PRO-CTCAE

The NCI Patient Reported Outcomes-Common Terminology Criteria for Adverse Events (PRO-CTCAE) is a new patient-reported outcome (PRO) measurement system developed to characterize the frequency, severity, and interference of 78 symptomatic treatment toxicities (Smith 2016). These include symptomatic toxicities such as pain, fatigue, nausea, and cutaneous side effects such as rash and hand-foot syndrome, all toxicities that can be meaningfully reported from the patient perspective. The PRO-CTCAE measurement system consists of an item library of adverse symptoms, and a prototype electronic platform with a variety of features designed to promote integration of the PRO-CTCAE measurement system into clinical trials workflow. The system allows for data collection via the web, a hand-held computer, or an interactive voice-response system, and includes features that allow for customized PRO-CTCAE questionnaires, tailoring the schedule for data collection, as well as patient reminders and clinician alerts for severe symptoms. Development and validation of PRO-CTCAE were consistent with well-established measurement principles as well as guidelines for PROs instrument development proposed by the FDA and EMA. The development process included patients with cancer as well as professionals from the US and Europe with expertise in oncology, instrument development, clinical research and the regulatory aspects of cancer therapy development. The NCI-PRO-CTCAE will be customized to focus on symptomatic toxicities of interest in both the control and experimental arms.

### Study Endpoint Definitions

### Primary Endpoint: PFS per Investigator Assessment

PFS per investigator is defined as the time from the date of randomization to the investigator assessment of disease progression according to RECIST v1.1 or death from any cause, whichever occurs first. For subjects who continue on study treatment after isolated CNS progression per RECIST v1.1, PFS per investigator will be calculated from the date of randomization to the first (or earliest) investigator assessment of disease progression. Subjects without documentation of PD, or death at the time of analysis, will be censored at the date of the last tumor assessment with an overall response of CR, PR, SD or non-CR/non-PD. If there is no radiographic post-baseline tumor assessment, PFS will be censored at the date of randomization. Detailed methodology, including handling rules for missing assessments and censoring approaches for the analysis of PFS, is provided in the Statistical Analysis Plan (SAP).

### Key Secondary Endpoints

### Overall Survival

OS is defined as the time from randomization to death due to any cause. For a subject who is not known to have died by the end of study follow-up, observation of OS is censored on the date the subject was last known to be alive (i.e., the date of last contact). Subjects lacking data beyond the day of randomization will have their survival time censored on the date of randomization (i.e., OS duration of 1 day).

### Objective Response Rate by Investigator Assessment

ORR is defined as the proportion of subjects with confirmed CR or PR according to RECIST v1.1. Subjects whose disease response cannot be assessed will be considered as non-responders for calculating the ORR. ORR by investigator assessment is based on investigator response assessments.

### Other Secondary Endpoints

### PFS per BICR

PFS per BICR is defined as the time from the date of randomization to the centrally-reviewed documented disease progression according to RECIST v1.1 or death from any cause, whichever occurs first. Subjects without documentation of PD or death at the time of analysis, will be censored at the date of last radiographic disease assessment with an overall response of CR, PR, SD or non-CR/non-PD.

PFS per Investigator Assessment and BICR in Subjects with Brain

### Metastases at Baseline

PFS per investigator assessment and BICR in subjects with brain metastases at baseline is defined in the same manner as the primary endpoint of PFS per investigator assessment. For this endpoint, PFS per investigator assessment and BICR will be analyzed in the subset of subjects with brain metastases at baseline per the CRF.

### Objective Response Rate by BICR

ORR is defined as the proportion of subjects with CR or partial response (PR) according to RECIST v1.1. Subjects whose disease response cannot be assessed will be considered as non-responders for calculating the ORR. ORR per BICR is based on BICR response assessments.

### Duration of Response

DOR is defined as the time from first documentation of objective response (CR or PR that is subsequently confirmed) to the first documentation of disease progression per RECIST v1.1 or death from any cause, whichever occurs earlier. Only subjects with an objective response will be included in the analysis of duration of response. DOR per investigator is based on investigator response assessments and DOR per BICR is based on BICR response assessments.

### Clinical Benefit Rate

Clinical benefit rate (CBR) is defined as the proportion of subjects with stable disease (SD) or non-CR or non-PD for ≥6 months or best response of CR or PR according to RECIST v1.1. CBR per investigator is based on investigator response assessments and CBR per BICR is based on BICR response assessments.

### Exploratory Endpoints

### PK Analysis

Peripheral blood will be collected from subjects as described herein. Exploratory, predictive, and prognostic biomarkers associated with response, resistance, or safety observations will be monitored before and during treatment with tucatinib. Correlative studies will be conducted to gain a better understanding of target response relationship, predictive biomarkers, MOA, and resistance mechanisms.

### Biomarker Analysis

Biomarkers assessments in blood may include, may include measurements of tucatinib and its metabolites, baseline and drug-induced changes in microsatellite instability (MSI), HER2 amplification, genetic polymorphisms, and mutation burden in order to assess potential response and resistance biomarkers. Methods of analysis may include, but are not limited to: next generation sequencing of RNA and DNA.

### Healthcare Resource Utilization

HCRU data include healthcare encounters related to the subject's breast cancer, cancer treatment, or cancer-related assessments.

### Patient-Reported Outcomes

Changes in QoL will be measured based on PROs according to the EQ-5D-3L, EORTC, QLQC30, NCI-PRO-CTCAE, and FACTB.

### Efficacy Analyses

### Primary Efficacy Analyses

The stratified log-rank test will be used in the primary evaluation of PFS differences between the treatment arms in the ITT analysis set using a two-sided significance level of 0.05. A stratified Cox proportional-hazards (PH) model, will be used to estimate the hazard ratio and its 95% CI. Both stratified log-rank and Cox PH models will take into account the stratification factors for randomization. Please note, if the sample size of one stratum from a stratification factor is too small, statistical analysis may not include this stratification factor. The minimum sample size for a stratum to be included in the statistical model will be specified in the SAP.

All events entered in the database at the time of analysis will be included in the analysis of PFS, even if there are more than the prespecified number of events. Kaplan-Meier curves depicting PFS in the 2 treatment arms will be generated. Additionally, median PFS and the 2-sided 95% CIs for the median will be reported using the complementary log-log transformation method (Collett 1994). Detailed methodology is provided in the SAP.

### Secondary Efficacy Analyses

OS will be analyzed using similar methods used for the primary endpoint. The stratified logrank test will be used to evaluate the OS differences between the treatment arms. A stratified Cox proportional-hazards model will be used to estimate the hazard ratio and its 95% CI.

Both stratified log-rank and Cox PH models will take into account the stratification factors for randomization. Please note, if the sample size of one strata by a stratification factor is too small, statistical analysis may not include this stratification factor. The minimum sample size for a strata to be included in the statistical model will be specified in the SAP.

Kaplan-Meier methodology and Kaplan-Meier plots will be provided by treatment group using the ITT analysis set. The median OS and its two-sided 95% CI using the complementary log-log transformation method (Collett 1994) will be calculated by treatment group.

Secondary endpoints of PFS in subjects with brain metastases and PFS by BICR will be analyzed using same method used for the primary endpoint.

### Response Rates—Objective Response Rate and Clinical Benefit Rate

Data summaries for ORR will be provided for the Response Evaluable Set (subjects in ITT with measurable disease at baseline). The 95% CI of ORR will be estimated for each treatment group. Additionally, comparison of ORR between treatment groups will be conducted using two-sided Cochran-Mantel-Haenszel test controlling for the study stratification factors. A similar approach will be used for the CBR analysis, but the analysis for CBR will be applied to the ITT analysis set.

### Duration of Response

Only subjects with a confirmed response will be included in the analysis of duration of response (DOR). DOR is defined as the time from first documented objective response (CR or PR that is subsequently confirmed) to documented disease progression per RECIST v1.1 or death from any cause, whichever occurs first. DOR will be graphically described using Kaplan-Meier methodology. The median DOR and its 95% CI will be provided for the 2 treatment arms.

### Pharmacokinetic Analyses

Individual (subject) plasma tucatinib and DM1 concentrations at each sampling time will be listed; corresponding summary statistics at each sampling time will also be calculated. For subjects in the PK sub-study, summary statistics comparing the concentration time profiles of DM1 will be calculated. Additional exploratory PK analyses may be conducted. Exploratory analyses investigating the relationship between tucatinib and/or DM1 exposure and efficacy and safety endpoints may be conducted.

### Biomarker Analyses

Relationships of biomarker parameters (e.g., baseline values, absolute and relative changes from baseline) to efficacy, safety, and PK parameters will be explored. Relationships and associated data that are determined to be of interest will be summarized. Details will be described separately in the SAP or biomarker analysis plan.

### Health Outcomes Analyses

PRO assessments based on the EQ5D3L, EORTC QLQC30, NCI-PRO-CTCAE, and FACTB and HCRU data will summarized using descriptive statistics by treatment group.

PRO assessments will be analyzed to determine if treatment affects PRO scores. PRO scores will be analyzed using longitudinal models. All subscales and individual item scores will be tabulated. Descriptive summaries of observed data at each scheduled assessment timepoint may be presented. Time to deterioration will be assessed in specific pre-specified single sharitems from either the EORTC QLQ-C30 or FACT-B. Additional statistical modeling for PRO and HCRU measures may be performed separately in post hoc analyses.

### Safety Analyses

Safety is assessed through summaries of AEs, changes in laboratory test results, changes in vital signs, physical examination findings, changes in ECOG performance status, and changes in cardiac ejection fraction results. AEs will be classified by system organ class (SOC) and preferred term using the Medical Dictionary for Regulatory Activities (MedDRA); AE severities will be classified using the CTCAE criteria.

### Extent of Exposure

Duration of treatment, number of cycles, total dose and dose intensity will be summarized by treatment arm using the safety analysis set. Dose modifications will also be summarized.

### Adverse Events

An overview of AEs will provide a tabulation of the incidence of all AEs, treatment emergent AEs, treatment-related AEs, Grade 3 and higher AEs, SAEs, treatment-related SAEs, deaths, and AEs leading to study treatment discontinuation. AEs will be defined as treatment emergent if they are newly occurring or worsen following study treatment.

AEs will be listed and summarized by MedDRA preferred term, severity, and relationship to study drug. In the event of multiple occurrences of the same AE with the same preferred term in 1 subject, the AE will be counted once as the occurrence. The incidence of AEs will be tabulated by preferred term and treatment group. AEs leading to premature discontinuation of study drug will be summarized and listed in the same manner.

All collected AE data will be listed by treatment group, study site, subject number, and cycle. Separately, all serious AEs and AEs of special interest (e.g., any DILI, asymptomatic left ventricular systolic dysfunction, and/or cerebral edema) will be analogously listed.

### Deaths and Serious Adverse Events

SAEs will be listed and summarized in the same manner as all AEs. Events with a fatal outcome will be listed.

### Results

This study may meet the primary endpoint of progression-free survival (PFS), showing that tucatinib in combination with ado-trastuzumab emtasine is superior to ado-trastuzumab alone, including a reduction in the risk of disease progression or death. This study may also meet one or more key secondary endpoints, for example, an improvement in overall survival and improved PFS and reduction in disease progression in subjects with brain metastases compared to ado-trastuzumab emtasine alone. This study may also demonstrate that tucatinib in combination with ado-trastuzumab emtasine is well tolerated with a manageable safety profile.

### Example 4: Phase 2 Study of Tucatinib in Combination with Trastuzumab Deruxtecan in Subjects with Previously Treated Unresectable Locally-Advanced or Metastatic HER2+ Breast Cancer

### Study Objectives

### Primary and Secondary Objectives

TABLE 1

| Primary Objective | Corresponding Primary Endpoint |
|---|---|
| To determine the antitumor activity of tucatinib given | cORR per Response Evaluation Criteria in Solid |
| in combination with trastuzumab deruxtecan as | Tumors (RECIST) version 1.1 according to INV |
| measured by confirmed objective response rate | assessment |
| (cORR) according to investigator (INV) assessment | |

| Secondary Objectives | Corresponding Secondary Endpoints |
|---|---|
| To evaluate the antitumor activity of tucatinib given in | PFS per RECIST v1.1 according to INV assessment |
| combination with trastuzumab deruxtecan as measured | |
| by progression-free survival (PFS) according to INV | |
| assessment | |
| To evaluate the antitumor activity of tucatinib given in | DOR per RECIST v1.1 according to INV assessment |
| combination with trastuzumab deruxtecan as measured | |
| by duration of response (DOR) according to INV | |
| assessment | |
| To evaluate the antitumor activity of tucatinib given in | DCR per RECIST v1.1 according to INV assessment |
| combination with trastuzumab deruxtecan as measured | |
| by disease control rate (DCR) according to INV | |
| assessment | |
| To assess overall survival (OS) in subjects treated with | OS |
| tucatinib given in combination with trastuzumab | |
| deruxtecan | |
| To assess the safety and tolerability of tucatinib given | Type, incidence, severity, seriousness, and |
| in combination with trastuzumab deruxtecan | relatedness of adverse events (AEs) and lab |
| | abnormalities |
| | Frequency of dose modifications and treatment |
| | discontinuations |
| | Other relevant safety variables |
| Exploratory Objectives | Corresponding Exploratory Endpoints |

Efficacy

| | |
|---|---|
| To evaluate the antitumor activity of tucatinib given in | cORR per RECIST v1.1 according to ICR assessment |
| combination with trastuzumab deruxtecan according to | PFS per RECIST v1.1 according to ICR assessment |
| independent central review (ICR) assessment | DOR per RECIST v1.1 according to ICR assessment |
| | DCR per RECIST v1.1 according to ICR assessment |

Pharmacokinetic

| | |
|---|---|
| To evaluate the pharmacokinetics (PK) of tucatinib | Plasma concentrations of tucatinib |

Biomarker

| | |
|---|---|
| To explore correlations between blood-based or tissue | Potential biomarkers of response, resistance, or |
| biomarkers and clinical outcomes | toxicity from blood-based or tumor samples |

Patient Report Outcomes

| | |
|---|---|
| To assess patient-reported outcomes (PROs) | Change from baseline in PRO assessments of the |
| associated with tucatinib given in combination with | European Quality of Life 5-Dimension 5-Level (EQ- |
| trastuzumab deruxtecan | 5D-5L) |

### Number of Planned Subjects

Approximately 60-70 subjects will be enrolled in the study to ensure about 60 subjects will be treated at the safety monitoring committee (SMC) recommended dose.

### Investigational Plan

### Summary of Study Design

This is a single arm, open-label, multi-center phase 2 trial designed to assess the safety and efficacy of tucatinib in combination with trastuzumab deruxtecan for the treatment of subjects with LA/M HER2+ breast cancer with or without brain metastases, who have received 2 or more prior HER2-directed regimens in the metastatic setting.

A SMC will continuously monitor subjects for AEs, serious adverse events (SAEs), dose modifications, and laboratory abnormalities throughout the course of the study.

### Safety Lead-in

Ten subjects will be enrolled, irrespective of cohort, in the safety lead-in portion of the study and receive tucatinib 300 mg orally twice daily (PO BID) and trastuzumab deruxtecan 5.4 mg/kg via IV infusion on Day 1 of each of 21-day cycle. The subjects enrolled in the safety lead-in will undergo the same efficacy, PK, and biomarker analyses as all other subjects with the exception of an additional PK assessment performed at Cycle 1, Day 12. Once 10 subjects are enrolled in the safety lead-in, enrollment will be paused until all subjects have been followed for at least 1 cycle and a comprehensive review of the safety profile by the SMC has occurred. The SMC will make recommendations regarding continuing with enrollment if the safety and tolerability of the regimen is acceptable. If clinically significant safety events are observed at any point during the safety lead-in, enrollment will be paused until relatedness has been determined, and review by the SMC has occurred. Based on the totality of the safety data, the SMC may recommend proceeding with enrollment, evaluation of alternative dosing, or not proceeding with further enrollment. The SMC may also recommend expanding the safety lead-in to enroll up to approximately 10 additional subjects with continued monitoring for safety by the SMC.

### Post Safety Lead-in

Following the safety lead-in, enrollment will continue until approximately 60 response-evaluable subjects have been enrolled at the SMC recommended dose. There will be two cohorts in the study, one for subjects without brain metastases (Cohort A) and one for subjects with a history of brain metastases (Cohort B), with approximately 30 subjects enrolled into each cohort. All subjects, including those in the safety lead-in, treated at the SMC recommended dose, will be included in the efficacy analysis. Additional optional cohorts evaluating the combination of tucatinib and trastuzumab deruxtecan in earlier treatment lines for breast cancer, such as the first-line metastatic setting or neoadjuvant/adjuvant setting, may be added. Optional cohorts may also be opened in other malignancies, such as non-small cell lung cancer, urothelial cancer, gastric/gastroesophageal junction cancer, and colorectal cancer.

The primary endpoint of the study is cORR by investigator. Radiographic efficacy assessments will be made by the investigator, according to RECIST v1.1, with confirmation required ≥4 weeks from the first documentation of response. In addition, images will be collected by an ICR facility for possible future analysis.

Secondary efficacy endpoints include DOR, PFS, DCR and OS.

### Method of Assigning Subjects to a Treatment Group

This will be an open label, single arm study

### Study Population

This study will enroll subjects with previously treated unresectable locally advanced/metastatic HER2+ breast cancer who have received two or more prior HER2-directed regimens in the metastatic setting.

Subjects must meet all of the enrollment criteria outlined in Section 4.1 and Section 4.2 to be eligible for this study. Eligibility criteria may not be waived by the investigator and are subject to review in the event of a good clinical practice audit and/or health regulatory authority inspection.

### Investigational Products, Dose, and Mode of Administration

Subjects will receive tucatinib and trastuzumab deruxtecan in combination at the following doses (depending on the outcome of the safety lead-in, starting doses may be adjusted):

Tucatinib 300 mg PO BID on Days 1 to 21 of each 21-day cycle.

Trastuzumab deruxtecan 5.4 mg/kg IV on Day 1 of each 21-day cycle.

### Duration of Treatment

Subjects may continue on study treatment until progressive disease (PD), unacceptable toxicity, investigator or subject decision to discontinue, or study closure. All efforts should be made to continue treatment until unequivocal evidence of radiographic progression, per RECIST v1.1, occurs.

Subjects assessed as having isolated progression in the CNS per RECIST v1.1, may be eligible to continue on study treatment for clinical benefit after undergoing local therapy to CNS disease, with approval from the medical monitor.

### Efficacy Assessments

Disease response per RECIST v1.1 will be assessed by the investigator. Response assessments will include measurement of all known sites of unresectable LA/M disease (including at a minimum the chest, abdomen, and pelvis), preferably by high quality spiral contrast computed tomography (CT), at baseline, every 6 weeks for the first 24 weeks, and every 9 weeks thereafter, irrespective of dose delays. Positron emission tomography (PET)/CT (if high quality CT scan included), and/or MRI scan may also be done as appropriate, as well as additional imaging of any other known sites of disease (e.g., skin lesion photography for skin lesions, nuclear bone scan imaging for bone lesions). For each subject, the same imaging modality as used at baseline should be used throughout the study.

Contrast MRI of the brain will be required on this same schedule only in those subjects with prior history of brain metastases or brain metastases found on screening MRI. Additional contrast MRIs of the brain may also be performed in subjects without known brain metastases if there is clinical suspicion of new brain lesions.

Subjects that discontinue study treatment for reasons other than documented PD will continue to have disease assessments every 9 weeks until the occurrence of disease progression, per RECIST v1.1, death, withdrawal of consent or study closure.

Follow-up for survival and subsequent anti-cancer therapy will occur approximately every 3 months and continue until death, withdrawal of consent, lost to follow-up, or study closure.

### Biomarker Assessments

Blood samples will be collected at screening, Cycle 3 Day 1 (predose), and at End of Treatment (EOT) to assess exploratory biomarkers in relation to response, resistance, or toxicity. Biomarker assessments may include an exploratory assessment of HER2 mutations or other genetic alterations as potential biomarkers of response. Additional exploratory analyses on archival tissue including but not limited to immunohistochemistry (IHC) and next generation sequencing (NGS) analysis may be performed to interrogate biomarkers that are associated with tumor growth, survival, and resistance to targeted therapeutics. This assessment may enable the correlation of additional biomarkers with treatment outcome and may ultimately guide or refine patient selection strategies to better match tucatinib regimens with tumor phenotype/genotype in the future.

### Safety Assessments

Subjects will be assessed throughout the study for safety. Safety assessments including physical exam and collection of AEs and laboratory abnormalities will be performed at a minimum of once every 3 weeks throughout study treatment and 30 days after the last dose of study drugs. Laboratory assessments will be performed locally. During Cycle 1, an in-person safety assessment will be performed on Days 1 and 12. During Cycle 2, an in-person safety assessment will be performed on Day 1 and liver function tests will be collected on Cycle 2 Day 12. An in-person safety assessment will then be performed on Day 1 of each cycle throughout the remainder of the study or as clinically indicated. Assessment of cardiac ejection fraction will be performed by MUGA or ECHO at screening and at least once every 12 weeks thereafter until study discontinuation irrespective of dose delays or interruption, and 30 days after the last dose of study drugs (unless done within 12 weeks prior to 30-day follow-up visit).

Subjects will be monitored for signs and symptoms of ILD/pneumonitis. In cases where ILD/pneumonitis is suspected, treatment with trastuzumab deruxtecan will be interrupted, and the subject will undergo evaluation including radiographic imaging. Pulmonary consultation should also be considered. Dose modification or discontinuation of trastuzumab deruxtecan for cases of ILD/pneumonitis will be made as per package insert.

### Patient-Reported Outcomes

Patient-Reported Outcomes (PROs) will be explored with the EQ-5D-5L instrument. EQ-5D-5L will be administered at: pre-dose Cycle 1 Day 1 (C1D1), C2D1, C3D1, C4D1, every 2 cycles starting at Cycle 6 thereafter, until treatment discontinuation, PD, death, toxicity, withdrawal of consent or study closure, and at the EOT visit.

### Statistical Methods

Safety and efficacy will be assessed using descriptive statistics, including the number of observations, mean, median, standard deviation, minimum and maximum for continuous variables, and the number and percentages (of non-missing) per category for categorical variables.

The primary endpoint, cORR per investigator, is defined as the proportion of subjects with confirmed complete response (CR) or partial response (PR), per RECIST v1.1. The 2-sided 95% exact confidence interval (CI) using Clopper-Pearson method (Clopper 1934) will be calculated for the response rates.

For illustrative purposes, a summary of the expected 95% CIs for the overall study (N=60) and by cohort (N=30) is presented below, which shows reasonable precision for the estimation.

| Number of subjects | Number of Responses | ORR | 95% exact CI |
|---|---|---|---|
| 60 | 40 | 66.7% | (53.3%, 78.3%) |
| | 42 | 70.0% | (56.8%, 81.2%) |
| | 44 | 73.3% | (60.3%, 83.9%) |
| | 46 | 76.7% | (64.0%, 86.6%) |
| | 48 | 80.0% | (67.7%, 89.2%) |
| | 50 | 83.3% | (71.5%, 91.7%) |
| | 52 | 86.7% | (75.4%, 94.1%) |
| 30 | 20 | 66.7% | (47.2%, 82.7%) |
| | 22 | 73.3% | (54.1%, 87.7%) |
| | 24 | 80.0% | (61.4%, 92.3%) |
| | 26 | 86.7% | (69.3%, 96.2%) |

### Inclusion Criteria

Subjects must meet the following criteria to be eligible for the study:
1. Have confirmed HER2-positive breast cancer, as defined by the current American Society of Clinical Oncology—College of American Pathologists (ASCO/CAP) guidelines, previously determined at a Clinical Laboratory Improvements Amendments (CLIA)-certified or International Organization for Standardization (ISO)-accredited laboratory.
2. Have received 2 or more prior anti-HER2-based regimens in the metastatic setting
3. Have progression of unresectable LA/M breast cancer after last systemic therapy (as confirmed by investigator), or be intolerant of last systemic therapy
4. Have measurable disease assessable by RECIST v1.1
5. Be at least 18 years of age at time of consent
6. Have Eastern Cooperative Oncology Group Performance Status (ECOG PS) 0 or 1
7. Have a life expectancy of at least 6 months, in the opinion of the investigator
8. Have adequate hepatic function as defined by the following:
   a. Total bilirubin ≤1.5×upper limit of normal (ULN). Exception: Subjects with known history of Gilbert's Syndrome who have a direct bilirubin ≤1.5×ULN in addition to a normal AST and ALT are eligible.
   b. Transaminases (AST and ALT)≤2.5×ULN (≤5×ULN if liver metastases are present)
9. Have adequate baseline hematologic parameters as defined by:
   a. Absolute neutrophil count (ANC)≥1.5×10³/μL
   b. Platelet count ≥100×10³/μL
   c. Hemoglobin ≥9 g/dL
   d. In subjects transfused before study entry, transfusion must be ≥14 days prior to start of therapy to establish adequate hematologic parameters independent from transfusion support
10. Estimated glomerular filtration rate (eGFR) ≥50 mL/min/1.73 m² using the Modification of Diet in Renal Disease (MDRD) study equation
11. International normalized ratio (INR) and partial thromboplastin time (PTT)/activated partial thromboplastin time (aPTT) ≤1.5×ULN, unless on medication known to alter INR and PTT/aPTT.
12. Have left ventricular ejection fraction (LVEF) ≥50% as assessed by echocardiogram (ECHO) or multiple-gated acquisition scan (MUGA) documented within 4 weeks prior to first dose of study treatment
13. For subjects of childbearing potential, the below stipulations apply.
   a. Must have a negative serum or urine pregnancy test (minimum sensitivity of 25 mIU/mL or equivalent units of beta human chorionic gonadotropin [β-hCG]) result within 7 days prior to starting study treatment. A subject with a false positive result and documented verification that the subject is not pregnant is eligible for participation.
   b. Must agree not to try to become pregnant during the study and for at least 7 months after the final dose of study drug
   c. Must agree not to breastfeed or donate ova, starting at time of informed consent and continuing through 7 months after the final dose of study drug
   d. If sexually active in a way that could lead to pregnancy, must consistently use 2 highly effective methods of birth control starting at the time of informed consent and continuing throughout the study and for at least 7 months after the final dose of study drug administration.
14. For subjects who can father children, the following stipulations apply:
   a. Must agree not to donate sperm starting at time of informed consent and continuing throughout the study period and for at least 4 months after the final study drug
   b. If sexually active with a person of childbearing potential in a way that could lead to pregnancy, must consistently use 2 highly effective methods of birth control starting at time of informed consent and continuing throughout the study and for at least 4 months after the final dose of study drug
   c. If sexually active with a person who is pregnant or breastfeeding, must consistently use one of 2 contraception options starting at time of informed consent and continuing throughout the study and for at least 4 months after the final dose of study drug administration
15. Subject must provide signed informed consent per a consent document that has been approved by an institutional review board or independent ethics committee (IRB/IEC) prior to initiation of any study-related tests or procedures that are not part of standard-of-care for the patient's disease
16. Subjects must be willing and able to comply with study procedures
   CNS Inclusion—Based on medical history and screening contrast brain magnetic resonance imaging (MRI), subjects with a history of brain metastases must have one of the following
1. Untreated brain metastases not needing immediate local therapy. For subjects with untreated CNS lesions >2.0 cm on screening contrast brain MRI, discussion with and approval from the medical monitor is required prior to enrollment
2. Previously treated brain metastases
   a. Brain metastases previously treated with local therapy may either be stable since treatment or may have progressed since prior local CNS therapy, provided that there is no clinical indication for immediate re-treatment with local therapy in the opinion of the investigator
   b. Subjects treated with CNS local therapy for newly identified lesions or previously treated progressing lesions found on contrast brain MRI performed during screening for this study may be eligible to enroll if all of the following criteria are met:
      i. Time since whole brain radiation therapy (WBRT) is ≥14 days prior to first dose of study treatment, time since SRS is ≥7 days prior to first dose of study treatment, or time since surgical resection is ≥28 days
      ii. Other sites of measurable disease by RECIST v1.1 are present
   c. Relevant records of any CNS treatment must be available to allow for classification of target and non-target lesions

### Exclusion Criteria

Subjects will be excluded from the study for any of the following reasons:
1. Have previously been treated with:
   a. Lapatinib or neratinib within 12 months of starting study treatment (except in cases where lapatinib or neratinib was given for ≤21 days and was discontinued for reasons other than disease progression or severe toxicity)
   b. Tucatinib or any investigational HER2/EGFR or HER2 TKI (e.g. afatinib) at any time previously
   c. Trastuzumab deruxtecan or another ADC consisting of an exatecan derivative
2. History of exposure to the following cumulative doses of anthracyclines:
   a. Doxorubicin >360 mg/m²
   b. Epirubicin >720 mg/m²
   c. Mitoxantrone >120 mg/m²
   d. Idarubicin >90 mg/m²
   e. Liposomal doxorubicin (e.g. Doxil, Caelyx, Myocet)>550 mg/m²
3. History of allergic reactions to trastuzumab or compounds chemically or biologically similar to tucatinib or trastuzumab deruxtecan, except for Grade 1 or 2 infusion-related reactions (IRRs) to trastuzumab that were successfully managed, or known allergy to one of the excipients in the study drugs
4. Have received treatment with:
   a. Any systemic anti-cancer therapy (including hormonal therapy) or experimental agent ≤21 days of first dose of study treatment or are currently participating in another interventional clinical trial. An exception for the washout of hormonal therapies is gonadotropin releasing hormone (GnRH) agonists used for ovarian suppression in premenopausal women, which are permitted concomitant medications
   b. Treatment with non-CNS radiation ≤7 days prior to first dose of study treatment
   c. Major surgery within ≤28 days of first dose of study treatment
5. Have any toxicity related to prior cancer therapies that has not resolved to ≤Grade 1, with the following exceptions:
   Alopecia
   Neuropathy, which must have resolved to ≤Grade 2
   Congestive heart failure (CHF), which must have been ≤Grade 1 in severity at the time of occurrence, and must have resolved completely
   Anemia, which must have resolved to ≤Grade 2
6. Have clinically significant cardiopulmonary disease such as:
   Ventricular arrhythmia requiring therapy
   Symptomatic hypertension or uncontrolled hypertension as determined by investigator
   Any history of symptomatic CHF
   Severe dyspnea at rest (CTCAE Grade 3 or above) due to complications of advanced malignancy
   Hypoxia requiring supplementary oxygen therapy
   Have had a history of ILD/pneumonitis (e.g. interstitial pneumonia, pneumonitis, pulmonary fibrosis, or radiation pneumonitis) that required systemic corticosteroids, or has current ILD/pneumonitis, or where suspected ILD/pneumonitis cannot be ruled out by imaging at screening
7. Have known myocardial infarction or unstable angina within 6 months prior to first dose of study treatment
8. Known to be positive for hepatitis B by surface antigen expression. Known to be positive for hepatitis C infection. Subjects who have been treated for hepatitis C infection are permitted if they have documented sustained virologic response of 12 weeks
9. Presence of known chronic liver disease
10. Known to be positive for HIV
11. Active or uncontrolled clinically serious infection
12. Are pregnant, breastfeeding, or planning a pregnancy
13. Have inability to swallow pills or significant gastrointestinal disease which would preclude the adequate oral absorption of medications
14. Have used a strong cytochrome P450 (CYP) 2C8 inhibitor within 3 elimination half-lives of the inhibitor, or have used a strong CYP3A4 or moderate/strong CYP2C8 inducer within 5 days prior to first dose of study treatment.
15. Unable for any reason to undergo contrast MRI of the brain
16. Have any other medical, social, or psychosocial factors that, in the opinion of the investigator, could impact safety or compliance with study procedures
17. History of malignancy other than breast cancer within 2 years prior to screening, with the exception of those with a negligible risk of metastasis or death (e.g., 5-year OS of ≥90%), such as adequately treated carcinoma in situ of the cervix, non-melanoma skin carcinoma, localized prostate cancer, ductal carcinoma in situ, or Stage I uterine cancer.
   CNS Exclusion—Based on medical history and screening contrast brain MRI, subjects must not have any of the following:
1. Any untreated brain lesions >2.0 cm in size, unless discussed with medical monitor and approval for enrollment is given
2. Ongoing use of systemic corticosteroids for control of symptoms of brain metastases at a total daily dose of >2 mg of dexamethasone (or equivalent). However, subjects on a chronic stable dose of ≤2 mg total daily dose of dexamethasone (or equivalent) may be eligible with discussion and approval by the medical monitor
3. Any brain lesion thought to require immediate local therapy, including (but not limited to) a lesion in an anatomic site where increase in size or possible treatment-related edema may pose risk to subject (e.g. brain stem lesions). Subjects who undergo local treatment for such lesions identified by screening contrast brain MRI may still be eligible for the study based on criteria described under CNS inclusion criteria 18b
4. Known or suspected leptomeningeal disease (LMD) as documented by the investigator
5. Have poorly controlled (>1/week) generalized or complex partial seizures, or manifest neurologic progression due to brain metastases

### Treatments Administered

Subjects will receive tucatinib and trastuzumab deruxtecan in combination at the following doses (depending on the outcome of the safety lead-in, starting doses may be adjusted):
Tucatinib 300 mg PO BID on Days 1 to 21 of each 21-day cycle.
Trastuzumab deruxtecan 5.4 mg/kg IV on Day 1 of each 21-day cycle.

### Tucatinib

Tucatinib is a kinase inhibitor that selectively inhibits HER2 and displays limited activity against the related kinase EGFR. Tucatinib is supplied as coated yellow oval-shaped tablets (150 mg) or round tablets (50 mg) for oral administration. Detailed information describing the preparation, administration, and storage of tucatinib is located in the Pharmacy Instructions.

Tucatinib drug product is supplied as both a coated yellow oval-shaped tablet in a 150 mg dosage strength and a coated yellow round tablet in a 50 mg dosage strength. The tablets are manufactured from a drug product intermediate amorphous dispersion of tucatinib in polyvinylpyrrolidone-vinyl acetate copolymer, which is then combined with the pharmaceutical excipients (copovidone, crospovidone, sodium chloride, potassium chloride, sodium bicarbonate, colloidal silicon dioxide, magnesium stearate, and microcrystalline cellulose) and compressed into tablets.

### Dose and Administration

Tucatinib will be administered PO BID and may be taken with or without food. Dose modifications of tucatinib are described in Section [615]. Subjects will be instructed by the pharmacist or investigator as to the specific number of tablets required for each dose. At each visit during study treatment, subjects will be supplied with the appropriate number of tablets for the number of doses to be taken prior to the next scheduled visit.

Subjects will be instructed to take tucatinib twice each day (once in the morning, and once in the evening) approximately 8 to 12 hours between doses in the same calendar day. It is recommended that if a subject misses a scheduled dose of tucatinib and less than 6 hours have passed since the scheduled dosing time, the dose should be immediately taken. It is recommended that if more than 6 hours have passed since the scheduled dosing time, the subject should not take the missed dose but should wait and take the next regularly scheduled dose. Tucatinib may be taken with or without food. Tablets must be swallowed whole and may not be crushed, chewed or dissolved in liquid. On the day of subjects and will include the minimum times between doses, dosing in relation to meals, and instructions for missed doses.dosing, the individual unit dose of the tucatinib tablet may be exposed to ambient temperature for up to 6 hours prior to dose.Complete dosing instructions will be provided to the pharmacist prior to the initiation of the study. Complete dosing instructions will also be provided to study.

Subject compliance with study drug dosing instructions will be assessed with the use of study drug accountability. Subject diaries may also be used to assess compliance.

### Overdose

In the event of an overdose of tucatinib, defined as any dose greater than the prescribed dose, study personnel should:

Care for and medically stabilize the subject until there is no immediate risk of complications or death, if applicable. There is currently no known antidote for an overdose of tucatinib.

Notify the medical monitor as soon as they become aware of the overdose, to discuss details of the overdose (e.g., exact amount of tucatinib administered, subject weight) and AEs, if any.

### Combination Study Drug (Trastuzumab Deruxtecan)

### Description

Trastuzumab deruxtecan is an ADC consisting of a HER2-directed antibody, a topoisomerase inhibitor, and a tetrapeptide linker which is indicated, as a single agent, for treatment of patients with unresectable or metastatic HER2+ breast cancer who have received 2 or more anti-HER2-based regimens in the metastatic setting.

### Method of Procurement

Trastuzumab deruxtecan is commercially available and details regarding sourcing of trastuzumab deruxtecan may vary by site and/or region as outlined in other documents such as Clinical Trial Agreements.

### Dose, Preparation, and Administration

Trastuzumab deruxtecan (5.4 mg/kg) will be given as an IV infusion once every 21 days (Day 1 of each 21-day cycle). Trastuzumab deruxtecan should be prepared and administered per instructions in the ENHERTU package insert. Trastuzumab deruxtecan will be administered IV per institutional guidelines, under the direction of the investigator.

Protocol-defined visits and cycle numbering will be determined by trastuzumab deruxtecan dosing date, allowing for dose holds or delays with trastuzumab deruxtecan. Dose modifications of trastuzumab deruxtecan are described in Section 0.

### Overdose

For this trial, an overdose will be defined as any dose at least 10% greater than the prescribed dose of trastuzumab deruxtecan. In the event of an overdose, study personnel should:

Care for and medically stabilize the subject until there is no immediate risk of complications or death, if applicable. There is currently no known antidote for an overdose of trastuzumab deruxtecan. In the event of overdose, subjects should be observed, and appropriate supportive care should be given, if required. Notify the medical monitor as soon as they become aware of the overdose, to discuss details of the overdose (e.g., exact amount of trastuzumab deruxtecan administered, subject weight) and AEs, if any.

### Dose Modifications

Tucatinib and trastuzumab deruxtecan dose-reduction recommendations are described in Table 1 and Table 2, respectively.

Guidelines for dose modification recommendations (including dose holds, dose reduction, or discontinuation of drug) in response to potential AEs are described in the tables in Section 0a. Dose reductions or treatment interruption/discontinuation for reasons other than those described in Section 0 may be made by the investigator if it is deemed in the best interest of subject safety. Whenever possible, these decisions should first be discussed with the study medical monitor.

All AEs and clinically significant laboratory abnormalities should be assessed by the investigator for relationship to tucatinib and trastuzumab deruxtecan. An AE may be considered related to tucatinib alone, trastuzumab deruxtecan alone, to both drugs, or to neither. In the event that the relationship is unclear, discussion should be held with the study medical monitor, to discuss which study drug(s) should be held and/or modified.

Doses held for toxicity will not be replaced. Tucatinib or trastuzumab deruxtecan should be discontinued if a delay greater than 6 weeks is required due to treatment-related toxicity, unless a longer delay is approved by the study medical monitor.

In the event of isolated progression in the CNS, study treatment may be held up to 6 weeks to allow local CNS therapy. Tucatinib and trastuzumab deruxtecan are to be held 1 week prior to planned CNS-directed therapy. The potential for radiosensitization with tucatinib and trastuzumab deruxtecan is unknown. Study treatment may be reinitiated ≥7 days after completion of SRS, ≥14 days after WBRT, and ≥28 days after surgical resection. Plans for holding and reinitiating study drugs before and after local therapy will require discussion with, and documented approval from, the medical monitor.

Protocol-defined visits and cycle numbering will be determined by trastuzumab deruxtecan dosing, allowing for dose holds or delays with trastuzumab deruxtecan. In the event trastuzumab deruxtecan is discontinued but study treatment with tucatinib continues, protocol-defined visits and cycle numbering will proceed using a 21-day cycle regardless of dose holds or delays for tucatinib.

### Tucatinib Dose Reductions

Up to 3 dose reductions of tucatinib are allowed (Table 1); fewer dose reduction levels may be available if alternative tucatinib doses or schedules are adopted, following SMC recommendation. Subjects who would require a dose reduction to below 150 mg BID should discontinue treatment with tucatinib. Dose reductions of larger intervals than those described in Table 1 may be made at the discretion of the investigator, but dose reductions to below 150 mg BID are not allowed.

Tucatinib dose should not be re-escalated after a dose reduction is made.

TABLE 1
Recommended tucatinib dose reduction

| Dose Reduction Schedule | Tucatinib Dose Level^{a} |
|---|---|
| Starting dose | 300 mg PO BID^{b} |
| 1st dose reduction | 250 mg PO BID |
| 2nd dose reduction | 200 mg PO BID |
| 3rd dose reduction | 150 mg PO BID |
| Requirement for further dose reduction | Discontinue treatment |

^{a}Dose reductions of greater intervals than those recommended in this table (i.e., more than 50 mg per dose reduction) may be made if considered clinically appropriate by the investigator. However, tucatinib may not be dose reduced below 150 mg BID.
^{b}Depending on the outcome of safety lead-in, 300 mg PO BID may not be the starting dose

### Trastuzumab Deruxtecan Dose Reductions

Up to 2 dose reductions of trastuzumab deruxtecan will be allowed.

Trastuzumab deruxtecan dose should not be re-escalated after a dose reduction is made as shown in Table 2.

TABLE 2
Recommended trastuzumab deruxtecan dose reduction

| Dose Reduction Schedule | Trastuzumab Deruxtecan Dose Level |
|---|---|
| Starting dose | 5.4 mg/kg IV^{a} |
| 1st dose reduction | 4.4 mg/kg IV |
| 2nd dose reduction | 3.2 mg/kg IV |
| 3rd dose reduction | Discontinue treatment |

^{a}Depending on the outcome of safety lead-in, 5.4 mg/kg IV may not be the starting dose

### Dose Modifications for Adverse Events

### General Guidelines

General dose modification guidelines for tucatinib and trastuzumab deruxtecan are provided in Table 3 for clinical AEs.

Separate dose modification guidelines are provided for AEs of hepatotoxicity (Table 4), ILD/pneumonitis (Table 5), neutropenia and febrile neutropenia (Table 6), and LVEF decrease (Table 7).

TABLE 3
Dose modifications for clinical adverse events related to either tucatinib or trastuzumab deruxtecan

| | Tucatinib | Trastuzumab Deruxtecan |
|---|---|---|
| Clinical adverse event | Related to tucatinib | Related to trastuzumab deruxtecan |
| ≥Grade 3 AEs other than Grade 3 | Hold until severity ≤ Grade 1 or | Do not administer until severity ≤ |
| fatigue lasting ≤ 3 days; alopecia^{a}; | pretreatment level. | Grade 1 or pretreatment level. |
| nausea; vomiting; diarrhea; rash; | Restart at next lowest dose level. | Reduce to next lowest dose level. |
| correctable electrolyte abnormalities | | |
| Grade 3 nausea, vomiting or | Hold until severity ≤ Grade 1 or | Do not administer until severity ≤ |
| diarrhea WITHOUT maximal use of | pretreatment level. | Grade 1 or pretreatment level. |
| antiemetics or antidiarrheals | Initiate appropriate therapy. | Initiate appropriate therapy. |
| | Restart without dose reduction. | Optional dose reduction to next |
| | | lowest dose level. |
| Grade 3 nausea, vomiting or | Hold until severity ≤ Grade 1 or | Do not administer until severity ≤ |
| diarrhea WITH maximal use of | pretreatment level. | Grade 1 or pretreatment level. |
| antiemetics or antidiarrheals | Restart at next lowest dose level. | Optional dose reduction to next |
| | | lowest dose level. |
| Grade 4 vomiting or diarrhea | Permanently discontinue. | Permanently discontinue. |
| regardless of use of anti-emetics or | | |
| anti-diarrheals | | |
| Grade 3 rash WITHOUT maximal | Hold until severity ≤ Grade 1 or | Do not administer until severity ≤ |
| use of topical corticosteroids or anti- | pretreatment level. | Grade 1 or pretreatment level. |
| infectives | Initiate appropriate therapy. | Initiate appropriate therapy. |
| | Restart without dose reduction. | Optional dose reduction to next |
| | | lowest dose level. |
| Grade 3 rash WITH maximal use of | Hold until severity ≤ Grade 1 or | Do not administer until severity ≤ |
| topical corticosteroids or anti- | pretreatment level. | Grade 1 or pretreatment level. |
| infectives | Restart at next lowest dose level. | Optional dose reduction to next |
| | | lowest dose level. |
| Grade 4 rash regardless of use of | Permanently discontinue. | Permanently discontinue. |
| topical corticosteroids or anti- | | |
| infectives | | |

^{a}No dose modifications are required for alopecia

### Hepatotoxicity

Dose modification is required in the case of liver function abnormalities, regardless of relationship to tucatinib as outlined in Table 4.

For subjects with documented Gilbert's disease, contact the medical monitor for guidance regarding dose modifications.

TABLE 4
Dose modification guidelines for liver function abnormalities

| | Tucatinib | Trastuzumab deruxtecan |
|---|---|---|
| ALT or AST (>3-≤5 x ULN) | Dose modification not required. | Dose modification not required. |
| Bilirubin (>1.5-≤3 x ULN) | Hold until recovery to (≤1.5 x | Dose modification not required. |
| | ULN). Then resume tucatinib at the | |
| | same dose level, | |
| ALT or AST (>5-≤20 x ULN) | Hold until recovery to (≤3 x ULN) | Dose modification not required. |
| | or until return to pre-treatment level | |
| | in subjects with known liver | |
| | metastasis. Then resume tucatinib at | |
| | the next lower dose level. | |
| Bilirubin (>3-≤10 x ULN) | Hold until recovery to (<≤1.5 x | Dose modification not required. |
| | ULN). Then resume tucatinib at the | |
| | next lower dose level. | |
| ALT or AST (>20 x ULN) | Permanently discontinue | Dose modification not required. |
| OR | | |
| Bilirubin (>10 x ULN) | | |
| ALT or AST > 3 x ULN | Permanently discontinue | Dose modification not required. |
| AND | | |
| Bilirubin > 2 x ULN | | |

Source: TUKYSA Prescribing Information, Seattle Genetics, Inc., April, 2020; ENHERTU Prescribing Information Daiichi Sankyo, Inc., December 2019.

### Interstitial Lung Disease/Pneumonitis

Dose modifications are required for ILD/pneumonitis, regardless of relationship to trastuzumab deruxtecan (Table 5).

Dose modification of tucatinib is not required for ILD/pneumonitis.

TABLE 5
Dose modification guidelines for interstitial lung disease/pneumonitis

| | Tucatinib | Trastuzumab Deruxtecan |
|---|---|---|
| Asymptomatic | ILD/pneumonitis Dose modification not required | Interrupt until resolved to Grade 0, |
| (Grade 1) | | then: |
| | | if resolved in 28 days or less from |
| | | date of onset, maintain dose, |
| | | if resolved in greater than 28 days |
| | | from date of onset, reduce dose one |
| | | level (see Table 2). |
| | | consider corticosteroid treatment as |
| | | soon as ILD/pneumonitis is |
| | | suspected. |
| Symptomatic | ILD/pneumonitis Dose modification not required | Permanently discontinue |
| (Grade 2 | or greater) | Promptly initiate corticosteroid |
| | | treatment as soon as |
| | | ILD/pneumonitis is suspected. |

Source: IUKYSA Prescribing Information, Seattle Genetics, Inc., April, 2020; ENHERTU Prescribing Information Daiichi Sankyo, Inc., December 2019.

### Neutropenia and Febrile Neutropenia

Dose modifications are required for neutropenia or febrile neutropenia, regardless of relationship to trastuzumab deruxtecan (Table 6).

Dose modification of tucatinib is not required for neutropenia or febrile neutropenia.

TABLE 6
Dose modification guidelines for neutropenia and febrile neutropenia

| | Tucatinib | Trastuzumab deruxtecan |
|---|---|---|
| Grade 3 neutropenia (less than 1.0 to | Dose modification not required | Interrupt until resolved to Grade 2 or |
| 0.5 × 10⁹/L) | | less, then maintain dose |
| Grade 4 neutropenia (less than 0.5 × | Dose modification not required | Interrupt until resolved to Grade 2 or |
| 10⁹/L) | | less. Reduce dose by 1 level (see |
| | | Table 2) |
| Febrile Neutropenia (Absolute | Dose modification not required | Interrupt until resolved. Reduced |
| neutrophil count of less than 1.0 × | | dose by 1 level (see Table 2) |
| 10⁹/L and temperature greater than | | |
| 38.3° C. or a sustained temperature of | | |
| 38° C. or greater for more than 1 | | |
| hour) | | |

Source: TUKYSA Prescribing Information, Seattle Genetics, Inc., April, 2020; ENHERTU Prescribing Information Daiichi Sankyo, inc., December 2019.

### Left Ventricular Ejection Fraction Decrease

Dose modification guidelines for LVEF decrease, regardless of relationship to trastuzumab deruxtecan, are provided in Table 7.

Dose modification of tucatinib is not required for LVEF decrease.

TABLE 7
Dose modification guidelines for left ventricular ejection fraction decrease

| | Tucatinib | Trastuzumab Deruxtecan |
|---|---|---|
| LVEF greater than 45% and absolute Dose modification not required | | Dose modification not required |
| decrease from baseline is 10% to | | |
| 20% | | |
| LVEF 40% to | And absolute Dose modification not required | Dose modification not required |
| 45% | decrease from | Repeat LVEF assessment within 3 weeks |
| | baseline is less | |
| | than 10% | |
| | And absolute Dose modification not required | Interrupt treatment |
| | decrease from | Repeat LVEF assessment within 3 weeks. |
| | baseline is 10% | If LVEF has not recovered to within 10% |
| | to 20% | of baseline, permanently discontinue |
| | | treatment |
| | | If LVEF recovers to within 10% from |
| | | baseline, resume treatment at the same |
| | | dose. |

| | |
|---|---|
| LVEF less than 40% or absolute Dose modification not required | Interrupt treatment |
| decrease from baseline is greater | Repeat LVEF assessment within 3 weeks. |
| than 20% | If LVEF of less than 40% or absolute |
| | decrease from baseline of greater than 20% |
| | is confirmed, permanently discontinue |
| | treatment |
| Symptomatic congestive heart Dose modification not required | Permanently discontinue treatment |
| failure (CHF) | |

Source: TUKYSA Prescribing Information, Seattle Genetics, Inc., April, 2020; ENHERTU Prescribing Information Daiichi Sankyo, Inc., December 2019.

### Concomitant Therapy

All concomitant medications, blood products, and radiotherapy administered will be recorded from Day 1 (predose) through the safety reporting period. Any concomitant medication given for a study protocol-related AE should be recorded from the time of informed consent through the safety reporting period.

Any planned surgery (major or minor) not directly related to cancer that occurs on study requires consultation with the sponsor medical monitor. Patients are required to suspend study treatment 3 to 7 days prior to surgery and depending upon the nature of the surgery resume study treatment 3 to 21 days postoperatively. For emergency surgeries, contact medical monitor as soon as feasible to discuss resumption of study treatment postoperatively.

### Required Concomitant Therapy

There are no required concomitant therapies.

### Allowed Concomitant Therapy

Subjects may continue to use any ongoing medications not prohibited by the inclusion/exclusion criteria. However, efforts should be made to maintain stable doses of concomitant medications during the course of study treatment.

During study treatment, subjects may receive supportive care to include bisphosphonates, denosumab, antibiotics, hematologic support, pain management, antacids, and laxatives.

Supportive care medications such as anti-diarrheals and anti-emetics are permitted. Prophylactic use of anti-diarrheals are permitted at the discretion of the investigator. Prophylactic and symptomatic treatment of nausea and vomiting may be used per standard of care.

Thoracentesis or paracentesis may be performed, if needed for comfort.

If surgery or localized radiation become indicated (either for palliation or down-staging of previously nonresectable tumor), these concomitant procedures are permitted for non-target non-CNS lesions only in situations where other disease remains assessable by RECIST 1.1. These interventions should be avoided if clinically feasible until after the second response assessment. The medical monitor should be consulted prior to the intervention occurring.

Corticosteroids
Subjects requiring systemic corticosteroids for control of CNS metastases at a dose >2 mg of dexamethasone (or equivalent) on the first day of study treatment are not eligible to begin study treatment and should not be enrolled until doses <2 mg can be achieved.
After initiation of study treatment, corticosteroids may be initiated for control of CNS symptoms in consultation with the medical monitor.
Premedication with corticosteroids solely for contrast use in CT or MM scans can be used without prior medical monitor approval.
Systemic corticosteroids for control of other comorbidities (e.g., asthma or auto-immune diseases) are permitted.

Blood products and growth factors should be utilized as clinically warranted and following institutional policies and recommendations

Routine prophylaxis with vaccines (without live virus) are permitted during the study

### Prohibited Concomitant Therapy

The following therapies are prohibited during the study (unless otherwise noted):

Investigational drugs and devices

Anti-cancer therapy, including but not limited to chemotherapy and hormonal therapy

Radiation therapy, except for palliative radiotherapy at focal non-CNS sites which are not considered target lesions per RECIST 1.1, which may be given after consultation with the medical monitor, provided that there remain other sites of disease assessable by RECIST 1.1

Vaccination with live vaccines

Strong inhibitors or moderate/strong inducers of CYP2C8 are prohibited as concomitant medications during study treatment—Partial and more complete lists of strong inhibitors and moderate/strong inducers may be found in other reference material. For additional information, including drug elimination half-lives of strong inhibitors and inducers.

Strong inducers of CYP3A4 are prohibited as concomitant medications during study treatment—Partial and more complete lists of strong inhibitors and inducers may be found in other reference material. For additional information including drug elimination half-lives of strong inhibitors and inducers.

Concomitant use of sensitive CYP3A substrates should be avoided 1 week prior to first dose of study treatment and during study treatment. Consider using an alternate medication which is not a sensitive CYP3A substrate. If unavoidable, decrease the CYP3A substrate dosage in accordance with approved product labeling.

Concomitant use of tucatinib with digoxin, a P-gp substrate, increases digoxin concentrations, which may increase the risk for digoxin related adverse reactions. Consider reducing the dosage of digoxin or P-gp substrates with a narrow therapeutic index (such as, but not limited to, dabigatran, fexofenadine, and cyclosporine). Refer to the prescribing information of digoxin or other P-gp substrates for dosage adjustment recommendations due to drug interactions.

### Screening/Baseline Assessments

Screening/Baseline assessments will be conducted to establish study baseline status and determine study eligibility. Only subjects who meet all inclusion and exclusion criteria will be enrolled in this study.

Subjects must have confirmed HER2-positive breast cancer, as determined at a CLIA-certified or ISO-accredited local laboratory. HER2 positivity will be defined by the current ASCO/CAP guidelines. Subject medical history includes a thorough review of significant past medical history, current conditions, any prior treatment and response to prior treatment for the subject's breast cancer, and any concomitant medications.

All measurable and evaluable lesions will be assessed and documented at Screening/Baseline (see Section [658]). A contrast MRI of the brain will be performed to evaluate for the presence of brain metastases (see Section [662]). Subjects with brain metastases at study entry may be eligible for study participation if they meet the inclusion/exclusion criteria and the conditions described in Section [653].

A physical examination including height and weight, vital signs, ECOG performance status, clinical laboratory testing, Contrast CT, PET/CT, or MRI, ECHO/MUGA, ECG, hepatitis B and C screening, biomarker evaluation, and pregnancy testing will be done at Screening/Baseline.

### Treatment for Brain Metastases Prior to Study Entry

Subjects with brain metastases at study entry may be eligible for study participation if they meet the eligibility criteria described in Sections 0 and 0. In order to minimize the risk of symptomatic cerebral edema in subjects with brain metastases in this study, subjects with high-risk metastases, including those requiring immediate local therapy, those with rapidly progressing lesions, those requiring corticosteroids at the start of the study (>2 mg of dexamethasone or equivalent per day) for control of CNS symptoms, and those with larger untreated lesions, are excluded from the trial. However, if these subjects are amenable to immediate CNS-directed therapy with either surgery or radiation, they may undergo local therapy and then be eligible for the trial.

Immediate local therapy to the CNS may delay the screening process beyond the 28-day screening window, in which case the requirement for a repeat contrast MRI after completion of local therapy and prior to starting study treatment is as follows:

For subjects who receive brain radiotherapy during the screening period, the original baseline contrast brain MRI will serve as the baseline for comparison for further response assessments.

For subjects who undergo surgical resection of brain metastases during the screening period, a post-operative contrast brain MRI will be performed and will serve as the baseline for comparison for further response assessments.

For subjects with brain metastases discovered during screening or a history of brain metastases, relevant MRI brain reports and CNS treatment records should be obtained and available for CRF source verification.

### Response/Efficacy Assessments

Radiographic scans and additional imaging assessments (if applicable) will be performed at protocol-specified timepoints, or if disease progression is suspected. Efficacy assessments will be made at each timepoint according to RECIST v1.1 (Eisenhauer 2009; Schwartz 2016) by the investigator.

All known sites of metastatic or locally advanced unresectable disease should be assessed by radiographic imaging at Screening/Baseline to document sites of disease and overall tumor burden. Imaging, preferably by high quality spiral contrast CT scan (with oral and/or IV contrast), should include the chest, abdomen, and pelvis, at a minimum; PET/CT (if high quality CT scan is included) and/or MRI scan may also be done as appropriate. If a CT scan with contrast is contraindicated (i.e., in subjects with contrast allergy or impaired renal clearance), a non-contrast CT scan of the chest may be performed instead, with MM scans of the abdomen and pelvis. At the investigator's discretion, other appropriate imaging (e.g., photography for skin lesions, nuclear bone scan imaging for bone lesions) should be used to assess additional known sites of measurable disease. The same imaging modalities employed in Screening/Baseline should be used for all subsequent response assessments during study treatment and in the follow-up period, unless otherwise clinically indicated. If any other radiographic or assessment exam, including pathology from any on-study biopsies or procedures, is conducted per standard of care, the assessment information will be collected in the CRF.

In the event of equivocal progression, for example a new lesion which is small in size (defined as an equivocal new lesion) and no imminent threat to subject safety, all efforts should be made to continue the subject with study treatment until unequivocal progression is documented. Demonstration of an unequivocal new lesion constitutes disease progression.

Subjects' clinical data must be available for CRF source verification. In addition, images will be collected by an ICR facility for possible future analysis. Copies of tumor images must be made available for review by the sponsor (or its designee) upon request. All imaging will be submitted or uploaded to the ICR facility as soon as reasonably possible (e.g., within approximately 2 weeks) following the date of assessment. Refer to the Study Manual for instructions on collecting and submitting tumor imaging studies to the ICR facility.

### Evaluation of Brain Metastases

Contrast MRI scan of the brain will be performed for all subjects at Screening/Baseline to assess tumor burden in the brain and/or dura and identify subjects with brain metastases at baseline. CT of the brain will not be allowed, and subjects with known contraindications to undergoing contrast MRI imaging will be excluded from the study. Subjects are considered to have brain metastases at baseline with any of the following: any history of brain metastases, any brain metastases at baseline, and brain lesions of equivocal significance at baseline.

Only subjects with documented brain metastases at baseline, as defined above, will continue to have follow-up contrast MRIs of the brain on the same schedule as non-CNS response assessments. Contrast MRIs of the brain may also be performed in subjects without known brain metastases if there is clinical suspicion of new brain lesions. All subjects with a history of brain metastases who discontinue study treatment for reasons other than radiographic disease progression will have an additional contrast MRI of the brain at the EOT visit, unless one has been performed within 30 days of discontinuing study treatment or if progression in the brain has already been documented while on the study.

In subjects with baseline brain lesions, at least 1 brain lesion should be included in the baseline RECIST lesion selection as either a target or non-target lesion. As an exception, however, when unsuspected brain metastases are discovered at screening and immediate CNS-directed therapy is administered, treated lesions should not be selected as target lesions but as non-target lesions for the purpose of disease assessment by RECIST v1.1.

All brain imaging will be collected by an ICR facility for possible future analysis. Copies of brain imaging must be made available for review by the sponsor (or its designee), upon request. Images will be submitted or uploaded to the ICR facility as soon as reasonably possible (e.g., within approximately 2 weeks) following the date of assessment. Refer to the Study Manual for instructions on collecting and submitting brain imaging studies to the ICR facility.

### Pharmacokinetic Assessments

Blood samples will be collected in all subjects at baseline, predose, and at 2 hours (±15 minutes) post-dose of tucatinib per the sample collection schedule provided in Table 8.

PK assessments of trough levels of tucatinib drug levels will be performed on Day 1 of Cycles 2, 3, and 6 prior to administration of tucatinib. On Day 1 of Cycles 2 and 3, PK assessments of peak levels of tucatinib will be performed 2 hours (±15 minutes) after administration of tucatinib. For safety lead-in subjects only, an additional post-dose pharmacokinetic assessment will be performed on Cycle 1 Day 12.

The steady state PK of tucatinib will be assessed through the sparse sampling of the peak and trough levels from Cycle 2 to Cycle 6. PK assessment of trough levels will be performed on all subjects on Day 1 of Cycles 2, 3, and 6 prior to drug administration and peak level assessment will be on Day 1 of Cycles 2 and 3 at 2 hours (±15 minutes) post-dose after drug administration. PK samples should continue to be collected on schedule regardless of dose holds or interruptions. The time of tucatinib administration and PK collection will be recorded by the site.

For safety lead-in subjects only, an additional pharmacokinetic assessment will be performed at an unspecified time on Cycle 1 Day 12. The time of the tucatinib administration on the morning of the visit will be recorded by the subject. Subjects will be called the evening before the visit and reminded to record the time of dose. The exact time of the PK sample will also be recorded by the site.

TABLE 8
Pharmacokinetic sample collection timepoints

| Cycle | Day | Plasma Sample | Serum Sample | Time point |
|---|---|---|---|---|
| | 1 | X | X | 0 h (−2 h) prior to administration of tucatinib |
| 1 | 12 | X | X | For safety lead-in subjects only. Record time of |
| | | | | dose and blood draw. |
| 2 | 1 | X | X | 0 h (−2 h) prior to administration of tucatinib |
| | | X | X | 2 h following administration of tucatinib |
| 3 | 1 | X | X | 0 h (−2 h) prior to administration of tucatinib |
| | | X | X | 2 h following administration of tucatinib |
| 6 | 1 | X | X | 0 h (−2 h) prior to administration of tucatinib |

### Example 5: Treatment with Tucatinib Increases Overall HER2 Levels and Membrane Bound HER2 Levels

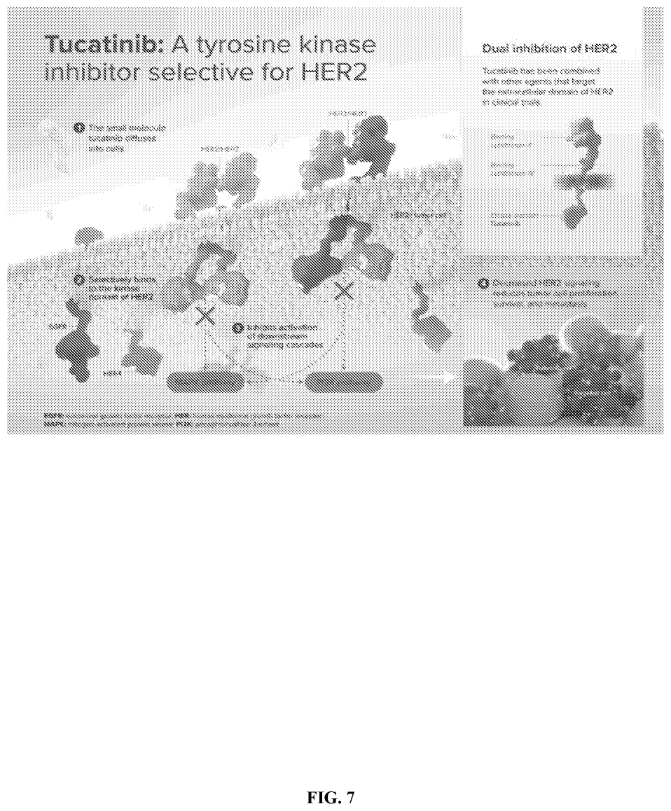

Without being bound by any theory, it is proposed that tucatinib potentiates the activity of T-DM1 by modulating HER2 protein dynamics and facilitating increased cytotoxic maytansinoid drug delivery. FIG. 7 shows a schematic of this proposed mechanism of action for tucatinib. Tucatinib potentiates the activity of T-DM1 by modulating HER2 protein dynamics and facilitating increased cytotoxic maytansinoid drug delivery. At (1) of FIG. 7 tucatinib diffuses into cells and selectively binds to the kinase domain of HER2 (at (2)). At (3), FIG. 7 shows that tucatinib can inhibit activation of downstream signaling cascades (e.g., MAPK pathway and/or P13K pathway). FIG. 7, at (4) shows a schematic that decreased HER2 signaling reduces tumor cell proliferation survival and metastasis.

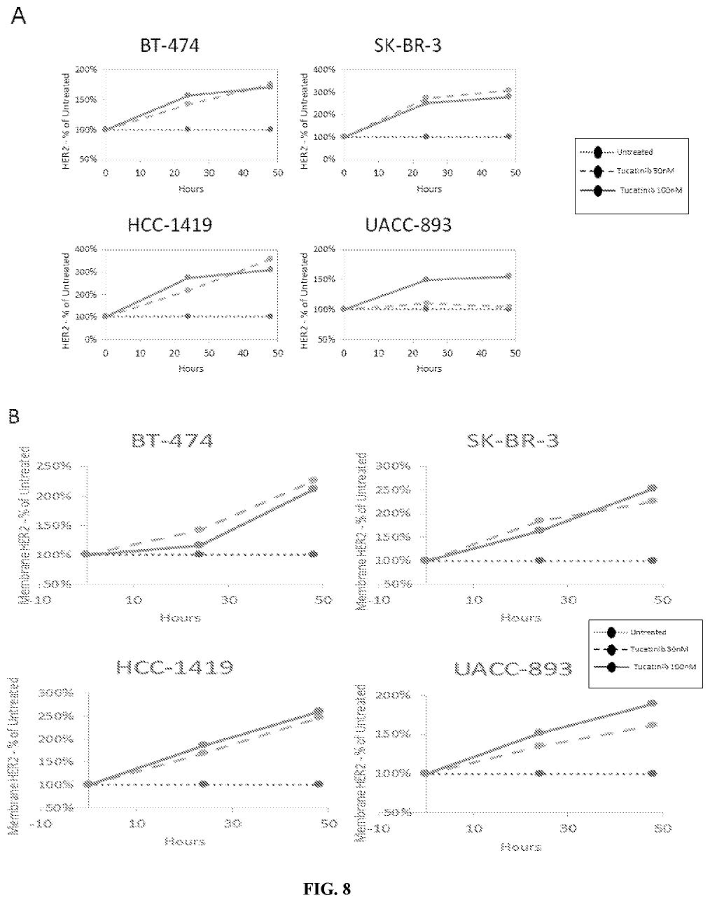

To assess changes to HER2 protein levels upon treatment with tucatinib, HER2-amplified breast cancer cell lines were analyzed by Western blot and quantitative FACS (qFACS). HER2 protein levels were determined for BT-474, SK-BR-3, HCC-1419, and UACC-893 after treatment with tucatinib at either 30 nM or 100 nM doses, for the duration of 24 hours and 48 hours. FIG. 8 shows the changes to total HER2 protein levels and HER2 membrane-bound protein levels upon treatment with tucatinib.

FIG. 8A, shows that in HER2+ breast cancer cell lines, treatment with tucatinib increases overall HER2 levels. HER2 protein levels were determined for BT-474, SK-BR-3, HCC-1419, and UACC-893 after treatment with tucatinib at either 30 nM or 100 nM doses, for the duration of 24 hours and 48 hours. Protein lysates were generated from cells harvested at each timepoint. HER2 total protein levels were determined by western blotting using a WES™ system, and normalized against GAPDH levels as a loading control. In all four cell lines tested, HER2 total protein levels increased after treatment with tucatinib.

FIG. 8B shows that in HER2+ breast cancer cell lines, treatment with tucatinib increases plasma membrane-bound HER2 levels. Cell surface levels of HER2 were determined for BT-474, SK-BR-3, HCC-1419, and UACC-893 after treatment with tucatinib at either 30 nM or 100 nM doses, for the duration of 24 hours and 48 hours. Plasma membrane-associated levels of HER2 were determined by quantitative FACS (qFACS) analysis after exclusion of dead cells. In all four cell lines tested, HER2 levels at the cell surface increased after treatment with tucatinib.

### Example 6: Increased Dwell Time of HER2 at the Cell Surface is Followed by Rapid Internalization and Lysosomal Processing Upon Treatment with Tucatinib

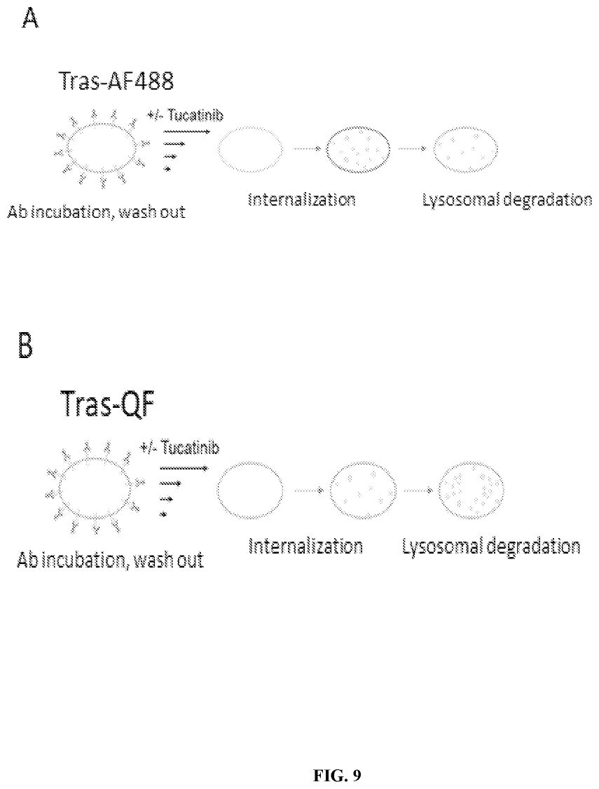

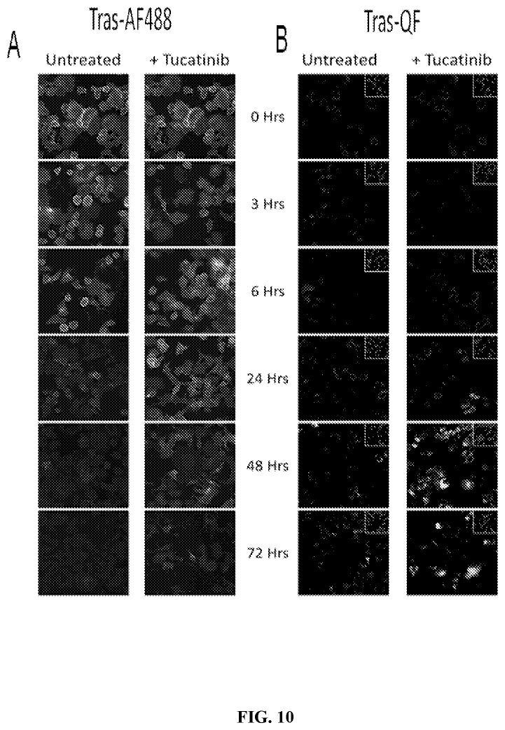

To probe the dynamics of HER2 at the cell surface upon binding to antibody therapeutics, HER2 internalization assays were conducted over the course of 72 hours. FIGS. 9A and 9B shows schematics of internalization assays using Trastuzumab-AF488 and Trastuzumab-QF. FIGS. 10A and 10B show the dynamics of HER2 at the cell surface upon binding to antibody therapeutics.

HER2 Internalization assays using trastuzumab-AF488 are shown in FIG. 9A, and Trastuzumab-QF is shown in FIG. 9B. To probe the dynamics of HER2 at the cell surface upon binding to antibody therapeutics in the presence or absence of tucatinib (100 nM), SK-BR-3 cells were incubated with fluorescently-labeled trastuzumab to mark HER2 at the cell surface as shown in FIGS. 10A and 10B. Excess antibody was washed out. Cells were imaged at time points spanning 72 hours to observe internalization of surface-bound antibody. Concurrent experiments were conducted with trastuzumab labeled with QF in the presence of chloroquine, a quenched fluor which fluoresces upon lysosomal processing and can serve as a proxy for antibody catabolism. Treatment with tucatinib had an initial effect that increased the dwell time of HER2 at the cell surface, potentially mediating increased receptor-binding of antibody therapeutics. At later timepoints, HER2 bound to trastuzumab was internalized and directed towards lysosomes.

### Example 7: Tucatinib Increases Intracellular Payload Concentration when Combined with T-DM1

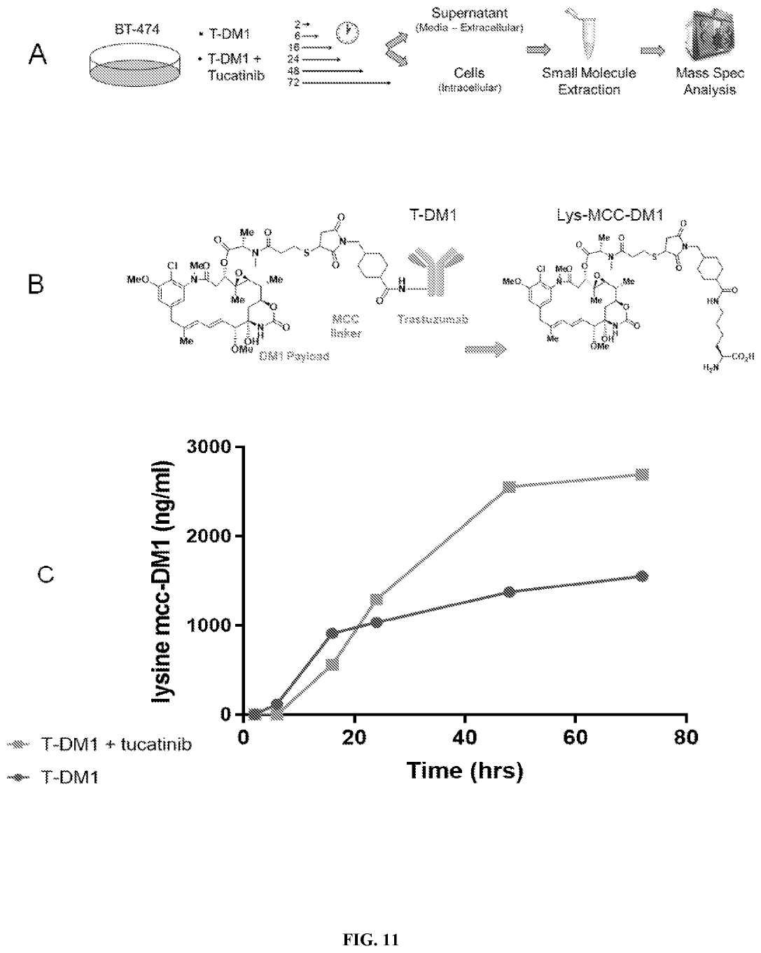

To directly measure the rates of ADC catabolism, cell lysates were analyzed by mass spectrometry for the T-DM1 adduct, Lys-MCC-DM1. FIGS. 11A, 11B, and 11C show a schematic of intracellular drug measurement studies, the structure of the primary T-DM1 catabolite, Lys-MCC-DM1, and the concentration of Lysine-MCC-DM1 over timepoints.

FIG. 11A shows a schematic of intracellular drug measurements studies. The cell lysates (e.g., BT-474) were analyzed by mass spectrometry for the T-DM1 adduct, Lys-MCC-DM1. FIG. 11B shows the structure of the primary T-DM1 catabolite, Lys-MCC-DM1. The cleaved payload was detectible within cells and the majority of T-DM1 is proteolyzed into Lys-MCC-DM1 adduct.

Tucatinib increases the intracellular concentration of DM1 when combined with TDM1 as shown in FIG. 11C. BT-474 breast cancer cells were treated with T-DM1 (3 ug/ml), in either the presence or absence of tucatinib (100 nM). Both supernatant and cells were harvested at timepoints spanning 72 hours for each treatment condition. Samples were analyzed by mass spectrometry to determine the concentration of T-DM1 adducts. Analysis indicated that T-DM1 was predominantly proteolyzed into the Lys-MCC-DM1 adduct, which was predominantly detected within cytoplasm of the cells. Analysis of the Lys-MCC-DM1 adduct within cells demonstrated an increased concentration of DM1 payload in cells treated with T-DM1 in combination with tucatinib, than T-DM1 alone.

### Glossary and Terms

| | |
|---|---|
| 5FU | 5-fluorouracil |
| ADL | activities of daily living |
| AE | adverse event |
| ALT/SGPT | alanine aminotransferase/serum glutamic-pyruvate transaminase |
| ANC | absolute neutrophil count |
| anti-HBc | antibodies to Hepatitis B core |
| anti-HCV | antibodies to Hepatitis C virus |
| API | active pharmaceutical ingredient |
| aPTT | activated partial thromboplastin time |
| AR | adverse reaction |
| AST/SGOT | aspartate aminotransferase/serum glutamic-oxaloacetic transaminase |
| AUC | area under the curve |
| BICR | blinded independent central review |
| BID | twice daily |
| BUN | blood urea nitrogen |
| CBC | complete blood count |
| CBR | clinical benefit rate |
| CHF | congestive heart failure |
| CI | confidence interval |
| Cmax | maximum concentration observed |
| CNS | central nervous system |
| CR | complete response |
| CT | computed tomography |
| CTCAE | Common Toxicity Criteria for Adverse Events |
| ctDNA | circulating tumor DNA |
| DCC | Data Coordinating Center |
| DDI | drug-drug interaction |
| DFS | disease-free survival |
| DMC | Data Monitoring Committee |
| DNA | deoxyribonucleic acid |
| DOR | Duration of Response |
| ECG | electrocardiogram |
| ECHO | echocardiogram |
| ECOG PS | Eastern Cooperative Oncology Group Performance Status |
| eCRF | electronic case report form |
| ED | emergency department |
| EGFR | epidermal growth factor receptor |
| EOI | event of interest |
| EU | European Union |
| FDA | Food and Drug Administration |
| FISH | fluorescence in situ hybridization |
| GCP | Good Clinical Practice |
| GI | gastrointestinal |
| HBsAg | hepatitis B surface antigen |
| HC | Health Canada |
| Hct | hematocrit |
| HER1 | human epidermal growth factor receptor 1 |
| HER2 | human epidermal growth factor receptor 2 |
| HER2+ | human epidermal growth factor receptor 2 positive |
| HIV | human immunodeficiency virus |
| HR | hazard ratio |
| IAR | infusion-associated reaction |
| IB | Investigator's Brochure |
| ICF | Informed Consent Form |
| ICH | International Conference on Harmonisation |
| IHC | immunohistochemistry |
| ILD | interstitial lung disease |
| INR | international normalized ratio |
| IUD | intrauterine device |
| IV | intravenous |
| IRB/IEC | Institutional Review Board/Independent Ethics Committee |
| IRT | Interactive Response Technology |
| ITT | Intent-to-Treat |
| kg | kilogram |
| LDH | lactate dehydrogenase |
| LFT | liver function test |
| LMD | leptomeningeal disease |
| LVEF | left ventricular ejection fraction |
| MedDRA | Medical Dictionary for Regulatory Activities |
| mg | milligram |
| mL | milliliter |
| mm | millimeter |
| MRI | magnetic resonance imaging |
| mRNA | messenger ribonucleic acid |
| MTD | maximum-tolerated dose |
| MUGA | multiple-gated acquisition scan |
| NCI | National Cancer Institute |
| ONT-380 | Investigational small molecule inhibitor of HER2 |
| (tucatinib) | |
| ORR | objective response rate |
| OS | overall survival |
| PD | progressive disease |
| PET | positron emission tomography |
| PFS | progression-free survival |
| P-gp | P-glycoprotein |
| PIC | powder in capsule |
| PK | pharmacokinetics |
| PO | oral administration |
| PPE | palmar-plantar erythrodysaesthesia |
| PR | partial response |
| PT | prothrombin time |
| PVP-VA | polyvinylpyrrolidine-vinyl acetate copolymer |
| QTc | corrected QT |
| RANO-BM | Response Assessment in Neuro-Oncology - Brain Metastases |
| RD | recommended dose |
| RECIST | Response Evaluation Criteria In Solid Tumors |
| RNA | ribonucleic acid |
| RP2D | recommended Phase 2 dose |
| SAE | serious adverse event |
| SAP | statistical analysis plan |
| SD | stable disease |
| SOC | system organ class |
| SRS | stereotactic radiosurgery |
| SUSAR | suspected unexpected serious adverse reaction |
| T-DM1 | ado-trastuzumab emtansine or trastuzumab emtansine |
| TEAE | treatment-emergent adverse event |
| TKI | tyrosine kinase inhibitor |
| Tucatinib | Investigational small molecule inhibitor of HER2 |
| (ONT-380) | |
| UGT1A1 | UDP-glucuronosyltransferase 1A1 |
| ULN | upper limit of normal |
| WBRT | whole brain radiation therapy |

What is claimed is:

1. A method of treating or ameliorating a HER2 positive breast cancer in a subject in need thereof, the method comprising administering to the subject a therapeutically effective amount of a combination therapy comprising tucatinib and an anti-HER2 antibody-drug conjugate.

2. A method of treating or ameliorating cancer in a subject in need thereof, the method comprising:
(a) identifying the subject as having a HER2 positive breast cancer; and
(b) administering to the subject a therapeutically effective amount of a combination therapy comprising tucatinib and an anti-HER2 antibody-drug conjugate.

3. A method for treating or ameliorating a HER2 positive breast cancer in a subject in need thereof, the method comprising administering to the subject an effective amount of a combination therapy comprising tucatinib and an anti-HER2 antibody-drug conjugate, wherein following administration of the combination therapy, the subject exhibits progression-free survival of at least 7.5 months following administration of the combination therapy.

4. The method of claim 3, wherein the subject exhibits progression-free survival of at least eight months following administration of the combination therapy.

5. The method of any one of claim 3 or 4, wherein the subject exhibits progression-free survival of at least nine months following administration of the combination therapy.

6. The method of any one of claims 3-5, wherein the subject exhibits progression-free survival of at least ten months following administration of the combination therapy.

7. The method of any one of claims 1-6, wherein the subject has a brain metastasis.

8. A method for treating or ameliorating a HER2 positive breast cancer in a subject in need thereof, the method comprising administering to the subject an effective amount of a combination therapy comprising tucatinib and an anti-HER2 antibody-drug conjugate, wherein following administration of the combination therapy, the subject exhibits an overall survival of at least eighteen months following administration of the combination therapy.

9. The method of claim 8, wherein the subject exhibits an overall survival of at least nineteen months following administration of the combination therapy.

10. The method of any one of claim 8 or 9, wherein the subject exhibits an overall survival of at least twenty-two months following administration of the combination therapy.

11. The method of any one of claims 8-10, wherein the subject exhibits an overall survival of at least twenty-six months following administration of the combination therapy.

12. The method of any one of claims 8-11, wherein the subject exhibits an overall survival of at least thirty months following administration of the combination therapy.

13. The method of any one of claims 8-12, wherein the subject has a brain metastasis.

14. A method of treating or ameliorating a brain metastasis in a subject having HER2 positive breast cancer, the method comprising administering to the subject an effective amount of a combination therapy comprising tucatinib and an anti-HER2 antibody-drug conjugate.

15. The method of claim 14, wherein the time to additional intervention for treatment of the brain metastasis in the subject has been increased.

16. The method of any one of claim 14 or 15, wherein the need for additional intervention for treatment of the brain metastasis in the subject has been prevented.

17. The method of any one of claim 15 or 16, wherein the additional intervention is selected from the group consisting of radiation, surgery, and a combination thereof.

18. The method of any one of claims 14-17, wherein regression of an existing brain metastasis in the subject has been promoted.

19. The method of any one of claims 14-18, wherein the size of an existing brain metastasis in the subject has been reduced.

20. A method for treating or ameliorating a HER2 positive breast cancer in a subject in need thereof, wherein the subject has a brain metastasis, the method comprising administering to the subject an effective amount of a combination therapy comprising tucatinib and an anti-HER2 antibody-drug conjugate, wherein following administration of the combination therapy, the subject exhibits progression-free survival of at least 6 months following administration of the combination therapy.

21. The method of claim 20, wherein the subject exhibits progression-free survival of at least seven months following administration of the combination therapy.

22. The method of any one of claim 20 or 21, wherein the subject exhibits progression-free survival of at least nine months following administration of the combination therapy.

23. A method for treating or ameliorating a HER2 positive breast cancer in a subject in need thereof, the method comprising administering to the subject an effective amount of a combination therapy comprising tucatinib and an anti-HER2 antibody-drug conjugate, wherein the subject exhibits a greater than 40% reduction in the risk of disease progression or death as compared to a subject administered the anti-HER2 antibody-drug conjugate alone.

24. The method of claim 23, wherein the subject administered the combination therapy comprising tucatinib and the anti-HER2 antibody-drug conjugate exhibits a greater than 45% reduction in the risk of disease progression or death as compared to a subject administered the anti-HER2 antibody-drug conjugate alone.

25. A method for treating or ameliorating a HER2 positive breast cancer in a subject in need thereof, the method comprising administering to the subject an effective amount of a combination therapy comprising tucatinib and an anti-HER2 antibody-drug conjugate, wherein the subject exhibits a greater than 30% reduction in the risk of death as compared to a subject administered the anti-HER2 antibody-drug conjugate alone.

26. A method for treating or ameliorating a HER2 positive breast cancer in a subject in need thereof, wherein the subject has a brain metastasis, the method comprising administering to the subject an effective amount of a combination therapy comprising tucatinib and an anti-HER2 antibody-drug conjugate, wherein the subject exhibits a greater than 50% reduction in the risk of disease progression or death as compared to a subject administered the anti-HER2 antibody-drug conjugate alone.

27. A method for treating or ameliorating a HER2 positive breast cancer in a subject in need thereof, the method comprising administering to the subject an effective amount of a combination therapy comprising tucatinib and an anti-HER2 antibody-drug conjugate, wherein following administration of the combination therapy for nine months, the subject has an estimated progression-free survival rate of greater than 40%.

28. The method of claim 27, wherein the subject has an estimated progression-free survival rate of greater than 45%.

29. A method for treating or ameliorating a HER2 positive breast cancer in a subject in need thereof, the method comprising administering to the subject an effective amount of a combination therapy comprising tucatinib and an anti-HER2 antibody-drug conjugate, wherein following administration of the combination therapy for twelve months, the subject has an estimated progression-free survival rate of greater than 25%.

30. The method of claim 29, wherein the subject has an estimated progression-free survival rate of greater than 30%.

31. A method for treating or ameliorating a HER2 positive breast cancer in a subject in need thereof, the method comprising administering to the subject an effective amount of a combination therapy comprising tucatinib and an anti-HER2 antibody-drug conjugate, wherein following administration of the combination therapy for fifteen months, the subject has an estimated progression-free survival rate of greater than 20%.

32. The method of claim 31, wherein the subject has an estimated progression-free survival rate of greater than 25%.

33. A method for treating or ameliorating a HER2 positive breast cancer in a subject in need thereof, the method comprising administering to the subject an effective amount of a combination therapy comprising tucatinib and an anti-HER2 antibody-drug conjugate, wherein following administration of the combination therapy for twenty-four months, the subject has an estimated overall survival rate of greater than 35%.

34. The method of claim 33, wherein the subject has an estimated overall survival rate of greater than 40%.

35. A method for treating or ameliorating a HER2 positive breast cancer in a subject in need thereof, the method comprising administering to the subject an effective amount of a combination therapy comprising tucatinib and an anti-HER2 antibody-drug conjugate, wherein following administration of the combination therapy for thirty months, the subject has an estimated overall survival rate of greater than 30%.

36. The method of claim 35, wherein the subject has an estimated overall survival rate of greater than 40%.

37. A method for treating or ameliorating a HER2 positive breast cancer in a subject in need thereof, wherein the subject has a brain metastasis, the method comprising administering to the subject an effective amount of a combination therapy comprising tucatinib and an anti-HER2 antibody-drug conjugate, wherein following administration of the combination therapy for nine months, the subject has an estimated progression-free survival rate of greater than 30%.

38. The method of claim 37, wherein the subject has an estimated progression-free survival rate of greater than 40%.

39. A method for treating or ameliorating a HER2 positive breast cancer in a subject in need thereof, wherein the subject has a brain metastasis, the method comprising administering to the subject an effective amount of a combination therapy comprising tucatinib and an anti-HER2 antibody-drug conjugate, wherein following administration of the combination therapy for twelve months, the subject has an estimated progression-free survival rate of greater than 15%.

40. The method of claim 39, wherein the subject has an estimated progression-free survival rate of greater than 20%.

41. A method for treating or ameliorating a HER2 positive breast cancer in a subject in need thereof, the method comprising administering to the subject an effective amount of a combination therapy comprising tucatinib and an anti-HER2 antibody-drug conjugate and an effective amount of an anti-diarrheal agent.

42. The method of claim 41, wherein the combination therapy and the anti-diarrheal agent are administered concurrently.

43. The method of claim 42, wherein the anti-diarrheal agent is administered prior to administration of the combination therapy.

44. The method of claim 41-43, wherein the subject is exhibiting symptoms of diarrhea.

45. The method of claim 41-43, wherein the subject is not exhibiting symptoms of diarrhea.

46. A method of reducing the severity or incidents of diarrhea, or preventing diarrhea in a subject having a HER2 positive breast cancer and being treated with an effective amount of a combination therapy comprising tucatinib and an anti-HER2 antibody-drug conjugate, the method comprising administering an effective amount of an anti-diarrheal agent prophylactically.

47. The method of claim 46, wherein the combination therapy and the anti-diarrheal agent are administered concurrently.

48. The method of claim 46, wherein the anti-diarrheal agent is administered prior to administration of the combination therapy.

49. A method of reducing the likelihood of a subject developing diarrhea, wherein the subject has a HER2 positive breast cancer and is being treated with an effective amount of a combination therapy comprising tucatinib and an anti-HER2 antibody-drug conjugate, the method comprising administering an effective amount of an anti-diarrheal agent prophylactically.

50. The method of claim 49, wherein the combination therapy and the anti-diarrheal agent are administered concurrently.

51. The method of claim 49, wherein the anti-diarrheal agent is administered prior to administration of the combination therapy.

52. The method of any one of claims 1-51, wherein the tucatinib is administered to the subject at a dose of about 150 mg to about 650 mg.

53. The method of claim 52, wherein the tucatinib is administered to the subject at a dose of about 300 mg.

54. The method of claim 52 or 53, wherein the tucatinib is administered once or twice per day.

55. The method of any one of claims 1-54, wherein the tucatinib is administered twice daily.

56. The method of claim 55, wherein the tucatinib is administered to the subject at a dose of about 300 mg twice per day.

57. The method of any one of claims 1-56, wherein the tucatinib is administered to the subject orally.

58. The method of any one of claims 1-57, wherein the anti-HER2 antibody-drug conjugate is administered to the subject at a dose of about 150 mg to about 400 mg.

59. The method of claim 58, wherein the anti-HER2 antibody-drug conjugate is administered to the subject at a dose of about 200 mg.

60. The method of claim 58 or 59, wherein the anti-HER2 antibody-drug conjugate is administered to the subject subcutaneously.

61. The method of any one of claims 1-60, wherein the anti-HER2 antibody-drug conjugate is administered to the subject at a dose of about 1 mg/kg to about 5 mg/kg.

62. The method of claim 61, wherein the anti-HER2 antibody-drug conjugate is administered to the subject at a dose of about 3.6 mg/kg.

63. The method of claim 61, wherein the anti-HER2 antibody-drug conjugate is administered to the subject at a dose of about 4 mg/kg.

64. The method of claim 61, wherein the anti-HER2 antibody-drug conjugate is administered to the subject at an initial dose of about 4 mg/kg followed by subsequent doses of about 3.6 mg/kg.

65. The method of any one of claims 61-64, wherein the anti-HER2 antibody-drug conjugate is administered intravenously.

66. The method of any one of claims 1-65, wherein the anti-HER2 antibody-drug conjugate is administered once about every 1 week, once about every 2 weeks, once about every 3 weeks, or once about every 4 weeks.

67. The method of claim 66, wherein the anti-HER2 antibody-drug conjugate is administered once about every 3 weeks.

68. The method of any one of claims 1-67, wherein the tucatinib and the anti-HER2 antibody-drug conjugate are administered to the subject on a 21 day treatment cycle.

69. The method of claim 68, wherein the tucatinib is administered to the subject twice per day on each day of the 21 day treatment cycle.

70. The method of any one of claims 68-69, wherein the anti-HER2 antibody-drug conjugate is administered to the subject once per 21 day treatment cycle.

71. The method of claim 70, wherein the dose of the anti-HER2 antibody-drug conjugate during the first 21 day treatment cycle is 4 mg/kg and the dose of the anti-HER2 antibody-drug conjugate during the subsequent 21 day treatment cycles is 3.6 mg/kg.

72. The method of any one of claims 1-71, wherein the HER2 positive breast cancer is unresectable or metastatic.

73. The method of any one of claims 1-72, wherein the subject was previously treated with at least one anticancer therapy.

74. The method of claim 73, wherein the least one anticancer therapy is an anti-HER2 antibody or anti-HER2 antibody-drug conjugate.

75. The method of claim 73, wherein the at least one previous anticancer therapy is selected from the group consisting of trastuzumab, trastuzumab and a taxane, pertuzumab, ado-trastuzumab, and combinations thereof.

76. The method of any one of claims 73-75, wherein the subject is refractory to the previous anticancer therapy.

77. The method of claim 71-76, wherein the subject developed a brain metastasis during the previous anticancer therapy.

78. The method of any one of claims 1-77, wherein the subject has not been treated with another therapeutic agent for the breast cancer within the past 12 months.

79. The method of any one of claims 1-78, wherein the subject was previously treated with two or more anti-HER2-based regimens.

80. The method of any one of claims 1-79, wherein the subject has not previously been treated with another therapeutic agent for the breast cancer.

81. The method of any one of claims 1-80, wherein the subject has not previously been treated with lapatinib, neratinib, afatinib, or capecitabine.

82. The method of any one of claims 1-81, wherein the subject has not been previously treated with an anti-HER2 and/or an anti-EGFR tyrosine kinase inhibitor.

83. The method of claim 82, wherein the wherein the anti-HER2/EGFR tyrosine kinase inhibitor is selected from the group consisting of tucatinib, lapatinib, neratinib, or afatinib.

84. The method of any one of claims 1-83, wherein the subject has not been previously treated with an anti-HER2 antibody-drug conjugate.

85. The method of claim 84, wherein the anti-HER2 antibody-drug conjugate is selected from the group consisting of ado-trastuzumab, or trastuzumab deruxtecan.

86. The method of any one of claims 1-85, wherein the subject has not been previously treated with an anthracycline.

87. The method of claim 86, wherein the anthracycline is selected from the group consisting of doxorubicin, epirubicin, mitoxantrone, idarubicin, liposomal doxorubicin, and combinations thereof.

88. A method for treating a HER2 positive breast cancer in a subject that has exhibited an adverse event after starting treatment with a combination therapy comprising tucatinib and anti-HER2 antibody-drug conjugate at an initial dosage level, comprising administering to the subject at least one component of the combination therapy at a reduced dosage level.

89. The method of claim 88, wherein the tucatinib is administered to the subject at an initial dose of about 150 mg to about 650 mg.

90. The method of any one of claim 88 or 89, wherein the tucatinib is administered to the subject at an initial dose of about 300 mg.

91. The method of any one of claims 88-90, wherein the tucatinib is administered to the subject at a reduced dose of about 125 mg to about 275 mg.

92. The method of any one of claims 88-91, wherein the tucatinib is administered to the subject at a reduced dose of about 250 mg.

93. The method of any one of claims 88-91, wherein the tucatinib is administered to the subject at a reduced dose of about 200 mg.

94. The method of any one of claims 88-91, wherein the tucatinib is administered to the subject at a reduced dose of about 150.

95. The method of claim 88-94, wherein the anti-HER2 antibody-drug conjugate is administered to the subject at an initial dose of about 3 mg/kg to about 7 mg/kg.

96. The method of any one of claims 88-95, wherein the anti-HER2 antibody-drug conjugate is trastuzumab deruxtecan.

97. The method of any one of claims 88-95, wherein the anti-HER2 antibody-drug conjugate is ado-trastuzumab emtansine.

98. The method of claim 96, wherein the trastuzumab deruxtecan is administered to the subject at an initial dose of about 5.4 mg/kg.

99. The method of claim 96 or 98, wherein the trastuzumab deruxtecan is administered to the subject at a reduced dose of about 4.4 mg/kg.

100. The method of claim 96 or 98, wherein the trastuzumab deruxtecan is administered to the subject at a reduced dose of about 3.2 mg/kg.

101. The method of claim 97, wherein the ado-trastuzumab emtansine is administered to the subject at an initial dose of about 3.6 mg/kg.

102. The method of claim 97 or 101, wherein the ado-trastuzumab emtansine is administered to the subject at a reduced dose of about 3 mg/kg.

103. The method of claim 97 or 101, wherein the ado-trastuzumab emtansine is administered to the subject at a reduced dose of about 2.4 mg/kg.

104. The method of any one of claims 1-95, wherein the anti-HER2 antibody-drug conjugate is ado-trastuzumab emtansine.

105. The method of any one of claims 1-95, wherein the anti-HER2 antibody-drug conjugate is trastuzumab deruxtecan.

106. The method of any one of claims 1-105, wherein the administration of the tucatinib, or a salt or solvate thereof, increases the overall amount of HER2 in a solid tumor.

107. The method of claim 106, wherein the overall amount of HER2 in the solid tumor is determined by western blot analysis.

108. The method of any one of claims 1-107, wherein the administration of the tucatinib, or a salt or solvate thereof, increases the amount of membrane-bound HER2 in a solid tumor.

109. The method of claim 108, wherein the amount of membrane-bound HER2 in the solid tumor is determined by quantitative fluorescence activated cell sorting (qFACS).

110. The method of any one of claims 1-109, wherein the administration of the tucatinib, or a salt or solvate thereof, increases dwell time of HER2 at the cell surface.

111. The method of any one of claims 1-110, wherein the administration of the tucatinib, or a salt or solvate thereof, increases internalization of membrane-bound HER2.

112. The method of any one of claims 1-111, wherein the administration of the tucatinib, or a salt or solvate thereof, increases lysosomal degradation of HER2.


SEQUENCE LISTING (4 sequences)

SEQ ID NO: 1 - protein, 449 aa, Artificial
  Trastuzumab heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, Artificial
  trastuzumab light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 108 aa, Artificial
  trastuzumab light chain variable domain
DIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQTKVEIKRT

SEQ ID NO: 4 - protein, 120 aa, Artificial
  trastuzumab heavy chain variable domain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS